SEQUENCE LISTING (8 sequences)

SEQ ID NO: 1 - DNA, 19 bp, artificial
  Primer
agcaggtcagcaaagaact

SEQ ID NO: 2 - DNA, 22 bp, artificial
  Primer
cctcatggactgattatggaca

SEQ ID NO: 3 - DNA, 20 bp, artificial
  Primer
gcgaaaagaagtgcagagag

SEQ ID NO: 4 - DNA, 20 bp, artificial
  Primer
cacaaaatgtccaagggaag

SEQ ID NO: 5 - DNA, 21 bp, artificial
  Primer
ctttccaggtcagttagcctt

SEQ ID NO: 6 - DNA, 22 bp, artificial
  Primer
agctctggatgttcttgaagtc

SEQ ID NO: 7 - DNA, 24 bp, artificial
  Primer
agctctgcaaaaagaatagtcatc

SEQ ID NO: 8 - DNA, 22 bp, artificial
  Primer
ataagactgaggggaaccaatg

(19) United States
(12) Patent Application Publication
(10) Pub. No.: US 2016/0311911 A1
(43) Pub. Date: Oct. 27, 2016

(54) METHODS FOR TREATING ALLERGIC AIRWAY INFLAMMATION

(71) Applicants: Sanjiv Sur, Houston, TX (US); Istvan Boldogh, League City, TX (US)

(72) Inventors: Sanjiv Sur, Houston, TX (US); Istvan Boldogh, League City, TX (US)

(21) Appl. No.: 15/136,415

(22) Filed: Apr. 22, 2016

Related U.S. Application Data
(60) Provisional application No. 62/152,368, filed on Apr. 24, 2015.

(51) Int. Cl. C07K 16/28; G01N 33/68; C12N 15/113; A61K 31/17; A61K 9/00

(52) U.S. Cl. CPC C07K 16/2866; A61K 31/17; A61K 9/007; A61K 9/0053; C12N 15/113; G01N 33/6869; C07K 2317/75; C12N 2310/14; C12N 2320/30; G01N 2800/24; G01N 2800/122

(57) ABSTRACT

Provided herein are methods for treating a symptom and/or clinical sign associated with an allergy that includes neutrophil recruitment, such as allergic airway inflammation. In one embodiment, the method includes administering to a subject a composition that includes a CXCR2 inhibitor, a MD2 inhibitor, a MyD88 inhibitor, or a combination thereof. The subject can have an allergy that includes neutrophil recruitment such as allergic airway inflammation, or be at risk of an allergy that includes neutrophil recruitment such as an allergic airway inflammation.

### CROSS-REFERENCE TO RELATED APPLICATIONS

This application claims the benefit of U.S. Provisional Application Ser. No. 62/152,368, filed Apr. 24, 2015, which is incorporated by reference herein.

### GOVERNMENT FUNDING

This invention was made with government support under Grant No. RO1A1062885-06 and RO1ES18468, awarded by the National Institutes of Health. The government has certain rights in the invention.

### SUMMARY OF THE APPLICATION

Provided herein are methods for treating a symptom and/or clinical sign associated with an allergy that includes neutrophil recruitment, such as allergic airway inflammation. In one embodiment, the method includes administering to a subject a composition that includes a CXCR2 inhibitor. The subject can have an allergic airway inflammation or be at risk of an allergic airway inflammation. At least one sign of the allergic airway inflammation or the risk of having the allergic airway inflammation is reduced. The CXCR2 inhibitor can be a CXCR2 antagonist, such as an anti-CXCR2 antibody.

In one embodiment, the allergic airway inflammation includes an allergic asthma. In one embodiment, the allergic airway inflammation includes an allergic rhinitis. In one embodiment, the allergic airway inflammation includes a clinical sign that is allergic conjunctivitis, allergic rhinitis, allergic cutaneous inflammation such as atopic dermatitis, or a combination thereof. In one embodiment, the subject has uncontrolled asthma or controlled asthma. In one embodiment, the subject does not have asthma.

In one embodiment, the allergic airway inflammation includes an inflammatory response to an allergen, wherein the allergen is an aeroallergen. In one embodiment, the aeroallergen is a pollen, an arachnid antigen, a fungal cell, an animal antigen such as animal dander, an insect antigens, or a combination thereof.

Provided herein are methods for using a MD2 inhibitor. In one embodiment, the method includes administering to a subject a composition that includes a MD2 inhibitor. In one embodiment, the method includes administering to a subject a composition that includes an MD2 inhibitor, where the subject has an allergic airway inflammation or is at risk of an allergic airway inflammation. At least one sign of the allergic airway inflammation or the risk of having the allergic airway inflammation is reduced in the subject.

Also provided herein are methods for using a MyD88 inhibitor. In one embodiment, the method includes administering to a subject a composition that includes a MyD88 inhibitor, where the subject has an allergic airway inflammation or is at risk of an allergic airway inflammation. At least one sign of the allergic airway inflammation or the risk of having the allergic airway inflammation is reduced in the subject.

In one embodiment, method described herein also includes determining (i) the level of IL-8 in bronchoalveolar (BAL) fluid of the subject, (ii) the neutrophil percentage in BAL fluid of the subject, or the combination thereof. In one embodiment, the composition can be administered by inhalation, or orally.

### BRIEF DESCRIPTION OF THE FIGURES

FIG. 1. Ragweed pollen extract (RWPE) challenge induces an innate immune response in the airways. (A) Single-challenge model protocol. (B) Bronchoalveolar lavage fluid (BALF) neutrophil number. RWPE challenge in naive wild-type (WT) mice recruited neutrophils into airways 16 and 72 hours after challenge (n=5-7 per group). (C) BALF levels of chemokine (C-X-C motif) ligand (CXCL) 1 and CXCL2 4 hours after challenge. RWPE challenge in naive WT mice increases CXCL1 and CXCL2 in airways 4 hours after challenge. (D) Ex vivo superoxide generation from BALF cells of WT mice. WT mice were killed at 30 minutes and 16 hours after RWPE challenge (n=3-5 per group), and ex vivo superoxide generation from BALF cells was quantified. There was no difference in superoxide generation in any treatment group 30 minutes after challenge. However, at 16 hours after challenge, the RWPE challenge group produced more superoxides. Data are expressed as means±SEM. *P<0.05. **P<0.01. ****P<0.0001. NS, not significant.

FIG. 2. Deletion of Toll-like receptor 4 inhibits RWPE challenge-induced innate immune response. (A) CXCL1 and CXCL2 mRNA expression in lungs. RWPE challenge increased the expression of CXCL1 and CXCL2 mRNA 1 hour after challenge in naive WT mice but not in Tlr4 knockout (KO) mice (n=3 per group). (B) BALF levels of CXCL1 and CXCL2. RWPE challenge increased CXCL1 and CXCL2 levels 4 hours after RWPE challenge in naive WT mice but not in Tlr4 KO mice (n=3-5 per group). (C) BALF neutrophil numbers. RWPE challenge increased the number of neutrophils in BALF at 16 hours after challenge in naive WT mice but not in Tlr4 KO mice (n=5-7 per group). (D) Ex vivo superoxide generation from BALF cells. After PBS or RWPE challenge, ex vivo superoxide generation from BALF was quantified. Ex vivo superoxide generation from BALF cells increased 16 hours after RWPE challenge in naive WT mice but not in Tlr4 KO mice (n=3-5 per group). Data are expressed as means±SEM. *P<0.05. **P<0.01. ****P<0.0001.

FIG. 3. Effect of Toll-like receptor 4 on RWPE-induced allergic airway inflammation. (A) Repeated-challenge model protocol. (B-F) RWPE repeated-challenge model in WT mice and Tlr4 KO mice. (B) BALF eosinophil and total inflammatory cell numbers. Multiple challenges with RWPE induced a greater increase in the number of eosinophils and total inflammatory cells in WT mice compared with Tlr4 KO mice. (C and D) Mucin secretion in airway epithelial cells. Multiple challenges with RWPE induced a greater increase in mucin secretion in WT mice compared with Tlr4 KO mice. (C) Mucin secretion in airway epithelial cells. Original magnification: ×400. (D) Epithelial mucin score. (E) Serum ragweed-specific IgE. Multiple challenge with RWPE induced an increase in serum ragweed-specific IgE in WT mice but not in Tlr4 KO mice. (F) T helper type 2 (Th2) cytokines in BALF. Multiple challenges with RWPE induced an increase in BALF levels of IL-5, IL-13, thymic stromal lymphopoietin (TSLP), and IL-33 in WT mice but not in Tlr4 KO mice. For all groups, 5 to 21 mice per group were used. Data are expressed as means±SEM. *P<0.05. **P<0.01. ***P<0.001.

FIG. 4. Effect of forced neutrophil recruitment in Tlr4 KO mice on RWPE-induced allergic airway inflammation. (A) BALF CXCL1 levels in Tlr4 KO mice. Intranasal challenge with RWPE alone or xanthine with xanthine oxidase (X+XO) alone failed to induce secretion of CXCL1. Administration of a cocktail of RWPE and X+XO induced secretion of CXCL1. (B) BALF neutrophil numbers in Tlr4 KO mice. Intranasal challenge with RWPE alone or X+XO alone failed to increase neutrophil recruitment in BALF 16 hours after challenge. Administration of a cocktail of RWPE and X+XO increased recruitment of neutrophils. (C-E) Effect of repeated RWPE challenge in the presence or absence of X+XO in Tlr4 KO mice. (C) BALF eosinophil and total inflammatory cell numbers in Tlr4 KO mice. RWPE+X+XO challenge increased the number of eosinophils and total inflammatory cells in BALF. (D and E) Mucin secretion in airway epithelial cells of Tlr4 KO mice. RWPE multiple challenges induced a greater increase in mucin secretion in mice challenged with RWPE+X+XO compared with RWPE. (D) Mucin secretion in airway epithelial cells. Original magnification: ×400. (E) Epithelial mucin score. For all groups, five to eight mice per group were used. Data are expressed as means±SEM. **P<0.01. ***P<0.001. ****P<0.0001.

FIG. 5. Effect of chemokine (C-X-C motif) receptor 2 (CXCR2) inhibitor (INH) on RWPE challenge-induced innate and allergic inflammation. (A) Protocol for single-challenge model after intranasal administration of CXCR2 inhibitor. (B) BALF neutrophil numbers in WT mice. Administration of CXCR2 inhibitor before RWPE challenge inhibited neutrophil recruitment 16 hours after challenge (n=5-9 per group). (C) Ex vivo superoxide generation from BALF cells. Intranasal administration of CXCR2 inhibitor before RWPE challenge inhibited ex vivo superoxide generation. *P<0.05 compared with all other groups (n=3-4 per group). (D) Protocol for repeated-challenge model in naive WT mice with or without CXCR2 inhibitor. (E-I) Effect of repeated RWPE challenge in the presence or absence of CXCR2 inhibitor in WT mice (n=5-9 per group). (E) BALF eosinophil and total inflammatory cell numbers. Administration of CXCR2 inhibitor before RWPE challenge inhibited the number of eosinophils and total inflammatory cells. (F and G) Mucin secretion in airway epithelial cells. Administration of CXCR2 inhibitor before RWPE challenge inhibited the increase in mucin secretion. (F) Mucin secretion in airway epithelial cells. Original magnification: ×400. (G) Epithelial mucin score. (H) Serum ragweed-specific IgE. Administration of CXCR2 inhibitor before RWPE challenge inhibited serum ragweed-specific IgE levels. (I) Th2 cytokines in BALF. Administration of CXCR2 inhibitor before RWPE challenge inhibited secretion of IL-5, IL-13, TSLP, and IL-33 in BALF. Data are expressed as means±SEM. *P<0.05. **P<0.01.

FIG. 6. Effect of repeated intranasal administration of neutrophils from donor mice into Tlr4 KO recipient mice after RWPE challenge. (A) Protocol for the repeated-challenge model in Tlr4 KO mice with or without neutrophil replacement 8 hours prior to each instillation of RWPE. (B-F) Effect of repeated RWPE challenge with or without replacement of activated neutrophils in Tlr4 KO mice, assessed on Day 14, 72 hours after the final RWPE challenge on Day 11. (B) BALF eosinophil and total inflammatory cell numbers in Tlr4 KO mice. Intranasal administration of neutrophils after RWPE challenge increased the number of late-phase (72 h) BALF eosinophils and total inflammatory cells. (C and D) Mucin secretion in airway epithelial cells of Tlr4 KO mice. Intranasal administration of neutrophils after RWPE challenge stimulated mucin secretion in Tlr4 KO mice. (C) Mucin secretion in airway epithelial cells. Original magnification: ×400. (D) Epithelial mucin score. (E) Serum ragweed-specific IgE. Intranasal administration of neutrophils after RWPE challenge increased levels of serum ragweed-specific IgE. (F) Th2 cytokines in BALF. Intranasal administration of neutrophils after RWPE challenge increased secretion of IL-5, IL-13, TSLP, and IL-33 in BALF. For all groups, 6 to 18 mice per group were used. Data are expressed as means±SEM. *P<0.05, **P<0.01, ***P<0.001, ****P<0.0001.

FIG. 7. RWPE requires MD2 to induce NF-κB activation, CXCL8 secretion, and neutrophil recruitment. A, FACS of HEK cells cultured with BODIPY-RWPE. B and C, RWPE (FIG. 7B) and Amb a 1 (FIG. 7C) induce CXCL8 secretion from TCM^{Hi} cells. D, RWPE activates NF-κB in TCM^{Hi} cells. E and F, Effect of NF-κB inhibition (FIG. 7 E) and CD14 (FIG. 7F) on RWPE-induced innate inflammation. G, FACS of HEK cells cultured with BODIPY-RWPE. H, RWPE induces CXCL8 secretion from TLR4^{Hi-MD2} cells. I, MD2 siRNA suppresses RWPE-induced CXCL8 secretion from HBECs. J, Tlr4 or Md2 siRNAs suppress RWPE-induced innate inflammation. *P<0.05, **P<0.01, ***P<0.001, and ****P<0.0001. NS, Not significant.

FIG. 8. Diverse pollen allergens require MD2 in addition to TLR4 to induce CXCL8 secretion. A, Specific pollen extracts stimulate CXCL8 secretion from TCM^{Hi} cells. B, Specific pollen extracts stimulate CXCL8 secretion from TLR4^{Hi-MD2} cells. C, MD2 siRNA suppresses pollen extract-induced CXCL8 secretion from HBECs. *P<0.05 and ****P<0.0001. NS, Not significant.

FIG. 9. Requirement of MD2 for CDE-induced recruitment of neutrophils. A, CDE requires CD14, MD2, or both in addition to TLR4 to induce CXCL8 secretion. B, CDE single challenge induces similar neutrophil recruitment in WT and Cd14KO mice. C, CDE induces CXCL8 secretion from TLR4^{Hi-MD2} cells. D, Effect of MD2 siRNA suppression on CDE induced-CXCL8 secretion from HBECs. E, Effect of siRNA knockdown of Md2 on CDE-induced innate inflammation. **P<0.01 and ****P<0.0001. NS, Not significant.

FIG. 10. Effect of suppression of MD2 by means of administration of siRNA on allergic inflammation induced in an RWPE repeated-challenge model. A, Numbers of total inflammatory cells and eosinophils in BALF. B and C, Mucin secretion in airway epithelial cells. FIG. 10B, Original magnification ×400. D, BALF level of IL-5, IL-13, IL-33, and TSLP. E, Serum RWPE-specific IgE. *P<0.05, **P<0.01, and ***P<0.001.

FIG. 11. Experimental protocols used for animal studies. A, Single-challenge model after intranasal administration of NF-κB inhibitor. B, Single-challenge model after single administration of siRNA against Md2 or Tlr4. C, Repeated-challenge model with repeated siRNA administration against MD2 or control siRNA. Each protocol is described in detail in the Methods section.

FIG. 12. FACS analysis of LPS bound to transfected cells. Alexa Fluor 568-labeled LPS binds to TCM^{Hi} but not TLR4^{Hi} TLR4^{Hi-ST}, or TLR4^{Hi-MD2} cells.

FIG. 13. Requirement of Myd88 for cat dander extract-induced recruitment of neutrophils. (A) BALF neutrophil counts in WT and Tlr4 KO mice. (B) BALF neutrophil counts in WT and Tlr2 KO mice. (C) BALF neutrophil counts in WT, TrifKO, and Myd88 KO mice. (D) Cxcl1 and Cxcl2 mRNA expression in lungs. Data are expressed as means±SEM. *=P<0.05, **=P<0.01, ***=P<0.001, ****=P<0.0001.

FIG. 14. Innate immune responses against single cat dander extract challenge. (A) Volcano plot showing significant differential expression of mRNA in WT mice between PBS single challenge and CDE single challenge after 2 hours-post challenge. (B) Pie chart showing protein class and signaling pathway in single challenge model with WT mice after 2 hours-post single CDE challenge.

FIG. 15. Myd88-mediated innate immune responses against single cat dander extract challenge. (A) The fold difference of lung mRNA expression after 2 hours-post single CDE challenge between WT mice and Myd88 KO mice. (B) Pie chart showing the difference of protein class and signaling pathway between WT mice and Myd88 KO mice after 2 hours-post single CDE challenge.

FIG. 16. Effect of disrupting Myd88 on cat dander extract-induced allergic sensitization and inflammation. (A) Number of total inflammatory cells in BALF. (B) Number of eosinophils in BALF, (C,D) mucin secretion in airway epithelial cells. Original magnifications, ×400. (E) Serum total IgE. (F) Serum CDE-specific IgE. (G) BALF level of IL-5, IL-13, IL-33, and TSLP. Data are expressed as means±SEM. *=P<0.05, **=P<0.01, ***=P<0.001, ****=P<0.0001.

FIG. 17. Lung mRNA expression in multiple challenge model with WT mice and Myd88KO mice. (A) Hierarchical clustering analysis showing the change of lung mRNA expression in WT mice in multiple challenge model after 2, 4, and 16 hours-post challenge CDE challenge. (B) Pie chart showing protein class and signaling pathway in multiple challenge model with WT mice after 2 hours-post CDE challenge. (C) The difference of lung mRNA expression in WT mice after 2 hours-post CDE challenge between single CDE challenge model and multiple CDE challenge model. (D) The difference of lung mRNA expression after 2 hours-post CDE challenge in multiple challenge model between WT mice and Myd88 KO mice.

FIG. 18. Effect of disrupting Myd88 on rye grass extract-induced innate immune response and allergic sensitization and inflammation. (A) BALF neutrophil counts in WT and Myd88 KO mice challenged with ragweed, rye grass, and cotton wood. (B) Number of total inflammatory cells in BALF. (C) Number of eosinophils in BALF, (D,E) mucin secretion in airway epithelial cells. Original magnifications, ×400. (F) Serum total IgE. (G) Serum CDE-specific IgE. Data are expressed as means±SEM. *=P<0.05, **=P<0.01, ***=P<0.001,****=P<0.0001.

FIG. 19. (A) Single challenge model protocol. (B) Repeated challenge protocol.

FIG. 20. Differences in cell and cytokine levels in BAL fluids in healthy controls vs. asthma. (A) Percentages of eosinophils and neutrophils in the BAL fluids. (B) Concentrations of IL-1RA, IL-1α, IL-1β, IL-2Rα, IL-5, IL-6, IL-7, IL-8, G-CSF, CXCL1, CCL4, CXCL9, CCL5, and TRAIL in the BAL fluids. Data are expressed as means±SEM. *=P<0.05, **=P<0.01, ***=P<0.001, ****=P<0.0001.

FIG. 21. Correlation of FEV1 to eosinophil, neutrophil, IL-5 and IL-8 levels in asthma. (A) Percentages of neutrophils and concentrations of IL-8 in BAL fluids of subjects with controlled asthma and uncontrolled asthma. (B) Correlations of concentrations of IL-8 with the percentages of neutrophils in the BAL fluid from all subjects with asthma (left panel). Correlation of percentages of neutrophils and concentrations of IL-8 in BAL fluid with percent predicted FEV₁ (middle and right panels, respectively). (C) Correlation of concentrations of IL-5 with the percentages of eosinophil in the BAL fluid from all subjects with asthma (left panel). Correlation of percentages of eosinophils and concentrations of IL-5 in BAL fluid with percent predicted FEV₁ (middle and right panels, respectively). Data are expressed as means±SEM. *=P<0.05, **=P<0.01, ***=P<0.001, ****=P<0.0001.

FIG. 22. Cell and cytokine profile of eosinophil-high (Eos-High) asthma and eosinophil-normal (Eos-Normal) asthma. The upper limit of percent of eosinophils in the BAL fluid of healthy subjects was 0.3%. We separated all subjects with asthma into either eosinophil-high (eosinophils >0.3%) and eosinophil-normal (eosinophils ≦0.3%) groups. Compared to Eos-Normal asthma, Eos-High asthma had higher levels of IL-5 (p<0.05), IL-13 (p<0.05), IL-16 (p<0.05), and PDGF-bb (p<0.05), but same % neutrophils, IL-8, and FEV₁. Data are expressed as means±SEM. *P<0.05, **P<0.01.

FIG. 23. Cell and cytokine profile of neutrophil-high (Neu-High) asthma and neutrophil-normal (Neu-Normal) asthma. The upper limit of percent of neutrophils in the BAL fluid of healthy subjects was 2.4%. We separated all subjects with asthma into neutrophil-high (neutrophils %>2.4%), and neutrophil-normal (neutrophil ≦2.4%) groups. Compared to Neu-Normal asthma, Neu-High asthma had higher IL-8 levels (p<0.01) and lower % predicted FEV₁ (p<0.01), but similar levels of eosinophil %, IL-5, IL-13, IL-16, and PDGF-bb. Data are expressed as means±SEM. *P<0.05, **P<0.01.

### DETAILED DESCRIPTION OF ILLUSTRATIVE EMBODIMENTS

Provided herein are methods for treating a symptom and/or clinical sign associated with allergic airway inflammation. Allergic airway inflammation is a complex chronic inflammatory disease of the airways characterized by airway hyperresponsiveness, infiltration of immune cells into the airway submucosa, mucus hypersecretion, airway remodeling, increased obstruction to airflow, and/or bronchospasm. Two types of allergic airway inflammation are allergic rhinitis and allergic asthma. Allergic rhinitis typically results from exposure to an allergen and includes signs of the condition in the upper respiratory tract (e.g., the regions of the respiratory tract other than the lungs, such as, but not limited to, the nasal terbinates). Allergic asthma, also referred to herein as allergy-triggered asthma, results from exposure to an allergen and includes signs of the condition in the lower respiratory tract (e.g., the lungs). Another type of allergic airway inflammation is severe asthma, also referred to as acute severe asthma. severe asthma is an acute form of asthma that can result from exposure to an allergen. Severe asthma includes signs of the condition in the upper and lower respiratory tract.

Allergic airway inflammation typically includes an allergic reaction to an allergen, e.g., an immune response that includes production of IgE and stimulating histamine release, and late phase recruitment of inflammatory cells such as eosinophils, ILC2 and Th2 cells. Examples of allergens include, but are not limited to, environmental aeroallergens such as plant pollens (e.g., weed, grass, tree); arachnid antigen (e.g. house dust mite); mold/fungal cells (such as a spore); animal antigens (e.g., dander from dog, cat, guinea pig, hamster, gerbil, rat, mouse, etc.); and insect antigens (e.g. flea, cockroach). Examples of grass pollen include, but are not limited to, Bermuda, Rye, and Timothy. Examples of weed pollen include, but are not limited to, ragweed, Pigweed, and Thistle. Examples of tree pollen include, but are not limited to, cedar, mountain cedar, cypress, juniper, cottonwood, and walnut. Allergens may include or be derived from, without limitation, proteins, small molecules, food (typically presenting as a food allergy), drugs (typically presenting as a drug allergy), dust, and proteins and/or carbohydrates of plants, animals, fungi, insects, and mites.

Allergic airway inflammation, such as asthma, may also be triggered by irritants that do not cause an allergic reaction, and such irritants may cause an allergic airway inflammation, or may potentiate an allergic airway inflammation. Examples of irritants include, but are not limited to, lipopolysaccharide (LPS), volatile organic compounds such as formaldehyde and phthalates, cigarette smoke, and air pollution. In one embodiment, an allergic airway inflammation treated using a method described herein is not exacerbated by an irritant.

The methods described herein include administering an effective amount of a composition that includes an inhibitor to a subject. The subject is one having or at risk of having an allergic airway inflammation, such as an allergic asthma or an allergic rhinitis. The inhibitor can be a CXCR2 inhibitor, an MD2 inhibitor, an MyD88 inhibitor, or a combination thereof. CXCR2, also known in the art as IL-8 receptor beta, is a receptor present on neutrophils. MD2 (Myeloid differentiation protein 2) is a glycoprotein that stimulates Toll-like receptor 4 (TLR4) homodimerization-induced canonical inflammatory signaling, and belongs to the MD2-related lipid recognition domain superfamily. MyD88 (Myeloid differentiation primary response gene 88) is a molecule used by the TLR4/MD2 signaling pathway to generate proinflammatory cytokines.

Using a mouse model of allergic airway inflammation, the inventors have found that a CXCR2 inhibitor significantly reduced inflammation resulting from pollen-induced allergic airway inflammation (see Example 1). Additional experimental data indicate inhibitors of MD2 and MyD88 will reduce inflammation resulting from pollen-induced allergic airway inflammation (see Examples 2 and 3).

A CXCR2 inhibitor is a compound that reduces the signaling induced when CXCR2 binds its ligand. In one embodiment, the signaling is CXCR2 mediated beta-arrestin signaling. In one embodiment, the signaling is CXCR2 dependent neutrophil recruitment to the airway epithelial cells. Methods for determining CXCR2 mediated beta-arrestin signaling and CXCR2 dependent neutrophil recruitment are known in the art and are routine (Herrmann et al., WO 2014/170317).

A CXCR2 inhibitor may be a CXCR2 antagonist. A CXCR2 inhibitor reduces or prevents the activity of CXCR2. Methods for measuring the activity of CXCR2 are known in the art. An example of CXCR2 inhibitor that includes a protein is an antibody that binds to CXCR2. Examples of CXCR2 inhibitors are known in the art and include, but are not limited to, Reparixin (Dompe S.P.A.), DF2162 (Dompe S.P.A.), 6 (AstraZeneca), AZ-10397767 (AstraZeneca), SB656933 (GlaxoSmithKline), SB332235 (GlaxoSmithKline), SB468477 (GlaxoSmithKline), SCH527123 (GlaxoSmithKline), AZD5069 (AstraZenca), and SB225002 (Calbiochem) (see Fajas et al., WO2010/092440; Herrmann et al., WO2014/170317; Chapman et al., 2009, Pharmacol. Therapeutics, 121:55-68; and Leaker et al., 2013, Respiratory Res., 14:137).

An MD2 inhibitor is a compound that reduces or prevents the ability of MD2 to stimulate TLR4 after binding LPS presented by CD14. Thus, an MD2 inhibitor can be one that alters binding of LPS by MD2, alters ability of MD2 to interact with CD14, and/or alters stimulation of TLR4 by MD2.

An MD2 inhibitor may be an MD2 antagonist. An MD2 inhibitor reduces or prevents the activity of MD2. Methods for measuring the activity of MD2 are known in the art. An example of an MD2 inhibitor that includes a protein is an antibody that binds to MD2. Examples of MD2 inhibitors are known in the art and include, but are not limited to, curcumin, prenylated flavonoids, rifampin, and eritoran.

A MyD88 inhibitor is a compound that reduces or prevents the ability of MyD88 to act as an adapter protein with the TLR4/MD2 signaling pathway. Thus, a MyD88 inhibitor can be one that alters the ability of MyD88 to bind to TLR4 and/or MD2.

A MyD88 inhibitor may be an MyD88 antagonist. A MyD88 inhibitor reduces or prevents the activity of MyD88. Methods for measuring the activity of MyD88 are known in the art.

A subject can be a patient that has at least one sign or symptom of allergic airway inflammation, is at risk of developing an allergic airway inflammation, e.g., is susceptible to having an allergic airway inflammation and/or has a risk factor for having allergic airway inflammation but has not been exposed to the allergen in a natural environment, or has had an allergic airway inflammation in the past. In one embodiment, the subject has atopy. As used herein atopy includes a genetically mediated predisposition to excessive IgE reactions to an allergen.

As used herein, the term “clinical sign,” or simply “sign,” refers to objective evidence of an allergic airway inflammation present in a subject. As used herein, the term “symptom” refers to subjective evidence of an allergic airway inflammation experienced by the patient and caused by the allergic airway inflammation. Symptoms and/or signs associated with allergic airway inflammation and their evaluation are routine and known in the art. Examples of signs and/or symptoms of allergic airway inflammation include, but are not limited to, airflow obstruction, bronchospasm, wheezing, coughing, chest tightness, and shortness of breath. Other examples include, but are not limited to, allergic conjunctivitis, allergic rhinitis, and allergic cutaneous inflammation like atopic dermatitis.

Treatment of symptoms and/or clinical signs associated with allergic airway inflammation can be prophylactic or, alternatively, therapeutic. Treatment that is prophylactic, for instance, initiated before a subject manifests signs of an allergic airway inflammation, is referred to herein as treatment of a subject that is “at risk” of developing an allergic airway inflammation. An example of a subject that is at risk of developing an allergic airway inflammation is a person having a risk factor and/or is likely to be exposed to an allergen causing the allergic airway inflammation. An example of a risk factor is a known sensitivity to an allergen, has one or more family members with known sensitivity to an allergen, or atopy (e.g., has a genetically mediated predisposition to an excessive IgE reaction to an allergen). For instance, a person with a known sensitivity to pollen allergens is at risk of developing an allergic airway inflammation when exposed to a pollen allergen, and a person with a known sensitivity to cat dander allergens is at risk of developing an allergic airway inflammation when exposed to a cat dander allergen. In another example a subject with atopy is at risk of being sensitized to an allergen to which the subject has not been exposed. Thus, in one embodiment a method herein reduces or prevents sensitization to an allergen. Treatment that is therapeutic is treatment initiated after the subject exhibits one or more symptoms and/or signs associated with allergic airway inflammation. Treatment can be performed before, during, or after the occurrence of an allergic airway inflammation. Treatment initiated after the development of an allergic airway inflammation may result in decreasing the severity of the signs of the allergic airway inflammation, or completely removing the allergic airway inflammation. As used herein, administration of an “effective amount” is an amount effective to prevent the manifestation of symptoms and/or signs of an allergic airway inflammation, decrease the severity of the symptoms and/or signs of an allergic airway inflammation, and/or completely remove the symptoms and/or signs. The subject may be any age, for instance, child or adult, and may be male or female.

Optionally, the method also includes determining whether at least one symptom or sign of the allergic airway inflammation is changed, preferably, reduced.

In one embodiment, the subject has uncontrolled asthma or has controlled asthma. As used herein, a subject with “uncontrolled” asthma refers to a subject that has (i) neutrophil percentage in BAL fluid where the percentage of neutrophils is greater than 2.4% (where the percentage of neutrophils is determined as described in the examples), (ii) an IL-8 concentration in the fluid of a broncheoalveolar lavage (BAL) that is higher by a statistically significant amount compared to adult controls, or both. As used herein, a subject with “controlled” asthma refers to a subject that does not have either (i) or (ii). The method optionally includes determining the neutrophil percentage, determining the IL-8 level in BAL fluid, or a combination thereof.

An inhibitor, such as a CXCR2 inhibitor, an MD2 inhibitor, or an MyD88 inhibitor, is administered to a subject as part of a composition that may further include a pharmaceutically acceptable carrier. In one embodiment, a combination of two of the inhibitors or all three inhibitors can be administered. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. They are generally safe for administering to humans according to established governmental standards, including those promulgated by the United States Food and Drug Administration and the European Medical Agency.

A composition that includes an inhibitor, such as a CXCR2 inhibitor, an MD2 inhibitor, an MyD88 inhibitor, or a combination thereof, and optionally other components may be prepared by methods well known in the art of pharmaceutics. In general, a composition can be formulated into a dosage form that is compatible with its intended route of administration. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. In one embodiment, a route of administration includes mucosal (e.g., nasal, sublingual, vaginal, buccal, or rectal), parenteral (e.g., injection by subcutaneous, intravenous, intramuscular, intradermal), or transdermal administration.

Appropriate dosage forms for enteral administration of the compound of the present invention may include tablets, capsules or liquid solutions. Appropriate dosage forms for parenteral administration may include liquid solutions. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions (where water soluble) or dispersions and sterile powders for the extemporaneous preparation of sterile solutions or dispersions. For parenteral intravenous administration, suitable carriers include physiological saline and the like. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., such as a CXCR2 inhibitor, an MD2 inhibitor, an MyD88 inhibitor, or a combination thereof) in the required amount in an appropriate solvent with one or a combination of ingredients, as required, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, an active compound is delivered in a form that enters the compartments of the lungs. Aqueous or aqueous-organic solutions or suspensions which can be nebulized in combination with propelling agents, such as fluoro chloro hydrocarbons, fluorinated hydrocarbons, dimethyl ether, propane, butane, nitrogen, carbon dioxide, N₂O, or mixtures of powders with microtine drugs, which can be administered using inhalers are suitable. These pharmaceutical compositions may include appropriate additives, such as surface active substances, for instance phospholipids, sorbitane esters, polyoxy sorbitane esters, or oleic acids, alcohols and polyols such as ethanol, glycerol, poly ethylene glycol, glucose, mannitol, sorbitol, in order to improve their relevant pharmaceutical properties.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

A composition including an inhibitor, such as a CXCR2 inhibitor, an MD2 inhibitor, an MyD88 inhibitor, or a combination thereof, may also include other compounds helpful in treating an allergic airway inflammation, such as an allergic airway inflammation, including but not limited to, a beta2-adrenoceptor agonist, an anticholinergic compound, an adrenergic agonist, a corticosteroid, a long-acting beta-adrenoceptor agonist, a leukotriene receptor antagonist (e.g., Singulair), an arachidonate 5-lipoxygenase (5-LOX) enzyme inhibitor, a mast cell stabilizer such as cromolyn sodium, or an H₁ histamine antagonist (e.g., Claritin). The methods described herein may be combined with separate administration of other compounds helpful in treating an allergic airway inflammation.

Toxicity and therapeutic efficacy of an inhibitor, such as a CXCR2 inhibitor, an MD2 inhibitor, an MyD88 inhibitor, or a combination thereof, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred.

The data obtained from cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For a compound used in the methods described herein, the therapeutically effective dose can be estimated initially from cell culture assays. A dose may be formulated in animal models to achieve a concentration range that includes the IC₅₀ (i.e., the concentration of an inhibitor which achieves a half-maximal inhibition of signs of allergic airway inflammation) as determined in cell culture. Such information can be used to more accurately determine useful doses in humans.

The actual dosage amount of an inhibitor administered to a subject can be determined by physical and physiological factors such as body weight, severity of condition, the type of disease being treated, previous or concurrent therapeutic interventions, and the route of administration. The practitioner responsible for administration will, in any event, determine the concentration of inhibitor in a composition and appropriate dose for the subject. The inhibitor may be administered by a practitioner or self-administered by the subject.

In one embodiment, an inhibitor is administered routinely, such as after having a first allergic airway inflammation or after identifying the risk of having an allergic airway inflammation (such as by a standard allergy test or risk factor). Administration of an inhibitor routinely includes a regular course of procedure, including but not limited to once or more than once daily, biweekly, weekly, monthly, and so forth, for example. In one embodiment, an subject may be administered the inhibitor routinely but the dosage may be increased prior to an event or environment where the subject is likely to be or known to be exposed to an allergen that triggers an allergic airway inflammation.

In one embodiment, an inhibitor is administered periodically, such as after having a first allergic airway inflammation or after identifying the risk of having an allergic airway inflammation (such as by a standard allergy test or risk factor). The period may be one or more seasons of the year, one or more periods of time for one or more increased allergens in an environment, such as during pollination of one or more types of plants. In one embodiment, an inhibitor is administered periodically but the dosage may be increased dosage prior to an event or environment where the subject is likely to be or known to be exposed to the allergen.

In one embodiment, an inhibitor is administered prior to an event or environment where the subject is likely to be or known to be exposed to an allergen. For example, the subject may be administered the composition prior to close proximity to or exposure to a particular allergen.

Also provided herein is a kit that includes an inhibitor. The inhibitor can be packaged in aqueous form or lyophilized, in a suitable packaging material and in an amount sufficient for at least one administration. Optionally, other agents can be included for treating an allergic airway inflammation. Instructions for use of the packaged inhibitor can be included.

As used herein, the phrase “packaging material” refers to one or more physical structures used to house the contents of the kit. The packaging material is constructed to provide a sterile, contaminant-free environment. The packaging material has a label which indicates that the inhibitor can be used for treating an allergic airway inflammation.

As used herein an “inhibitor” is a molecule that reduces or prevents the activity of a target molecule, such as a receptor. An “inhibitor” also includes polynucleotides that reduce expression of a target molecule. As used herein, an “antagonist” is an inhibitor molecule that reduces or prevents binding of a ligand to another molecule, such as a receptor. Examples of inhibitors include chemical compounds, including, for instance, an organic compound, an inorganic compound, a metal, a protein (such as an antibody), a non-ribosomal protein, a polyketide, or a peptidomimetic compound.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

Also, in the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### Example 1

### Abstract

Neutrophil recruitment is a hallmark of rapid innate immune responses. Exposure of airways of naive mice to pollens rapidly induces neutrophil recruitment. The innate mechanisms that regulate pollen-induced neutrophil recruitment and the contribution of this neutrophilic response to subsequent induction of allergic sensitization and inflammation need to be elucidated. Here we show that ragweed pollen extract (RWPE) challenge in naive mice induces C-X-C motif ligand (CXCL) chemokine synthesis, which stimulates chemokine (C-X-C motif) receptor 2 (CXCR2)-dependent recruitment of neutrophils into the airways. Deletion of Toll-like receptor 4 (TLR4) abolishes CXCL chemokine secretion and neutrophil recruitment induced by a single RWPE challenge and inhibits induction of allergic sensitization and airway inflammation after repeated exposures to RWPE. Forced induction of CXCL chemokine secretion and neutrophil recruitment in mice lacking TLR4 also reconstitutes the ability of multiple challenges of RWPE to induce allergic airway inflammation. Blocking RWPE induced neutrophil recruitment in wild-type mice by administration of a CXCR2 inhibitor inhibits the ability of repeated exposures to RWPE to stimulate allergic sensitization and airway inflammation. Administration of neutrophils derived from naive donor mice into the airways of Tlr4 knockout recipient mice after each repeated RWPE challenge reconstitutes allergic sensitization and inflammation in these mice. Together these observations indicate that pollen-induced recruitment of neutrophils is TLR4 and CXCR2 dependent and that recruitment of neutrophils is a critical rate-limiting event that stimulates induction of allergic sensitization and airway inflammation. Inhibiting pollen-induced recruitment of neutrophils, such as by administration of CXCR2 antagonists, may be a novel strategy to prevent initiation of pollen-induced allergic airway inflammation.

### Clinical Relevance

The observations in this manuscript indicate that pollen-induced recruitment of neutrophils is Toll-like receptor 4 and chemokine (C-X-C motif) receptor 2 (CXCR2) dependent and that recruitment of neutrophils is a critical rate-limiting event that stimulates induction of allergic sensitization and airway inflammation. Inhibiting pollen-induced recruitment of neutrophils, such as by administration of CXCR2 antagonists, may be a novel strategy to prevent initiation of pollen-induced allergic airway inflammation.

Neutrophil recruitment to an inflamed site is a hallmark of early innate immune responses (1). Allergen challenge in subjects with asthma or seasonal allergic rhinitis has been reported to stimulate IL-8 secretion (2-4) and neutrophil recruitment (5) to the airways. Pollens contain numerous intrinsic factors that can stimulate an innate immune response (6). We and other have reported that intranasal challenge with pollen extract rapidly recruits neutrophils into the airways of naive mice (5, 7-10). However, the innate immune mechanisms that control and regulate pollen-induced recruitment of neutrophils to the airways have not been critically evaluated.

There has been growing interest in defining the additional effects of neutrophils outside of their bacteriocidal effects (11, 12). Of considerable interest is their ability to modulate innate and adaptive immune responses (11, 12). An example of this ability was reported in the recent landmark study that demonstrated that neutrophil recruitment to the skin is essential for induction of subsequent allergic cutaneous inflammation (13). However, the contribution of pollen-induced innate recruitment of neutrophils to subsequent allergic sensitization and airway inflammation has not been critically evaluated. The objectives of the present study were to elucidate the innate mechanisms of the rapid recruitment of neutrophils to the airways within hours of exposure to pollen allergenic extract and to examine the contribution of these recruited neutrophils to the induction of allergic sensitization and airway inflammation.

### Materials and Methods

### Mice

Male wild-type (WT) C57BL/10SNJ mice (8-12 wk old) and Toll-like receptor 4 (Tlr4) knock-out (KO) mice (C57BL/10ScNJ) were purchased from Jackson Laboratory (Bar Harbor, Me.). All mice were maintained in a pathogen-free environment at the University of Texas Medical Branch (Galveston, Tex.). Animal experiments were performed according to the National Institutes of Health Guide for Care and Use of Experimental Animals and were approved by the UTMB Animal Care and Use Committee (approval no. 9708038A).

### Allergenic Extracts

Lyophilized ragweed pollen extract (RWPE) was purchased fromGreer Labs (Lenoir, N.C.). Endotoxin levels in RWPE were quantified using an LAL chromogenic endotoxin quantitation kit (Thermo Scientific, Hudson, N.H.). The levels were exceedingly low (<0.1 pg/μg RWPE protein).

### Protocols Used for Animal Studies

Mice were sedated with low-dose intraperitoneal xylazine-ketamine anesthetic mixture for intranasal sensitization or challenge and killed by lethal intraperitoneal xylazine/ketamine overdose (7, 14).

Single-Challenge Model.

WT mice and Tlr4 KO mice were intranasally challenged with a single dose of 100 μg/60 μl RWPE reconstituted from lyophilized RWPE (Greer Laboratories, Lenoir, N.C.) and killed after 0.5, 1, 4, 16, or 72 hours. In some experiments, to generate superoxide (7, 15), Tlr4 KO mice were intranasally challenged with 0.32 mM xanthine (X) (Sigma-Aldrich, St. Louis, Mo.) with 50 mU xanthine oxidase (XO) (Sigma-Aldrich) in the presence or absence of RWPE. In some experiments, 1 hour before RWPE challenge, WT mice were treated with intranasal administration of 4 mg/kg body weight chemokine (C-X-C motif) receptor 2 (CXCR2) inhibitor SB225002 (Calbiochem, San Diego, Calif.) and challenged with RWPE and killed as described above.

Repeated-Challenge Model.

WT mice or Tlr4 KO mice were sensitized by five intranasal doses of RWPE (100 μg/60 μl) on Days 0, 1, 2, 3, and 4. These mice were challenged with intranasal RWPE or PBS on Day 11 and killed on Day 14 (14). In addition, WT mice were sensitized by five intranasal doses of RWPE on Days 0, 1, 2, 3, and 4 with RWPE with or without the intranasal administration of 4 mg/kg body weight SB225002 (16) 1 hour before each intranasal dose of RWPE. The mice were challenged with intranasal RWPE on Day 11 and killed on Day 14 (14). In some experiments, Tlr4 KO mice were sensitized by five intranasal doses of RWPE on Days 0, 1, 2, 3, and 4 in the presence or absence of X+XO and challenged with intranasal RWPE in the presence or absence of X+XO on Day 11 and killed on Day 14 as described above (14). In some experiments, RWPE and negatively selected neutrophils derived from bone marrow of donor mice were repeatedly intranasally instilled in Tlr4 KO mice. The purity of neutrophils was assessed to be 99% by FACS. Tlr4 KO mice were intranasally administered neutrophils (4×10⁶ neutrophils per mouse) 8 hours after intranasal instillation of RWPE on Days 0, 1, 2, 3, 4, and 11 and killed on Day 14 as described previously to evaluate allergic inflammation.

Isolation and Purification of Neutrophils from Bone Marrow

Bone marrow cells were collected from the femurs and tibias of WT mice (Yasukawa et al., PLoS ONE 2013; 8(5):e64281). The red blood cells were lysed using red blood cell lysing buffer (Sigma), and neutrophils were negatively selected using autoMACS Separator and neutrophil isolation kit mouse (Miltenyi Biotec, Auburn, Calif.).

### Processing of Mouse BALF and Lung Tissue Samples

Bronchoalveolar lavage fluid (BALF) and histological examinations including PAS staining for mucin were performed as described previously (Sur et al., J Immunol 1996; 157(9):4173-4180). In some experiments, leukotriene B4 (LTB4), CXCL1/2, IL-5, IL-13, TSLP, and IL-33 in BALF were assayed using ELISA kits (R&D Systems, Minneapolis, Minn.)

Measurements of Ex Vivo Superoxide Generation from BALF Cells

WT mice and Tlr4 KO mice were challenged with 100 μg of RWPE or PBS and sacrificed at 0.5 and 16 hours. Ex vivo superoxide generation by BALF cells was measured by a mixture of two probes to optimize sensitivity of detection of superoxide (luminol and Diogenes) (Yamazaki et al., Trop Med Health 2011; 39(2):41-45). In additional experiments, WT mice with intranasal administration of the CXCR2 inhibitor SB225002 (4 mg/kg body weight) 1 hour before RWPE challenge, and ex vivo superoxide generation from cells in BALF was quantified.

### Epithelial Mucin Score

Mucin production was assessed by two investigators who were blinded to the treatment groups using a modification of a method reported (Wild et al., 2000, J Immunol 2000; 164(5):2701-2710) on a subjective scale of 0, 1, 2, 3, and 4 corresponding to none, mild, moderate, marked, or severe mucin deposition, respectively. The data were expressed as mean of score recorded by two blinded investigators.

qRT-PCR of Mouse Lung mRNA

RNA was extracted from mouse lung tissue using TRIzol solution (Invitrogen, Carlsbad, Calif.). RNA was reverse-transcribed using SuperScript III First-strand synthesis superMix (Invitrogen). Amplification by real-time PCR was performed on an ABI 7000 Sequence Detection System (Applied Biosystems, Foster City, Calif.) using SYBR Green PCR Master Mix Kit (Applied Biosystems). The primer sequences were as follows:

mouse Hprt, forward, 5′-AGCAGGTCAGCAAAGAACT-3′ (SEQ ID NO:1), reverse, 5′-CCTCATGGACTGATTATGGACA-3′ (SEQ ID NO:2);
mouse CXCL1, forward: 5′-GCGAAAAGAAGTGCAGAGAG-3′ (SEQ ID NO:3), reverse, 5′-CACAAAATGTCCAAGGGAAG-3′ (SEQ ID NO:4);
mouse CXCL2, forward, 5′-CTTTCCAGGTCAGTTAGCCTT-3′ (SEQ ID NO:5), reverse, 5′-AGCTCTGGATGTTCTTGAAGTC-3′ (SEQ ID NO:6).
These primers were obtained from Integrated DNA Technologies (Coralville, Iowa).

### Measurement of RWPE-Specific Serum IgE

RWPE-specific IgE was measured using a previously described procedure (Wild et al., J Immunol 2000; 164(5):2701-2710). Briefly 96-well plates were coated with 100 ug/ml of RWPE protein overnight. After washing and blocking, diluted sera from the mice treated as above were applied. The plates were incubated with biotin-conjugated rat IgE (clone R35-72; BD Biosciences, San Jose, Calif.) for 2 hours at room temperature, washed and incubated with avidin-conjugated alkaline phosphatase. After washing, fluorometric values for each well were recorded after addition of AttoPhos substrate solution (Promega, Madison, Wis.).

### Statistical Analysis

Results of the study are presented as means±SEM. Paired data were analyzed by unpaired t-test. Multiple comparisons were analyzed by ANOVA. The software package GraphPad Prism 6 (GraphPad Software, San Diego, Calif.) was used for all data analyses and preparation of graphs. All statistical analyses considered data significant at p<0.05.

### Results

### A Single RWPE Challenge in Naive Mice Induces Chemokine (C-X-C Motif) Ligand 2 Chemokine Secretion and Neutrophil Recruitment

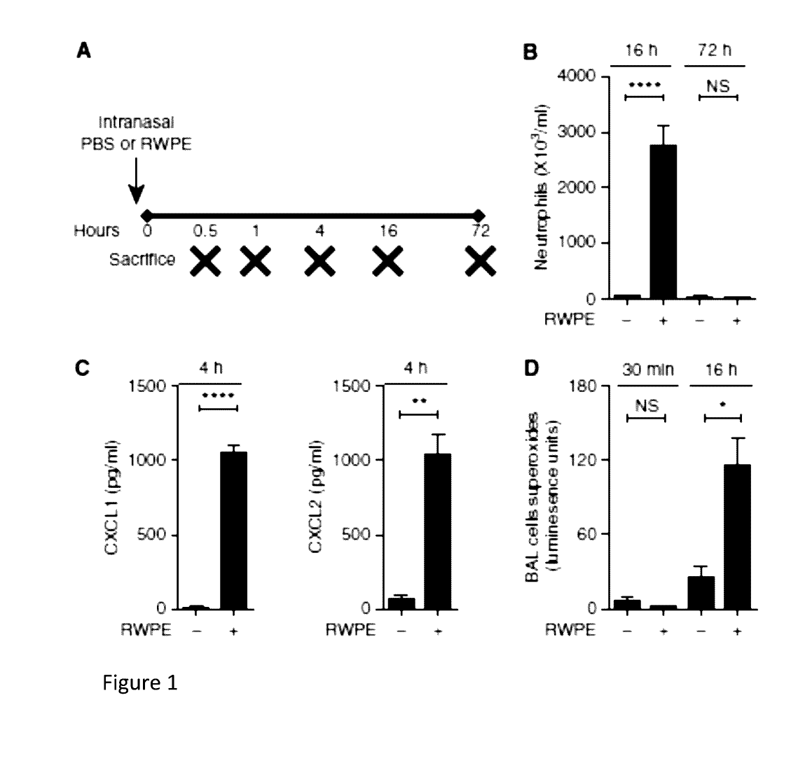

We used RWPE as a model system to define rapid innate events because ragweed pollen allergy is a common cause of human atopic disease (17). All experiments were performed using RWPE with extremely low levels of endotoxin (i.e., <0.1 pg/μg RWPE protein). A single challenge of RWPE (FIG. 1A) in naive C57BL WT mice recruited neutrophils in BALF, peaking (kinetic data not shown) 16 hours after challenge (FIG. 1B). At 72 hours after RWPE challenge, there was no detectable increase in BALF neutrophils (FIG. 1B). At both 16 hours and 72 hours after challenge, there was no detectable increase of eosinophils, lymphocytes, macrophages, or mucin in the airway epithelium (data not shown).

Next we focused our efforts on identifying the mechanism of RWPE-induced neutrophil recruitment into the airways of naive mice. Neutrophil recruitment from blood to a sterile extravascular site is a hallmark of the innate immune response and has been reported to be dependent specifically on leukotriene B4 (LTB4) (1). Secretion of LTB4 from skin tissue has been reported to recruit neutrophils that drive subsequent cutaneous allergic inflammation (13). Based on these earlier reports (1, 13), we initially hypothesized that neutrophil recruitment induced by RWPE challenge was induced by LTB4 secretion. Unexpectedly, RWPE challenge did not induce a measurable increase in BALF LTB4 levels at any time point (data not shown), implying an alternative mechanism of RWPE-induced neutrophil recruitment to the airways. Because neutrophil recruitment is mediated by C-X-C motif ligand (CXCL) chemokines (18), we next investigated whether RWPE induced secretion of CXCL chemokines. A single RWPE challenge (FIG. 1A) in naive WT mice induced secretion of CXCL1 and CXCL2 in BALF in 4 hours (FIG. 1C).

Because deficiency of gp91phox, the major NADPH oxidase in neutrophils, has been reported to decrease reactive oxygen species (ROS) generation in lungs and to induce allergic airway inflammation (19), we next hypothesized that pollen-recruited neutrophils are likely “activated” and generate superoxide. To test this hypothesis, we performed ex vivo analysis of BALF cells for superoxide generation at 30 minutes after challenge when no neutrophils are recruited and at 16 hours after challenge, the peak of neutrophil recruitment, with no other cell types increasing significantly. RWPE challenge in WT mice increased superoxide generation from BALF cells 16 hours after challenge but not at 30 minutes after challenge (FIG. 1D). Together these observations indicate that a single RWPE challenge induces an innate immune response characterized by CXCL chemokine secretion and recruitment of neutrophils and suggest that these recruited neutrophils are likely activated and produce superoxides.

### Toll-Like Receptor 4 Mediates RWPE-Induced CXCL1/2 Secretion and Recruitment of Neutrophils to the Airways

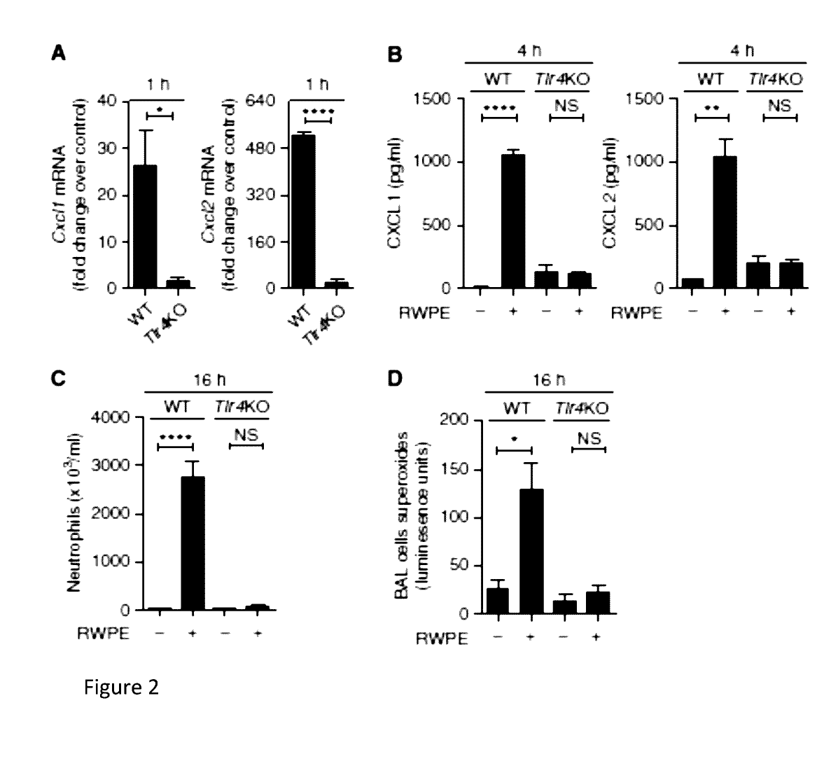

Building on our observations that RWPE induces an innate immune response characterized by CXCL1/2 secretion and recruitment of neutrophils, we attempted to identify the innate mechanism of RWPE-induced CXCL chemokine synthesis. Because stimulation of Toll-like receptor 4 (TLR4) has been shown to induce CXCL chemokines (20), we hypothesized that TLR4-mediated RWPE induces chemokine synthesis. A single RWPE challenge (FIG. 1A) in WT mice increased lung mRNA expression of neutrophil-recruiting CXC chemokines Cxcl1 and Cxcl2 at 1 hour (FIG. 2A), induced secretion of CXCL1 and CXCL2 in BALF at 4 hours (FIG. 2B), and stimulated recruitment of neutrophils at 16 hours (FIG. 2C). Consistent with our hypothesis, deletion of TLR4 reduced CXCL1 and CXCL2 mRNA expression by 95%, secretion of CXCL1 and CXCL2 by 80 to 90%, and recruitment of neutrophils by 97%. Furthermore, compared with superoxide generation from BAL cells at 16 hours in naive WT mice (FIG. 2D), deletion of TLR4 reduced RWPE-induced superoxide generation from BALF cells by 82% (FIG. 2D). These observations indicate that TLR4 regulates RWPE induced innate immune response consisting of CXCL chemokine secretion and recruitment of neutrophils and provide additional evidence that neutrophils are the likely cell source of superoxide generation.

### Blocking RWPE-Induced Recruitment of Activated Neutrophils to the Airways by Deletion of TLR4 Also Blocks Induction of Allergic Airway Inflammation

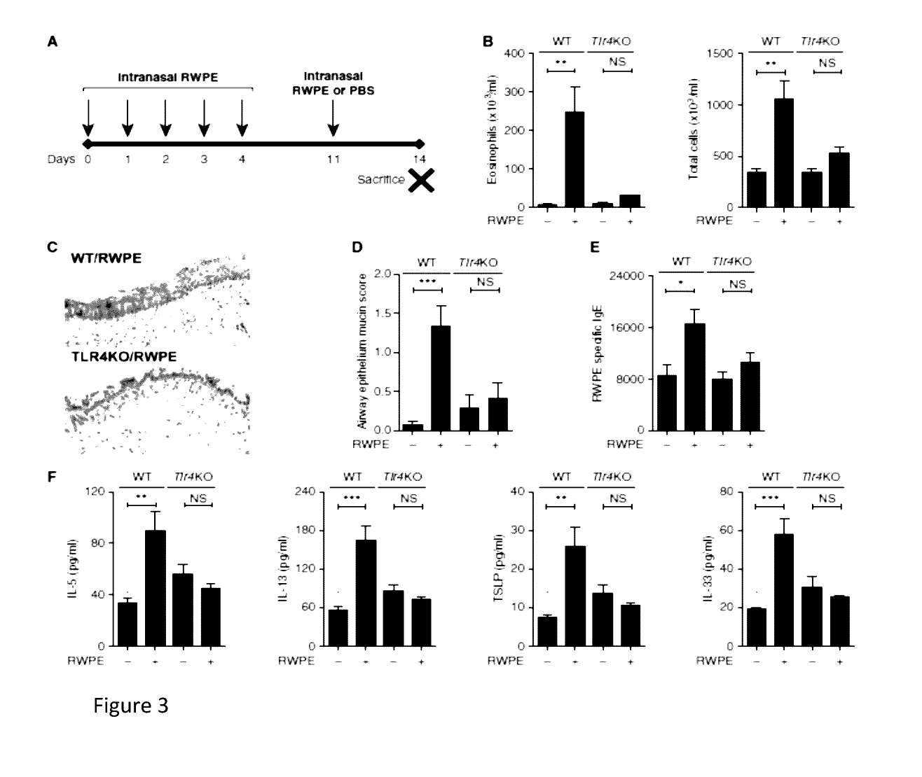

Building on our observation that TLR4 mediates RWPE-induced CXCL chemokine synthesis and recruitment of neutrophils, we used Tlr4 KO mice to test its ability to block allergic airway inflammation. Repeated intrapulmonary exposure of naive mice to RWPE without intraperitoneal injections of alum and allergen mimics chronic exposure of human airways to pollen allergen and facilitates induction of allergic inflammatory response to a later challenge with RWPE (14). To examine the contribution of RWPE-induced innately recruited neutrophils to the development of allergic inflammation, we performed a repeated-challenge model in WT mice and Tlr4 KO mice (FIG. 3A). As expected, in WT mice repeated RWPE challenge increased BALF eosinophils (FIG. 3B), BALF total inflammatory cells (FIG. 3B), airway epithelial mucin secretion (FIGS. 3C and 3D), RWPE-specific IgE in serum (FIG. 3E), and BALF levels of IL-5, IL-13, thymic stromal lymphopoietin (TSLP), and IL-33 (FIG. 3F). By contrast, repeated RWPE challenge in Tlr4 KO mice (FIGS. 3B-3F) failed to increase any of these parameters. These observations indicate that the intensity of allergic inflammation 72 hours after RWPE challenge mirrors the intensity of RWPE-induced innate immune response 1 to 16 hours after challenge, and both are TLR4 regulated. Based on these “mirroring data,” we hypothesized that RWPE-induced innate neutrophil recruitment stimulates allergic sensitization and inflammation.

### Forcing Recruitment of Innate Immune Response in Tlr4 KO Mice Reconstitutes RWPE-Induced Allergic Inflammation

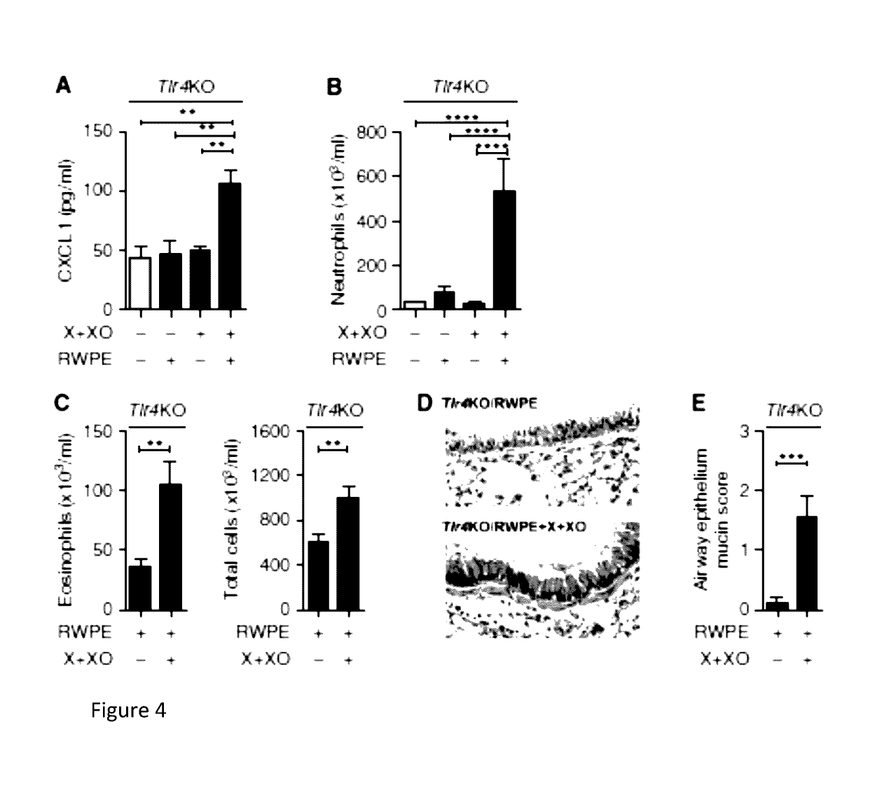

To test this hypothesis, we attempted to force neutrophil recruitment in Tlr4 KO mice to determine if this approach would also reconstitute development of allergic airway inflammation. We initially attempted to force neutrophil recruitment in Tlr4 KO mice by intranasal administration of a superoxide generator, xanthine+xanthine oxidase (X+XO). However, challenge with X+XO failed to stimulate CXCL1 chemokine secretion (FIG. 4A) or to induce neutrophil recruitment 16 hours after challenge (FIG. 4B). Next, we intranasally coadministered a cocktail of RWPE and X+XO. This cocktail successfully stimulated CXCL1 chemokine secretion (FIG. 4A) and restored RWPE-induced neutrophil recruitment in the airways of Tlr4 KO mice (FIG. 4B). We used this ability of RWPE+X+XO to reconstitute neutrophil recruitment in Tlr4 KO mice to test our hypothesis that RWPE challenge-induced repeated recruitment of neutrophils facilitates induction of allergic inflammation. We performed repeated instillation of X+XO or RWPE with or without X+XO into the lungs of Tlr4 KO mice. Repeated challenge with X+XO in isolation failed to induce allergic inflammation (data not shown). By contrast, repeated challenge with a cocktail of RWPE and X+XO also reconstituted allergic inflammation in Tlr4 KO mice (FIGS. 4C-4E). This allergic inflammation was characterized by recruitment of 300% higher eosinophils (FIG. 4C), 160% higher total inflammatory cells (FIG. 4C), and 1,250% higher mucin secretion in airway epithelial cells (FIGS. 4D and 4E). Together these observations give further credibility to our hypothesis that RWPE-induced innate recruitment of neutrophils is a critical rate-limiting event that stimulates induction of pollen-induced allergic airway inflammation.

### CXCR2 Mediates RWPE-Induced Recruitment of ROS-Generating Activated Neutrophils

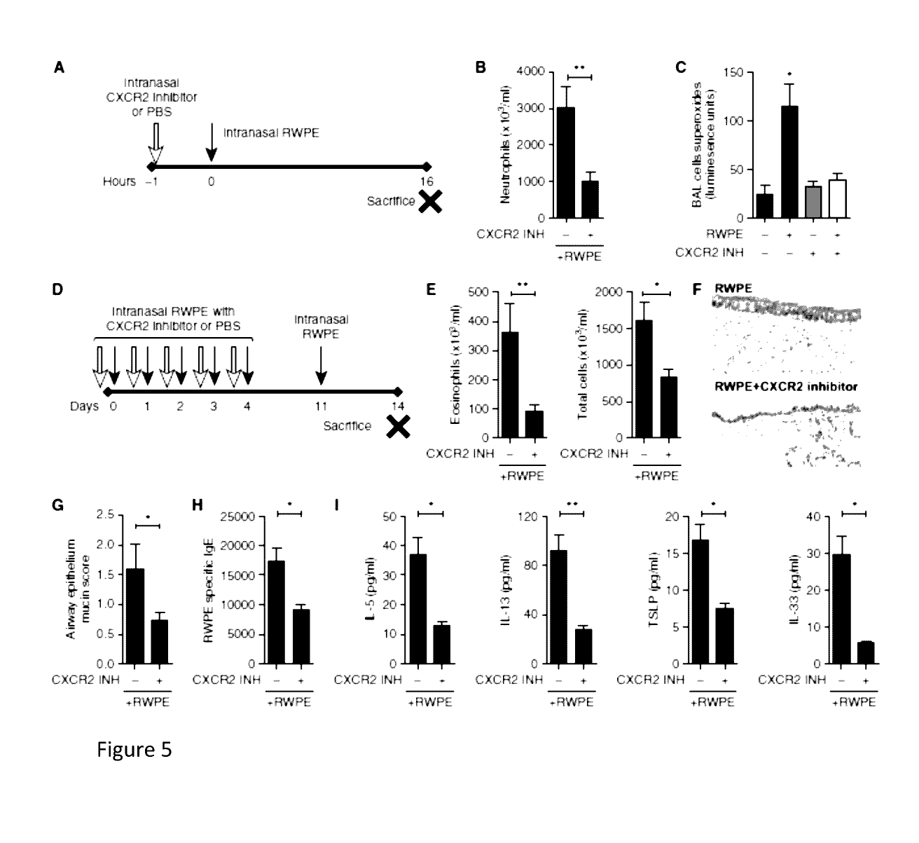

We next focused our efforts on validating our hypothesis that RWPE-induced innate recruitment of neutrophils facilitates induction of allergic airway inflammation. CXCR2, the shared receptor for CXCL1 and CXCL2, has been shown to regulate neutrophilic inflammation (18). SB225002 is a small molecule inhibitor of ligand binding to CXCR2 that has been used by investigators to inhibit CXCR2-driven neutrophilic inflammation (18). To define the role of CXCL1 and CXCL2 in recruitment of neutrophils in our study, we tested the ability of SB225002 to inhibit RWPE-mediated neutrophil recruitment. Administering SB225002 before RWPE challenge in the single-challenge model (FIG. 5A) suppressed neutrophil recruitment into the lungs by 66% (FIG. 5B). Administration of this inhibitor before RWPE challenge did not alter the number of other cell types in BALF (data not shown), indicating specificity of the inhibitor for blocking neutrophil recruitment. To test the contribution of recruited neutrophils to RWPE-induced superoxide generation from BAL cells ex vivo, WT mice were treated with the same dose of SB225002 that inhibited RWPE challenge-induced neutrophil recruitment in FIG. 5B, and ex vivo superoxide generation of BALF cells was quantified 16 hours after RWPE challenge. Administration of CXCR2 inhibitor blocked RWPE-induced recruitment of superoxide-generating cells (FIG. 5C). Together these observations indicate that RWPE challenge induces a CXCR2-dependent recruitment of superoxide generating “activated” neutrophils.

### Repeated CXCR2-Dependent Recruitment of Activated Neutrophils by RWPE Facilitates Induction of Allergic Airway Inflammation

Building on our observation in the present study that a single RWPE challenge induces a CXCR2-dependent recruitment of activated neutrophils to the airways, we hypothesized that repeated recruitment of activated neutrophils after repeated RWPE challenge shifts the immune response to an allergic phenotype. To test this hypothesis, we administered a CXCR2 inhibitor before each of five intranasal RWPE administrations to block repeated recruitment of activated neutrophils. Compared with administering vehicle, administering SB225002 before each of the RWPE instillations in the repeated-challenge model (FIG. 5D) attenuated RWPE-induced allergic inflammation. This attenuation consisted of a 50 to 80% decrease in BALF eosinophils (FIG. 5E); BALF total inflammatory cells (FIG. 5E); accumulation of mucin in epithelial cells (FIGS. 5F and 5G); serum RWPE-specific IgE (FIG. 5H); and BALF levels of IL-5, IL-13, TSLP, and IL-33 in BALF (FIG. 5I). These observations provide further evidence supporting our hypothesis that repeated RWPE-induced recruitment of activated neutrophils contributes to induction of allergic sensitization and airway inflammation in naive mice.

Administration of Neutrophils from Donor Mice into Tlr4 KO Recipient Mice Reconstitutes RWPE-Induced Allergic Sensitization and Airway Inflammation

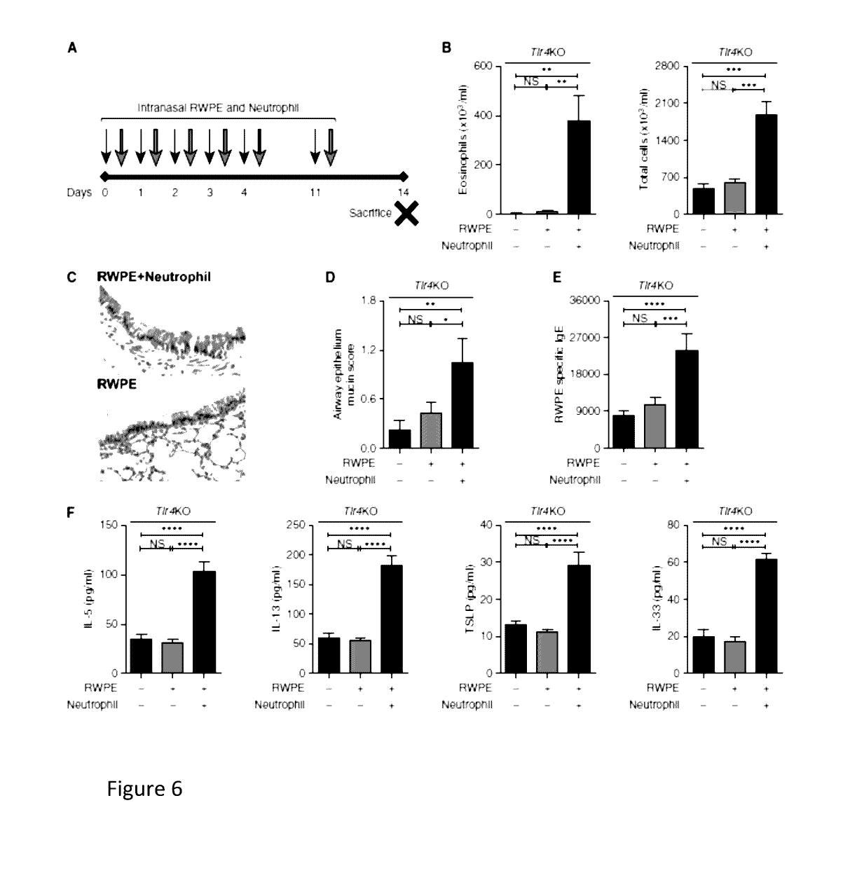

To directly test the role of neutrophils in induction of allergic sensitization and inflammation, we performed a “reconstitution experiment” with repeated intranasal administration of neutrophils derived from naive donor mice to Tlr4 KO recipient mice 8 hours after each RWPE challenge (FIG. 6A). As expected from our data shown in FIG. 3, repeated intranasal administration of RWPE in Tlr4 KO mice without subsequent administration of neutrophils failed to induce allergic sensitization or late-phase allergic airway inflammation (FIGS. 6B-6F). By contrast, repeated intranasal administration of RWPE in Tlr4 KO mice followed by neutrophils reconstituted allergic sensitization and late-phase allergic airway inflammation. This was characterized by an increase in BALF eosinophils (FIG. 6B); BALF total inflammatory cells (FIG. 6B); mucin secretion in airway epithelial cells (FIGS. 6C and 6D); serum levels of RWPE-specific IgE (FIG. 6E); and BALF levels of IL-5, IL-13, TSLP, and IL-33 (FIG. 6F). Together these observations indicate that RWPE-induced innate recruitment of neutrophils is a critical rate-limiting step that is required for induction of allergic sensitization and stimulation of allergic airway inflammation.

### Discussion

Our data indicate that pollen-initiated repeated recruitment of activated neutrophils plays a critical role in pollen-induced induction of allergic sensitization and airway inflammation in a naive host. A recent study demonstrated that LTB4 mediates OVA-induced neutrophil recruitment to the skin, and these recruited neutrophils facilitate induction of subsequent allergic skin inflammation (13). Another study reported that LTB4 specifically mediates recruitment of neutrophils after infection with Pseudomonas aeruginosa to lymph nodes (1). However, in the present study, intranasal challenge of mice with RWPE failed to increase LTB4 levels in the airways. Instead, recruitment of neutrophils by RWPE was regulated by the TLR4-CXCR2 pathway. The difference in mechanism of neutrophil recruitment between the earlier studies and the present study could be due to differences in which organ system was exposed to allergen (skin or lymph nodes in earlier studies [1, 13] versus lung in the present study) or to the type of allergen (OVA or P. aeruginosa in earlier studies [1, 13] versus RWPE in the present study).

Our study demonstrates an interesting novel finding: exposure to pollens induces an innate recruitment of neutrophils to the airways, and this recruitment facilitates allergic sensitization. In the present study, one possible mechanism by which innate recruitment of neutrophils by pollens shifts the immune response to an allergic phenotype upon repeated challenge is induction of a state of sustained oxidative stress in the airways by repeated recruitment of activated ROS-generating neutrophils. This is suggested by reports that mice deficient in gp91phox, the dominant superoxide-generating enzyme in neutrophils, have decreased ROS generation and mount an attenuated allergic inflammatory response to allergen challenge (19). Additionally, chronic oxidative stress can worsen allergic asthma (21-24), alter dendritic cell function, and modify the Th1/Th2 balance (25, 26). Some publications indicate that neutrophils have numerous additional effects that could modulate allergic inflammation, as evidenced by their ability to promote extracellular infection clearance in a regulation of innate and adaptive immune responses (11, 12). Thus, neutrophils may have contributed to allergic sensitization and inflammation in our study by increasing microvascular permeability (27), inducing proinflammatory cytokines (28), matrix metalloproteinase 9 (29), and MUC5AC (30). However, the precise molecular pathways by which neutrophils initiated allergic sensitization in the present study have to be elucidated in future studies.

The role of TLRs in pollen-induced allergic inflammation has been evaluated recently (31, 32). In mice sensitized to RWPE, conjunctival challenge with ragweed pollen extract has been reported to stimulate TLR4-dependent allergic inflammation in murine and human corneal epithelia (32). Likewise, another group reported that the adaptive immune response to birch pollen extract was inhibited in mice lacking TLR4 (31). Our data indicate that the TLR4-CXCR2 pathway may have contributed to allergic inflammation in those studies by controlling pollen-induced recruitment of activated neutrophils. Activation of the TLR4-CXCR2 pathway upon exposure to pollens in our study may explain pollen-induced induction of IL-8 (2-4), recruitment of neutrophils (5), and oxidative stress markers (33-36) reported in earlier studies. Additional studies are needed to determine whether the dominance of neutrophils in the airways in severe asthma (37) and sudden-onset fatal asthma (38) are a consequence of chronic activation of the TLR4-CXCR pathways by pollen allergens. Prior epidemiologic studies have suggested a role of TLR4 in human allergic diseases. Several TLR4 SNPs have been identified that facilitate allergic sensitization and prevalence of asthma (39, 40). Future studies would have to determine whether there are differences in stimulation of pollen-induced TLR4-CXCR2 pathway in individuals with specific TLR4 SNPs, thereby modulating recruitment of activated neutrophils and induction of allergic sensitization and airway inflammation.

CXCR2 has been shown to play a significant role in infection by regulating neutrophil trafficking (41-44). Thus, the ability of CXCR2 inhibitors to inhibit RWPE-mediated allergic inflammation in our study is somewhat surprising because it is not conventionally considered relevant to pollen-induced allergic airway inflammation. However, evidence from murine and human studies suggests an importance of CXCR2 signaling in the induction of asthma and allergic inflammation. Thus, Cxcr2 KO mice sensitized with fungus demonstrate reduced secretion of Th2 cytokines in the airway compared with WT mice (45). Blockade of CXCR2 signaling by neutralizing antibody inhibits OVA-induced airway remodeling in a murine mouse model (46). Treatment of mice with anti-CXCR2 mAb inhibits IL-33-induced late-phase airway obstruction, airway hyperresponsiveness, eosinophilic inflammation, and goblet cell hyperplasia (47). Additionally, treatment of human subjects with severe asthma with CXCR2 inhibitor SCH 527123 reduced asthma exacerbations (48). Because none of these studies focused on the importance of pollen-induced recruitment of neutrophils in the induction of allergic sensitization and airway inflammation (46-48), our study provides crucial mechanistic data that might explain some of the observations reported in the earlier studies. Because TSLP and IL-33 can induce Th2 polarization (49, 50) and because our study indicates that repeated administration of RWPE and neutrophils in Tlr4 KO mice increases BALF levels of these cytokines, it is likely that either neutrophils, a factor derived from neutrophils, or oxidative damage to airway structural cells by neutrophils induces secretion of these cytokines, which in turn stimulate allergic sensitization and inflammation.

Our observations indicate that pollen-induced repeated recruitment of neutrophils is a critical rate-limiting event that stimulates allergic sensitization and induction of pollen-induced allergic airway inflammation. Furthermore, our study provides a mechanistic link between TLR4 and CXCR2 in pollen-induced recruitment of neutrophils. We suggest that inhibiting recruitment of activated neutrophils by administration of CXCR2 or other antagonists may be a unique strategy for preventing allergic sensitization and pollen-induced allergic disorders.

### REFERENCES FOR EXAMPLE 1

1. Lammermann et al., Nature 2013; 498: 371-375.
2. Virchow et al., Am J Respir Crit Care Med 1995; 151:960-968.
3. Teran et al., Am J Respir Crit Care Med 1996; 154:469-476.
4. KleinJan et al., J Allergy Clin Immunol 1999; 103:441-450.
5. Lommatzsch et al., J Allergy Clin Immunol 2006; 118:91-97.
6. Hosoki et al., Curr Opin Allergy Clin Immunol 2015; 15:79-88.
7. Boldogh et al., J Clin Invest 2005; 115:2169-2179.
8. Hoskins et al., Am J Respir Crit Care Med 2013; 187:34-41.
9. Metzger et al., Chest 1986; 89:477-483.
10. Imai et al., Cell 2008; 133:235-249.
11. Mantovani et al., Nat Rev Immunol 2011; 11:519-531.
12. Mocsai, J Exp Med 2013; 210:1283-1299.
13. Oyoshi et al., Immunity 2012; 37:747-758.
14. Wild et al., J Immunol 2000; 164:2701-2710.
15. Dharajiya et al., J Allergy Clin Immunol 2007; 119:646-653.
16. Ijichi et al., J Clin Invest 2011; 121:4106-4117.
17. White et al., Ann Allergy Asthma Immunol 2003; 91:425-435.
18. Braber et al., Eur J Pharmacol 2011; 668:443-449.
19. Sevin et al., Am J Respir Cell Mol Biol 2013; 49:396-402.
20. De Filippo et al., Blood 2013; 121:4930-4937.
21. McDonnell et al., Environ Res 1999; 80:110-121.
22. Kim et al., Ann Allergy Asthma Immunol 2011; 107:214-219.
23. McConnell et al., Lancet 2002; 359:386-391.
24. Yamazaki et al., J Expo Sci Environ Epidemiol 2014; 24:372-379.
25. Peterson et al., Proc Natl Acad Sci USA 1998; 95:3071-3076.
26. Csillag et al., J Immunol 2010; 184:2377-2385.
27. Chignard et al., Am J Physiol Lung Cell Mol Physiol 2000; 279:L1083-L1090.
28. Sibille et al., Eur Respir J 1993; 6:1529-1543.
29. Park et al., J Immunol 2006; 176:2538-2545.
30. Voynow et al., Am J Physiol 1999; 276:L835-L843.
31. Shalaby et al., J Immunol 2013; 191:922-933.
32. Li et al., J Allergy Clin Immunol 2011; 128:1318-1325.
33. Calhoun et al., Am Rev Respir Dis 1992; 145:317-325.
34. Gratziou et al., Int J Immunopathol Pharmacol 2008; 21:949-957.
35. Comhair et al., Lancet 2000; 355:624.
36. Dworski et al., Clin Exp Allergy 2001; 31:387-390.
37. Jatakanon et al., Am J Respir Crit Care Med 1999; 160:1532-1539.
38. Sur et al., Am Rev Respir Dis 1993; 148:713-719.
39. Werner et al., J Allergy Clin Immunol 2003; 112:323-330.
40. Kerkhof et al., Thorax 2010; 65:690-697.
41. Wareing et al., Viral Immunol 2007; 20:369-378.
42. Hall et al., J Immunol 2001; 166:4035-4041.
43. Tsai et al., Infect Immun 2000; 68:4289-4296.
44. Johnston et al., Am J Physiol Lung Cell Mol Physiol 2005; 288:L61-L67.
45. Schuh et al., J Immunol 2002; 168:1447-1456.
46. Jones et al., Stem Cells 2009; 27:3074-3081.
47. Mizutani et al., J Immunol 2014; 192:1372-1384.
48. Nair et al., Clin Exp Allergy 2012; 42:1097-1103.
49. Froidure et al., Allergy 2014; 69:1068-1076.
50. Schmitz et al., Immunity 2005; 23:479-490.

### Example 2

Background: The National Health and Nutrition Examination Survey identified several pollens and cat dander as among the most common allergens that induce allergic sensitization and allergic diseases. We recently reported that ragweed pollen extract (RWPE) requires Toll-like receptor 4 (TLR4) to stimulate CXCL-mediated innate neutrophilic inflammation, which in turn facilitates allergic sensitization and airway inflammation. Myeloid differentiation protein 2 (MD2) is a TLR4 coreceptor, but its role in pollen- and cat dander-induced innate and allergic inflammation has not been critically evaluated.

Objective: We sought to elucidate the role of MD2 in inducing pollen- and cat dander-induced innate and allergic airway inflammation.

Methods: TCM^{Null} (TLR4^{Null}, CD14^{Null}, MD2^{Null}), TLR4^{Hi}, and TCM^{Hi} cells and human bronchial epithelial cells with small interfering RNA-induced downregulation of MD2 were stimulated with RWPE, other pollen allergic extracts, or cat dander extract (CDE), and activation of nuclear factor κB (NF-κB), secretion of the NF-κB-dependent CXCL8, or both were quantified. Wild-type mice or mice with small interfering RNA knockdown of lung MD2 were challenged intranasally with RWPE or CDE, and innate and allergic inflammation was quantified.

Results: RWPE stimulated MD2-dependent NF-κB activation and CXCL secretion. Likewise, Bermuda, rye, timothy, pigweed, Russian thistle, cottonwood, walnut, and CDE stimulated MD2-dependent CXCL secretion. RWPE and CDE challenge induced MD2-dependent and CD14-independent innate neutrophil recruitment. RWPE induced MD2-dependent allergic sensitization and airway inflammation.

Conclusions: MD2 plays an important role in induction of allergic sensitization to cat dander and common pollens relevant to human allergic diseases.

Pollens and cat dander are major causes of allergic airway disorders, such as rhinitis and asthma (1-3). The National Health and Nutrition Examination Survey identified several pollens and cat dander as among the most common allergens that induce allergic sensitization and allergic diseases (2). The role of adaptive immune responses in induction of allergic diseases by allergens has been extensively studied. However, relatively little is known about innate immune receptors that contribute to allergic sensitization. Recent studies have identified a role of innate responses mediated by Toll-like receptor 4 (TLR4) in pollen-induced allergic inflammation (4-6). One study reported that short ragweed pollen induces allergic conjunctivitis by stimulating TLR4-dependent thymic stromal lymphopoietin (TSLP) secretion in sensitized mice (4). Another study reported that adaptive allergic immune responses to birch pollen extract were reduced in Tlr4 knockout (KO) mice, thus implying a role of TLR4 in induction of allergic immune responses (5). Recently, we reported that ragweed pollen extract (RWPE) challenge induces CXCR2- and TLR4-dependent innate recruitment of activated neutrophils to the lungs (6). We reported that deletion of TLR4 abrogated RWPE-induced allergic sensitization and allergic inflammation (6). We further demonstrated that passive transfer of neutrophils to Tlr4KO recipient mice reconstitutes allergic sensitization and allergic airway inflammation in Tlr4KO mice (6). Myeloid differentiation protein 2 (MD2) is a 160-amino-acid Glycoprotein (7). MD2 directly binds LPS presented by CD14 (8,9) and stimulates TLR4 homodimerization-induced canonical inflammatory signaling (10). MD2 belongs to the MD2-related lipid recognition domain superfamily, which also includes the mite allergens Der p 2 and Der f 2 in addition to MD1, GM2 activator protein, Niemann-Pick C2 protein (Npc2), and phosphatidylinositol phosphatidylglycerol transfer protein. The structural and functional mimicry of MD2 by Der p 2 stimulates TLR4 (11). However, the role of MD2 in pollen-induced innate and allergic airway inflammatory responses has not been reported. We hypothesized that because RWPE requires TLR4 to induce innate inflammation-mediated allergic sensitization (6), it might also require MD2 to mediate these effects. We further hypothesized that this pathway is shared by other common allergens relevant to human allergic diseases (2, 3).

Abbreviations used: BALF, Bronchoalveolar lavage fluid; CDE, Cat dander extract; FACS, Fluorescence-activated cell sorting; HBEC, Human bronchial epithelial cell; KO, Knockout; MD2, Myeloid differentiation protein 2; NF-κB, Nuclear factor κB; RWPE, Ragweed pollen extract; siRNA, Small interfering RNA; TLR4, Toll-like receptor 4; and WT, Wild-type.

### Methods

### Mice

Eight- to 12-week-old male wild-type (WT) C57BL/10SNJ mice, Tlr4 knockout (KO) mice (C57BL/10ScNJ), WT mice (C57BL/6J), and Cd14KO mice (B6.1295-Cd14tm1Frm/J) were purchased from Jackson Laboratory (Bar Harbor, Me.) for these studies. The mice were maintained in a pathogen-free environment at the University of Texas Medical Branch (Galveston, Tex.). Animal experiments were performed according to the National Institutes of Health guidelines and were approved by the University of Texas Medical Branch Animal Care and Use Committee.

### Allergenic Extracts

We have previously reported that lyophilized RWPE containing a very low amount of endotoxin (<0.1 pg of LPS/1 μg of allergen protein; Greer Laboratories, Lenoir, N.C.) induces TLR4-dependent innate neutrophilic inflammation and allergic sensitization (6). For the present study, we purchased lyophilized RWPE, Bermuda grass, timothy grass, rye grass, firebush, pigweed, Russian thistle, black walnut, eastern cottonwood, mountain cedar, and cat dander extract (CDE) from Greer Laboratories. Like our previous study (6) all tested allergenic extracts had very low (<0.1 pg of LPS/1 μg) levels of allergen protein, as determined by using the LAL chromogenic endotoxin quantitation kit (Thermo Scientific, Hudson, N.H.).

### Protocols Used for Animal Studies

Mice were sedated with low-dose intraperitoneal xylazine-ketamine anesthetic mixture for intranasal sensitization or challenge and euthanized by a lethal anesthetic mixture overdose (12).

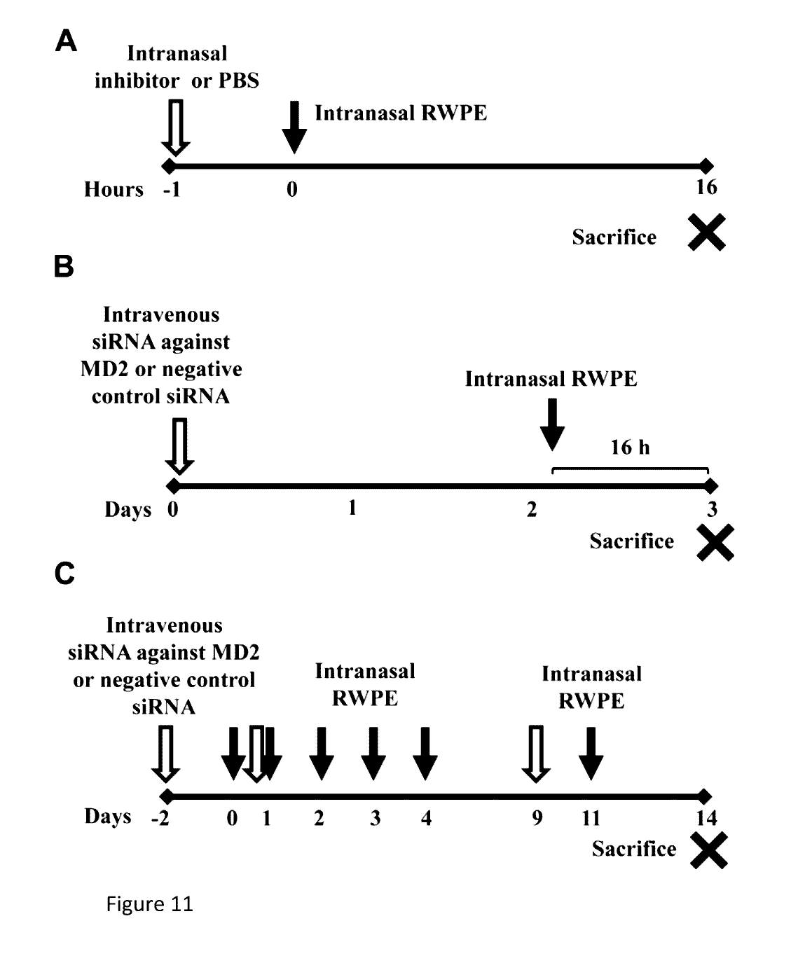

### Single-Challenge Model (See FIG. 11A)

WT mice were intranasally challenged with a single dose of RWPE or CDE (100 μg/60 μL) and euthanized after 16 hours. In additional experiments, 1 hour before RWPE challenge, WT mice were treated with or without intranasal administration of a nuclear factor κB (NF-κB) inhibitor that selectively irreversibly blocks IκBα phosphorylation with BAY 11-7082 (10 mg/kg body weight; Calbiochem, San Diego, Calif.) (13) or NEMO-binding domain binding peptide (25 per mouse, Calbiochem) (14). These treated mice were challenged with RWPE and euthanized, as described above.

Single-challenge model after single small interfering RNA treatment (see FIG. 11B). Two HPLC-purified predesigned small interfering RNAs (siRNAs) against Md2 (catalog no. s69441; Ambion Silencer, Thermo Fisher) and Tlr4 (catalog no. s75206; Ambion Silencer) and control nonspecific siRNA oligos (catalog no. 12935-100; stealth RNAi Negative Control duplexes; Ambion) were diluted in 5% glucose mixed with in vivo JET-PEI (Polyplus-transfection, New York, N.Y.). We selected the intravenous route of siRNA administration because it has been shown to suppress specific gene expression by 80% in airway epithelial cells and has minimal toxicity, unlike intranasal administration (15). Forty micrograms of each siRNA was administered to WT mice on day 0. The mice were challenged intranasally with 100 μg of RWPE or CDE on day 2 and euthanized 16 hours after challenge.

Repeated-challenge allergy model after repeated siRNA treatment (see FIG. 11C). WT mice were administered control siRNA oligos or siRNAs against Md2, as described above, on days −2, 1, and 9. These mice were administered 5 intranasal doses of RWPE (100 μg/60 μL) on days 0, 1, 2, 3, 4, and 11 to mimic chronic exposure of human subjects to RWPE (6, 16) and euthanized on day 14, as described above.

### Processing of Mouse Fluid and Tissue Samples

Total and differential bronchoalveolar lavage fluid (BALF) cell counts were performed (16). Lungs were perfused and fixed with Zn fixative (BD Biosciences, San Jose, Calif.), and sections were stained with periodic acid-Schiff for mucus staining.

Quantitative RT-PCR of Mouse Lung mRNA

RNA from mouse lung tissue was reverse transcribed and amplified by using real-time PCR in an ABI 7000 (Applied Biosystems, Foster City, Calif.). The primer sequences of MD2 were as follows: forward, 5′-AGCTCTGCAAAAAGAATAGTCATC-3′ (SEQ ID NO:7); reverse, 5′-ATAAGACTGAGGGGAACCAATG-3′ (SEQ ID NO:8). This primer were purchased from Integrated DNA Technologies (Coralville, Iowa).

### Mucin Production

Mucin production was assessed by 2 investigators who were blind to the treatment groups by using a modification of a method reported (6, 16) on a subjective scale of 0, 1, 2, 3, and 4 corresponding to none, mild, moderate, marked, or severe mucin deposition, respectively. Data were expressed as means of scores recorded by 2 blinded investigators (6, 16).

### Measurement of IL-5, IL-13, IL-33, and TSLP Levels in BALF

BALF from WT mice in a repeated-challenge allergy model were assayed for IL-5, IL-13, IL-33, and TSLP by using a DuoSet ELISA development kit (R&D Systems, Minneapolis, Minn.), according to the manufacturer's instructions.

### Measurement of RWPE-Specific Serum IgE Levels

RWPE-specific IgE levels were measured by using a previously described method (6,16). Briefly, 96-well plates were coated with 100 μg/mL RWPE protein overnight. After washing 3 times, the plates were blocked with SEA BLOCK Blocking Buffer (Pierce Biotechnology, Rockford, Ill.). Diluted sera from mice were added to the plates and incubated overnight. After washing, the plates were incubated with biotin-conjugated rat IgE (clone R35-72; BD Biosciences, San Jose, Calif.) for 2 hours at room temperature, washed, and incubated with avidin-conjugated alkaline phosphatase for 45 minutes at 4° C. After washing, fluorometric values for each well were measured after addition of AttoPhos substrate solution (Promega, Madison, Wis.).

### Studies Involving HEK 293 Cell Lines and Human Bronchial Epithelial Cells

Three HEK 293 cell lines, TCM^{Null} (TLR4^{Null}, CD14^{Null}, MD2^{Null}), TLR4^{Hi} (TLR4^{Hi}, CD14^{Null}, MD2^{Null}) and TCM^{hi} (TLR4^{Hi}, CD14^{Hi}, MD2^{Hi}; InvivoGen, San Diego, Calif.), were used. In some experiments hTERT immortalized normal human bronchial epithelial cells (HBECs) were used, as previously described (17).

NF-κB dual luciferase reporter assays. TCM^{Null}, TLR4^{Hi}, and TCM^{Hi} cells were transiently transfected with pGL4.32[luc2P/NF-κB-RE/Hygro] (Promega) vector containing multiple NF-κB response elements that drive transcription of the luciferase reporter gene luc2P (Photinus pyralis) and pRL-SV40 Renilla Luciferase Reporter Vector (Promega) by using Lipofectamine 2000 (Invitrogen, Carlsbad, Calif.). Twenty-four hours after transfection, the cells were stimulated with RWPE or PBS for 6 hours, and cell lysates were assessed with the Dual-Luciferase Reporter Assay System (Promega).

Transfection with MD2 or sham transfection. In some experiments TLR4^{Hi} cells were transfected with 2 μg of plasmid encoding MD2 (TLR4^{Hi-MD2}) (plasmid 13028; Addgene, Cambridge, Mass.) by using Lipofectamine 2000 or sham transfected with Lipofectamine 2000 (TLR4^{Hi-ST}). Twenty-four hours after transfection, the cells were stimulated with pollen allergenic extracts or CDE for 18 hours, and CXCL8 levels in cell supernatants were quantified.

Transfection with siRNA against MD2. HBECs were transfected with siRNA against human MD2 siRNA (Life Technologies, Carlsbad, Calif.) with Lipofectamine 2000 or sham transfected with Lipofectamine 2000.

Measurement of CXCL8 levels in cell supernatants. TCM^{Null}, TLR4^{Hi}, TCM^{Hi}, TLR4^{Hi-ST}, or TLR4^{Hi-MD2} cells were stimulated with RWPE, Bermuda grass, rye grass, pigweed, mountain cedar, cat dander, LPS, or Amb a 1 at 100 μg/mL for 18 hours. In some experiments HBECs, HBECs transfected with siRNA against MD2, or sham-transfected HBECs were starved for 24 hours and stimulated with RWPE, Bermuda grass, rye grass, timothy grass, pigweed, Russian thistle, cottonwood, walnut, and cat dander allergenic extracts. Cell supernatants were assayed for CXCL8.

Determination of LPS binding analysis by using fluorescence-activated cell sorting. In some experiments TLR4^{Hi}, TLR4^{Hi-MD2}, TLR4^{Hi-ST}, or TCM^{Hi} cells were incubated with 1 μg/mL LPS-Alexa Fluor 568 for 30 minutes, and cell-bound LPS-Alexa Fluor 568 was detected by using flow cytometry (BD Biosciences).

BODIPY-RWPE conjugate preparation and flow cytometry. RWPE was labeled with BODIPY FL STP Ester (B10006, Life technologies), and excess unbound dye was removed by means of column purification. TCM^{Null}, TLR4^{Hi}, TCM^{Hi}, TLR4^{Hi-ST}, and TLR4^{Hi-MD2} cells were incubated with 100 μg/mL BODIPY-RWPE conjugates for 30 minutes on ice, washed, and then subjected to flow cytometric analysis.

### Statistical Analysis

Results of the study are presented as means±SEMs. Differences between 2 groups were analyzed by using the unpaired t test. Multiple comparisons were analyzed by means of ANOVA. The software package GraphPad Prism 6 (GraphPad Software, San Diego, Calif.) was used for all data analyses and preparation of graphs. All statistical analyses considered data to be significant at a P value of less than 0.05. In all figures data are expressed as means±SEMs.

### Results

### RWPE Activates NF-κB and Stimulates Secretion of CXCL8 in TCM^{Hi} Cells

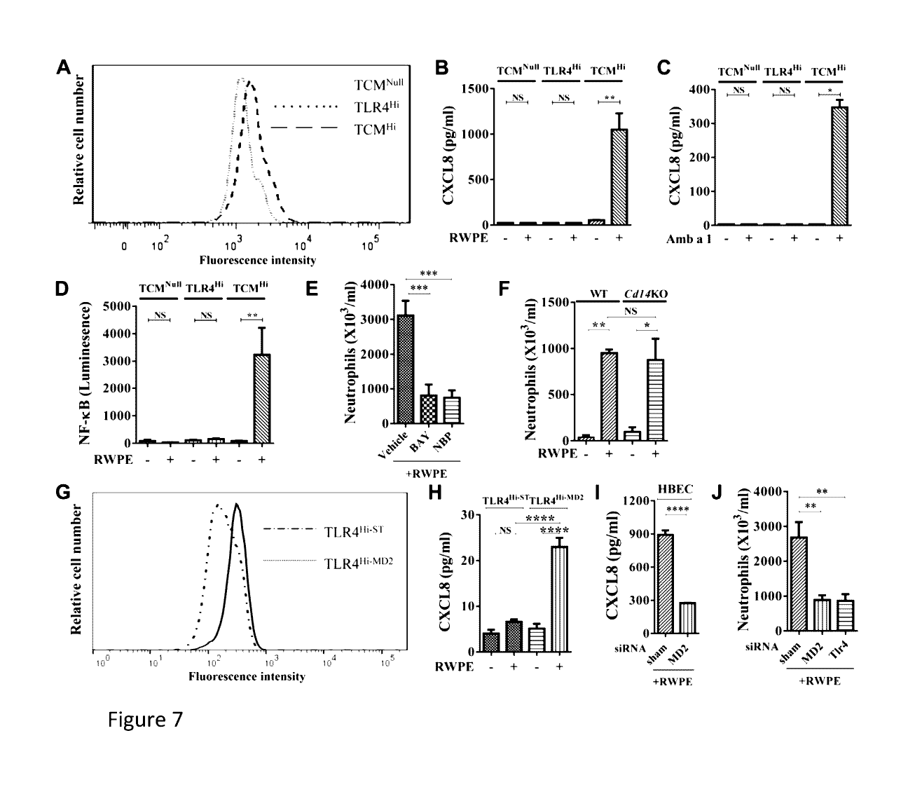

We have recently shown that a single RWPE challenge in naive mice induces TLR4- and CXCL-mediated neutrophil recruitment, and this neutrophil recruitment is critical for induction of allergic sensitization and allergic airway inflammation (6). Here we hypothesized that RWPE might require CD14, MD2, or both in addition to TLR4 to stimulate cells. To test this hypothesis, we initially selected HEK 293 cells for our studies because their inherent property of lacking TLR4, CD14, or MD2 has been extensively used in the literature to define TLR4 innate Responses (18, 19). Three types of HEK 293 cells, TCM^{Null}, TLR4^{Hi}, and TCM^{Hi}, were cultured with BODIPY-labeled RWPE for 30 minutes and subjected to fluorescence-activated cell sorting (FACS) analysis. Compared with TCM^{Null} and TLR4^{Hi} cells (<3%), TCM^{Hi} cells demonstrated higher BODIPY-RWPE staining (12.9%; FIG. 7A).

We have recently shown that RWPE challenge induces TLR4-dependent CXCL chemokine secretion (6). Building on this published observation, taken together with our observation in the current study that TCM^{Hi} cells demonstrate higher BODIPY-RWPE staining, we hypothesized that RWPE should increase CXCL chemokine levels only in TCM^{Hi} cells. Consistent with our hypothesis, RWPE induced CXCL8 secretion from TCM^{Hi} but not from TCM^{Null} cells or TLR4^{Hi} cells (FIG. 7B), validating our hypothesis. These results also suggest that RWPE requires CD14, MD2, or both in addition to TLR4 to induce CXCL chemokine secretion. Amb a 1 is a major allergen in RWPE; like RWPE, it also induced CXCL8 secretion only in TCM^{Hi} cells (FIG. 7C), indicating that a single protein in RWPE can also stimulate this pathway.

Because TLR4 signaling stimulates NF-κB activation, we examined whether RWPE requires TLR4 along with either CD14, MD2, or both to activate NF-κB. TCM^{Null}, TLR4^{Hi}, and TCM^{Hi} cells were thus transfected with an NF-κB luciferase construct. Stimulation with RWPE induced NF-κB in TCM^{Hi} but not TCM^{Null} or TLR4^{Hi} cells (FIG. 7D). These studies suggest that RWPE requires CD14, MD2, or both in addition to TLR4 to stimulate NF-κB and induce CXCL chemokine secretion. To elicit the in vivo role of NF-κB activation in mounting RWPE-induced innate inflammatory response in the lungs, the IκB kinase inhibitor BAY 11-7082 or NEMO-binding domain binding peptide was administered to mice before intranasal RWPE instillation (see FIG. 11A). Both NF-κB inhibitors decreased RWPE challenge-induced neutrophil recruitment into the airways (FIG. 7E), indicating a critical role of the NF-κB pathway in RWPE-induced innate inflammatory responses.

MD2 is the Critical Coreceptor of TLR4 that Induces RWPE-Mediated Innate Inflammatory Responses

A single-challenge model with RWPE was performed in WT and Cd14KO mice to elicit the role of CD14 in RWPE-induced innate inflammation, and mice were killed 16 hours later. RWPE challenge induced the same level of neutrophil recruitment in BALF from Cd14KO as WT mice (FIG. 7F), indicating that CD14 is not an essential coreceptor for TLR4 to induce RWPE-induced innate response in the lungs. TLR4^{Hi} cells were sham transfected (TLR4^{Hi-ST}) or transfected with a plasmid to overexpress MD2 (TLR4^{Hi-MD2}) to validate the role of MD2 in pollen-induced CXCL chemokine synthesis. FACS analysis of TLR4^{Hi-ST} and TLR4^{Hi-MD2} cultured with BODIPY-labeled RWPE demonstrated higher BODIPY-RWPE staining in TLR4^{Hi-MD2} than TLR4^{Hi-ST} cells (FIG. 7G). Stimulating TLR4^{Hi} cells (data not shown) and TLR4^{Hi-ST} cells with RWPE did not induce CXCL8 secretion (FIG. 7H). By contrast, stimulating TLR4^{Hi-MD2} cells with RWPE induced CXCL8 secretion (FIG. 7H).

Next, we sought to determine the relevance of these data from HEK 293 cells to HBECs (17). Stimulation of HBECs with RWPE induced CXCL8 secretion (FIG. 7I). Suppression of MD2 in HBECs by siRNA inhibited RWPE-induced CXCL8 secretion (FIG. 7I). To test the in vivo relevance of MD2 in stimulating the RWPE-induced innate immune response, we first tested the efficacy of siRNA against Md2 or Tlr4 in suppressing lung expression of these genes. siRNA against Md2 or Tlr4 (see FIG. 11B) suppressed Md2 or Tlr4 mRNA expression in the lungs by 60% or 71%, respectively (data not shown). Similar to our previously reported observations in Tlr4KO mice (6), suppression of Tlr4 mRNA by means of siRNA administration inhibited RWPE-induced neutrophil recruitment (FIG. 7J), validating this strategy as a tool to test the in vivo role of MD2. Consistent with our studies in HEK cells, in vivo suppression of Md2 by siRNA administration (see FIG. 11B) reduced RWPE-induced neutrophil recruitment (FIG. 7J). These studies indicate an important role of MD2 in mediating the innate immune responses to RWPE.

Pollen Extracts from Diverse Plant Families Use MD2 as a Critical Coreceptor of TLR4 to Stimulate CXCL Chemokine Secretion

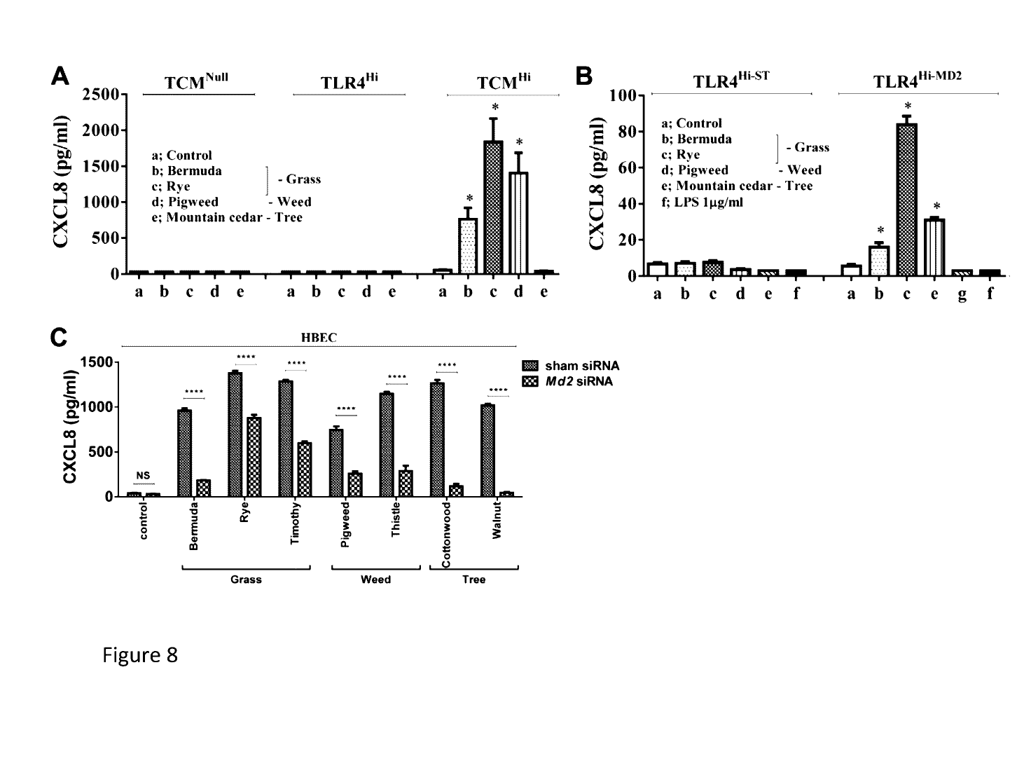

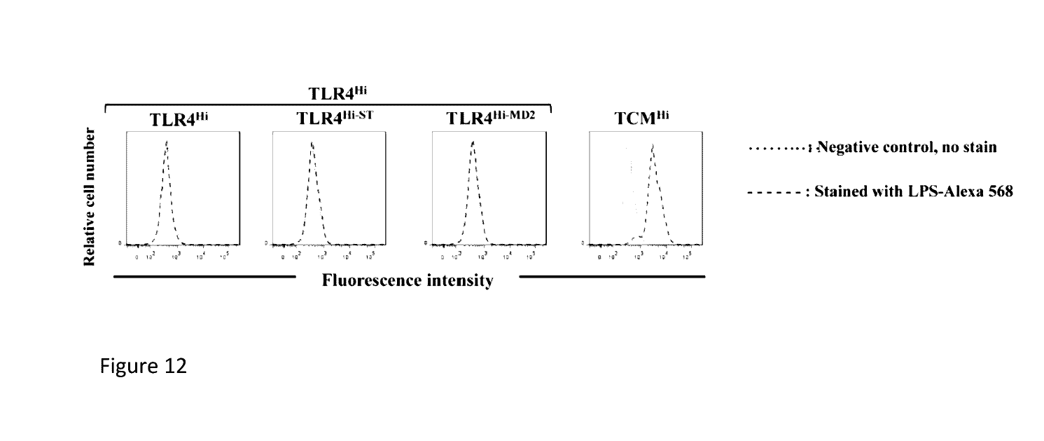

Next, we examined the broader role of TLR4, CD14, and MD2 in stimulating CXCL chemokine in response to culture with allergenic pollen extracts from diverse plant families. Pollen allergenic extracts belonging to diverse families, grasses (Bermuda and rye), and weeds (pigweed) induced CXCL8 secretion from TCM^{Hi} but not TCM^{Null} or TLR4^{Hi} cells (FIG. 8A). By contrast, 10 replicates stimulated with tree pollen extract (mountain cedar) did not induce CXCL8 secretion, demonstrating the specificity of innate immune recognition of allergenic extracts. Like RWPE, stimulating TLR4^{Hi} (data not shown) and TLR4^{Hi-ST} cells with these pollen allergenic extracts did not induce CXCL8 secretion (FIG. 8B). By contrast, stimulating TLR4^{Hi-MD2} cells with Bermuda, rye, firebush, and pigweed, but not mountain cedar, induced CXCL8 secretion (FIG. 8B). This pattern of CXCL8 secretion was similar to that seen in TCM^{Hi} cells (FIG. 8A), demonstrating the reproducibility and specificity of innate recognition of diverse allergens. By contrast, a very high concentration of LPS (1 μg/mL) did not bind (see FIG. 12) or induce CXCL8 secretion (FIG. 8B) from TLR4^{Hi-MD2} cells. Together, these observations indicate that diverse pollen allergenic extracts require MD2 in addition to TLR4 to mount innate immune responses characterized by secretion of CXCL chemokines and use a mechanism that is distinct from LPS to induce this secretion.

Next, we sought to validate our results by using HBECs (17). Stimulation with diverse pollen allergenic extracts, such as Bermuda, rye, timothy, pigweed, Russian thistle, eastern cottonwood, and black walnut, induced CXCL8 secretion (FIG. 8C). Suppression of MD2 by siRNA inhibited pollen allergenic extract-induced CXCL8 secretion (FIG. 8C). Firebush and mountain cedar did not induce CX CL8 secretion from HBECs (data not shown), indicating the specificity of innate immune recognition of these pollen extracts.

### CDE Requires MD2 in Addition to TLR4 to Induce CXCL8 Secretion and Neutrophil Recruitment

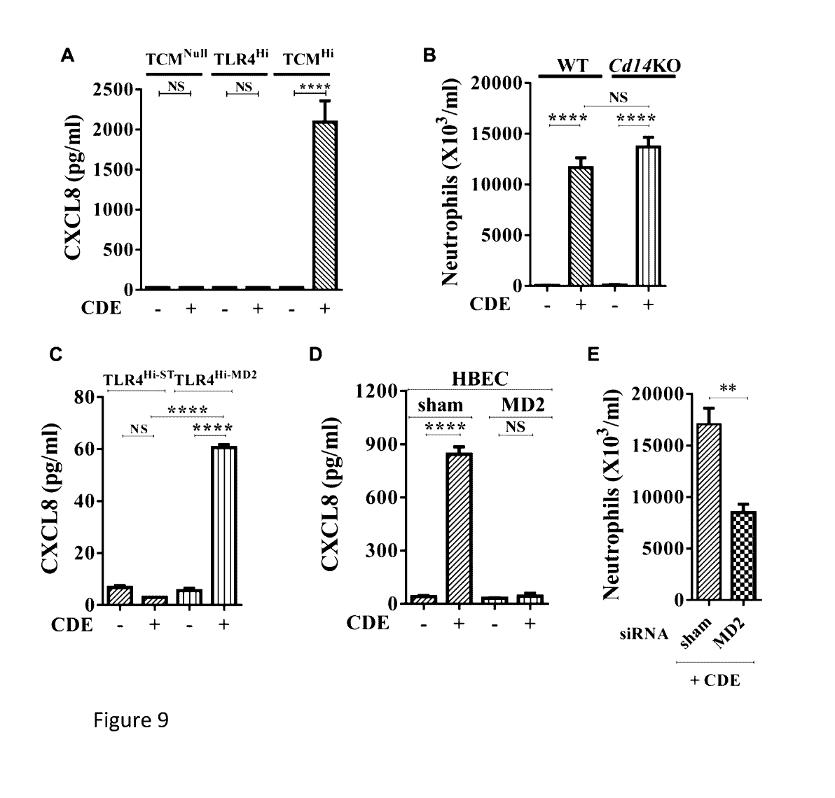

Next, we examined whether CDE, an allergenic extract completely unrelated to pollen extracts, stimulates an MD2-dependent innate immune response. Similar to RWPE and other pollen allergens, CDE induced CXCL8 secretion in TCM^{Hi} but not TCM^{Null} or TLR4^{Hi} cells (FIG. 9A). Intranasal challenge with CDE induced the same level of neutrophil recruitment in BALF from Cd14KO as from WT mice (FIG. 9B). These results indicated that CD14 is not an essential coreceptor for TLR4 for CDE to induce innate recruitment of neutrophils. Stimulating TLR4^{Hi-ST} cells with CDE did not induce CXCL8 secretion (FIG. 9C). By contrast, stimulating TLR4^{Hi-MD2} cells with CDE induced CXCL8 secretion (FIG. 9C). Likewise, stimulation of HBECs with CDE induced CXCL8 secretion (FIG. 9D), and suppression of MD2 by siRNA inhibited the CDE-induced CXCL8 secretion from these cells (FIG. 9D). siRNA suppression of lung Md2 before an intranasal single-challenge model with CDE (see FIG. 11B) inhibited CDE-induced neutrophil recruitment (FIG. 9E). Together, these studies indicate that CDE requires MD2 in addition to TLR4 to induce CXCL chemokine secretion and induce recruitment of neutrophils to the airways.

### MD2 Facilitates RWPE-Induced Allergic Sensitization and Allergic Airway Inflammation

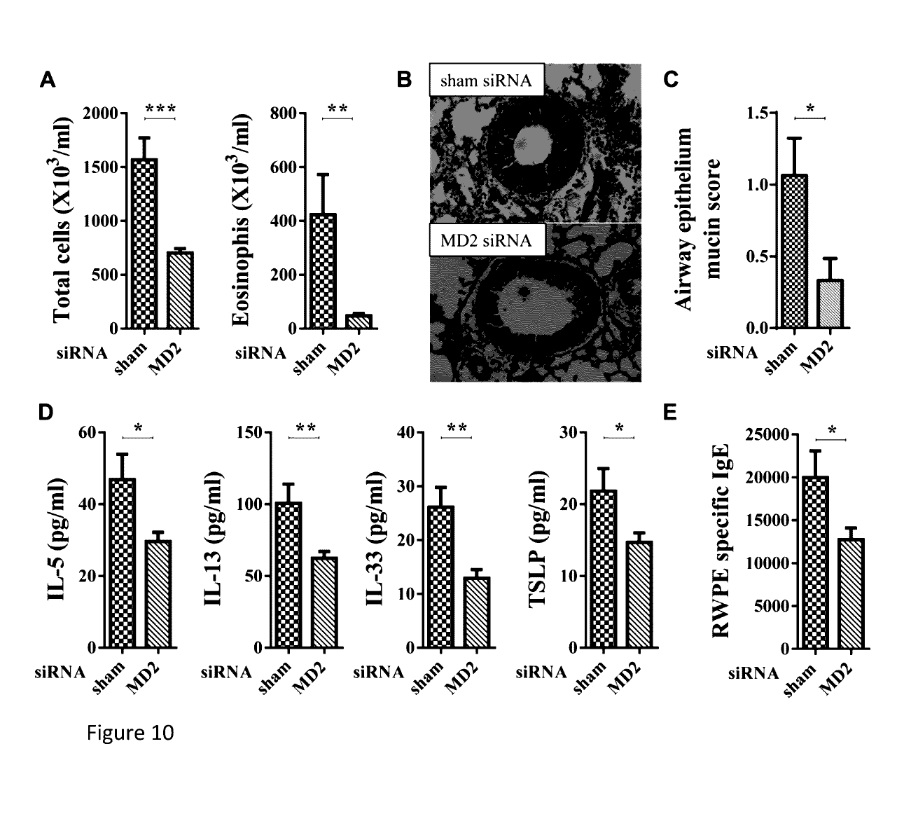

Building on our observation that MD2 mediates pollen and CDE-induced CXCL chemokine secretion and neutrophil recruitment, taken together with our recent report that RWPE challenge-induced TLR4- and CXCL-mediated neutrophil recruitment is critical for induction of allergic sensitization and allergic airway inflammation (6), we hypothesized that MD2 is important for inducing allergic sensitization and allergic inflammation to pollen and cat dander. To test this hypothesis, we used RWPE as a model system. An siRNA against Md2 was administered to WT mice before and during RWPE instillations in the repeated-challenge model (see FIG. 11C). Compared with administration of control siRNA, administration of siRNA against Md2 strongly attenuated RWPE-induced allergic sensitization and allergic airway inflammation. This inhibition consisted of a decrease in recruited eosinophil and total inflammatory cell counts (FIG. 10A); a decrease in the accumulation of mucin in epithelial cells (FIGS. 10B and C); a decrease in IL-5, IL-13, IL-33, and TSLP levels in BALF (FIG. 10D); and a decrease in RWPE-specific serum IgE levels (FIG. 10E). These observations indicate that MD2 facilitates RWPE-induced allergic sensitization and allergic airway inflammation.

### Discussion

Neutrophils have long been viewed as terminally differentiated cells that clear extracellular pathogens. However, a growing body of literature indicates that neutrophils have numerous additional effects that regulate innate and adaptive immune responses (20, 21). Neutrophil recruitment is a hallmark of innate immune responses (22), and innate recruitment of neutrophil to the skin through leukotriene B₄ is critical for induction of subsequent allergic skin inflammation (22, 23). Recently, we reported that RWPE challenge induces CXCR2- and TLR4-dependent innate recruitment of activated neutrophils to the airways and that these recruited neutrophils are critical for induction of allergic sensitization and allergic airway inflammation (6). In the present study we extend our earlier observations by demonstrating a broad role of MD2 in induction of innate and allergic airway inflammation and allergic sensitization by CDE, RWPE, and extracts of pollens from grasses, weeds, and trees (6).

Similar to the present study, several earlier studies reported neutrophil influx in response to allergen challenge in asthmatic patients (24-26). One interpretation of neutrophil recruitment after allergen exposure is that endotoxin contamination of allergenic extracts or stimulation of TLR4/MD2 signaling by allergen-LPS complex could explain neutrophil recruitment after pulmonary allergen challenge in those studies and in the present study (27, 28). However, several lines of evidence strongly indicate that endotoxin contamination/signaling cannot explain the observations in the present study. First, in the present study all allergenic extracts had very low (<0.1 pg of LPS/1 μg of allergen protein) endotoxin levels, well below those of the no-azide low-endotoxin neutralizing antibodies used in numerous cell culture and in vivo studies (BD Biosciences). Second, we demonstrate in this article that RWPE and CDE use a CD14-independent pathway to induce an innate neutrophilic airway inflammation, distinguishing it from LPS-induced inflammatory response. Third, using TLR4^{Hi-MD2} cells that do not bind or respond to very high concentrations of LPS, we show that RWPE, CDE, and other allergenic extracts induce CXCL chemokine secretion. Finally, we have reported that repeated RWPE challenges together with passive transfer of neutrophils from donor mice to Tlr4KO recipient mice (that lack the major receptor to respond to LPS) overcomes the blockade of RWPE-induced allergic sensitization and airway inflammation observed in TLR4-null mice (6). Together, these data indicate that allergenic extracts directly induce CD14-independent and MD2- and TLR4-dependent innate immune responses in the lungs, possibly by using a mechanism similar to HIV-1 Tat, which binds TLR4-MD2 and stimulates an innate cytokine response independent of CD14 (29). This MD2/TLR4-induced innate neutrophil recruitment facilitates allergic sensitization and allergic airway inflammation (6, 23).

In the present study, even though the pattern of stimulation of CXCL8 by pollen extracts in TCM^{Hi} cells was similar to that in TLR4^{Hi-MD2} cells, CXCL8 chemokine levels were 20- to 50-fold higher in TCM^{Hi} cells. This striking difference in pollen extract-induced CXCL8 secretion between the 2 cell types might reflect damage to TLR4^{Hi-MD2} cells during transient transfection with plasmid DNA and lipofection versus no damage in stably transfected TCM^{Hi} cells. Alternatively, transient transfection of TLR4^{Hi-MD2} cells might have induced relatively low levels of MD2 expression compared with long-term stably transfected and selected TCM^{Hi} cells. We have recently reported that BAL neutrophil numbers distinguish controlled asthma from uncontrolled asthma and correlate inversely with FEV₁ (30). Likewise, the dominance of neutrophils has been reported in the airways of patients with severe asthma (31) and those with sudden-onset fatal asthma (32). Future studies will have to investigate whether stimulation of MD2-TLR4 signaling by allergens, as elucidated in the present study, provides a molecular mechanism basis of neutrophilic inflammation in the earlier studies. If proved correct, additional carefully conducted mechanistic studies will be required to determine whether these recruited neutrophils contribute to severe asthma and sudden-onset fatal asthma by stimulating allergic sensitization and allergic inflammation (6, 23). Our data indicate that diverse allergenic extracts require both MD2 and TLR4 to induce innate and allergic inflammation (6), distinguishing pollen extracts and CDE from several TLR4 ligands, such as lipid A (33), taxol (34), nickel, and cobalt, that stimulate TLR4 without MD2 (35). Der p 2, the major component of house dust mite, shows structural homology with MD2 and enhances allergic inflammation by facilitating TLR4 signaling (11, 36). Because Amb a 1 has no structural similarity with MD2 but can stimulate MD2-dependent CXCL chemokine secretion, it uses a mechanism that is distinct from the structural mimicry of MD2 used by house dust mites (11).

MD2 has been reported as one of 7 single nucleotide polymorphisms in 6 genes associated with asthma (37). Our data suggest that future studies should investigate whether MD2 inhibitor or antagonists, such as curcumin (38), prenylated flavonoids (39), rifampin (40), and eritoran (41), can inhibit allergic disorders, particularly those induced by ragweed, Bermuda, rye, timothy, pigweed, Russian thistle, cottonwood, and CDEs. Future studies should investigate whether use of specific inhibitors of the MD2 pathway could be a strategy to prevent neutrophil-dominant forms of asthma, such as severe asthma (31).

Clinical implications: Blocking MD2 might be a novel strategy of inhibiting allergic sensitization and allergic inflammation induced by common allergenic pollens and cat dander.

### REFERENCES FOR EXAMPLE 2

1. Sporik et al., Thorax 1999; 54:675-80.
2. Salo et al., J Allergy Clin Immunol 2011; 127:1226-35.e7.
3. Salo et al., J Allergy Clin Immunol 2014; 134:350-9.
4. Li et al., Allergy Clin Immunol 2011; 128:1318-25.e2.
5. Shalaby et al., J Immunol 2013; 191:922-33.
6. Hosoki et al., Am J Respir Cell Mol Biol 2015.
7. Shimazu et al., J Exp Med 1999; 189:1777-82.
8. Viriyakosol et al., J Biol Chem 2001; 276:38044-51.
9. da Silva Correia et al., J Biol Chem 2001; 276:21129-35.
10. Nagai et al., Nat Immunol 2002; 3:667-72.
11. Trompette et al., Nature 2009; 457:585-8.
12. Boldogh et al., J Clin Invest 2005; 115:2169-79.
13. Alvira et al., Am J Respir Crit Care Med 2007; 175:805-15.
14. Haeberle et al., J Virol 2004; 78:2232-41.
15. McCaskill et al., Mol Ther Nucleic Acids 2013; 2:e96.
16. Wild et al., J Immunol 2000; 164:2701-10.
17. Rada et al., Am J Respir Cell Mol Biol 2014; 50:125-34.
18. Erridge et al., J Biol Chem 2008; 283:24748-59.
19. Junker et al., J Exp Med 2012; 209:2395-408.
20. Mantovani et al., Nat Rev Immunol 2011; 11:519-31.
21. Mocsai, J Exp Med 2013; 210:1283-99.
22. Lammermann et al., Nature 2013; 498:371-5.
23. Oyoshi et al., Immunity 2012; 37:747-58.
24. Lommatzsch et al., J Allergy Clin Immunol 2006; 118:91-7.
25. Hoskins et al., Am J Respir Crit Care Med 2013; 187:34-41.
26. Metzger et al., Chest 1986; 89:477-83.
27. Hunt et al., Am J Respir Crit Care Med 1994; 149:1471-5.
28. Herre et al., J Immunol 2013; 191:1529-35.
29. Ben Haij et al., Retrovirology 2013; 10:123.
30. Hosoki et al., PLoS One 2015; 10:e0126035.
31. Jatakanon et al., Am J Respir Crit Care Med 1999; 160:1532-9.
32. Sur et al., Am Rev Respir Dis 1993; 148:713-9.
33. Lien et al., J Clin Invest 2000; 105:497-504.
34. Hajjar et al., Nat Immunol 2002; 3:354-9.
35. Raghavan et al., EMBO Rep 2012; 13:1109-15.
36. Hammad et al., Nat Med 2009; 15:410-6.
37. Sharma et al., Pediatr Allergy Immunol 2012; 23:315-23.
38. Wang et al., Mol Biosyst 2015; 11:1933-8.
39. Peluso et al., Planta Med 2010; 76:1536-43.
40. Wang et al., FASEB J 2013; 27:2713-22.
41. Shirey et al., Nature 2013; 497:498-502.

### Example 3

### Introduction

Cat dander is one of the most common indoor allergens (1-3). The NHANES studies identified cat dander as a major allergen that induces allergic sensitization and allergic diseases (1, 3). The prevalence of sensitization to cat is 9-16% among adults (4-7) and 5-19% in children (8-11). Sensitization to cat dander in children is a risk factor for the diagnosis of asthma (12, 13), asthma severity (14) and hospitalization rate due to asthma attacks (15). Likewise, sensitization to cat dander in adults is also a risk factor for the current asthma (16). Exposure to cat dander increases airway hyperresponsiveness (17) and induces small airway obstruction (18). Environmental measures such as HEPA filters are not effective in removing cat dander (19). Omalizumab ameliorates the severity of acute airway obstruction and symptoms caused by exposure to cat allergen (20), indicating the cat-dander-specific IgE plays an important role morbidity from exposure to cats. However, the innate immune responses that are triggered following exposure to cat dander, and the contribution of these innate responses to allergic sensitization and disease have not been critically evaluated.

Exposure of the airways to cat dander or pollen extract rapidly stimulates recruitment of neutrophils into the airways (21-26). Neutrophil recruitment is the hallmark of a rapid innate immune responses (27). The role of neutrophils in fighting bacterial infections is well established. However, recent studies have established additional roles for this cell in modulating adaptive immune response (28). One study reported that Leukotriene B4 (LTB4) played a critical role in stimulating neutrophil recruitment to the skin, and these recruited neutrophils stimulated induction of allergic cutaneous inflammation (28). Several recent studies indicate that pollen-induced innate immune responses contribute to the initiation of airway allergic disorders. Toll-like receptor 4 (TLR4)-mediated thymic stromal lymphopoietin (TSLP)/OX40 ligand (OX40L)/OX40 signaling is crucial in allergic conjunctivitis (29). TLR4 contributes birch pollen-induced allergic airway inflammation (30). We reported that exposure of the airway to ragweed pollen extract rapidly stimulates TLR4 and CXCR2-dependent recruitment of neutrophils to the airways, and these recruited neutrophils stimulate induction of allergic sensitization to ragweed pollen extract and allergic airway inflammation (Example 1). We subsequently demonstrated that TLR4 requires an additional molecule, myeloid differentiation factor 2 (MD2), to mount and innate neutrophilic inflammatory response to ragweed pollen extract and stimulate allergic sensitization and allergic airway inflammation (Example 2). We further reported that cat dander also requires MD2 to stimulate an innate neutrophilic inflammatory response (Example 2).

The TLR4/MD2 signaling pathway utilizes adaptor molecules Myd88 or TRIF to generate proinflammatory cytokines (31, 32). Controversy exists about role of MyD88 in allergic diseases, and its role is an area of active research. TLR4 ligands like lipopolysaccharide (LPS) that signal through MyD88 impairs the ability of OVA to induce airway eosinophilia, type-2 cytokines secretion, airway hyper-reactivity, mucus hyper production and serum levels of IgE (33). Furthermore, collateral priming of naïve CD4+T cells to OVA-specific Th2 cells in the presence of OVA-transgenic T-cells does not require Myd88 (34). By contrast, challenge with short ragweed pollen in sensitized mice induces TSLP secretion and allergic inflammation more effectively in the presence of an intact MyD88 signaling pathway (29). Likewise, subtilisin, a potent serine protease used in detergent industry, induces airway allergic inflammation that is dependent of Myd88 (35). Furthermore, Aspergillus fumigatus-induced atopic dermatitis is dependent on MyD88 signaling (36). By contrast, very little is known about the TLR-receptors in cat dander-induced innate immune and allergic immune responses in the lungs. One study reported that cat dander protein Fel d 1 does not bind TLR4/MD2 but can bind LPS (37). This study did not evaluate the role of TLR in cat dander-induced inflammation. There are no report that identifies the critical TLR adaptor that regulates cat dander-induced innate and allergic responses, nor genes that are controlled by this key adaptor. The objectives of the present study were to address these critical gaps in knowledge in cat dander extract (CDE)-induced innate and allergic gene expression and inflammation.

Abbreviations used: BALF, bronchoalveolar lavage fluid; CDE, cat dander extract; HDM, House dust mite; LTB4, Leukotriene B4; LPS, lipopolysaccharide; MD2, myeloid differentiation protein-2; ROS, Reactive oxygen species; RWPE, Ragweed pollen extract; TLR4, Toll-like receptor 4; and TSLP, Thymic stromal lymphopoietin

### Methods

### Mice.

Eight- to 12-wk-old male WT mice (C57BL/10SNJ), WT mice (C57BL/6J), Tlr4KO mice (C57BL/10ScNJ), Tlr2KO mice (B6.129-Tlr2^{tm1Kir}/J), Myd88 KO mice (B6.129P2(SJL)-Myd88^{tm1.1Defr}/J), and TrifKO mice (C57BL/6J-Ticam1^{Lps2}/J) from Jackson Laboratory (Bar Harbor, Me.) were used for our studies. All mice were maintained in a pathogen-free environment throughout the experiments at the University of Texas Medical Branch (Galveston, Tex.). Animal experiments were approved by the UTMB Animal Care and Use Committee and performed according to the NIH Guide.

### Allergenic Extracts.

Lyophilized CDE, rye grass pollen extract (RyePE), ragweed grass pollen extract (RWPE), and cotton wood extract was purchased from Greer Labs (Lenoir, N.C.). In our previous studies (Examples 1 and 2), we have demonstrated that there was very low amount of endotoxin in each allergen (<0.1 pg LPS/1 μg allergen protein) by using LAL chromogenic endotoxin quantitation kit (Thermo Scientific, Hudson, N.H.).

### Protocols Used for Animal Studies

Mice were sedated with low dose intraperitoneal xylazine-ketamine anesthetic mixture for intranasal sensitization or challenge and sacrificed by lethal intraperitoneal xylazine/ketamine overdose (21).
Single-challenge model. WT mice, Tlr4KO mice, Tlr2KO mice, Myd88KO mice, and Trif/KO mice were intranasally challenged with a single dose of CDE, RWPE, RYE, or cotton wood (100 μg/60 μl), and sacrificed after 2 or 16 hours-post challenge.
Repeated-challenge model. WT mice or Myd88KO mice were sensitized by five intranasal doses of cat dander extract (100 μg/60 μl) on days 0, 1, 2, 3, and 4. These mice were challenged with intranasal PBS or cat dander extract (100 μg/60 μl) on day 11. Sacrifice was performed after 2, 4, 16, or 72 hours-post challenge (38).

### Processing of Mouse Fluid and Tissue Samples

Bronchoalveolar lavage fluid (BALF) and other samples for biochemical and histological examinations were taken as previously described (39). Briefly, to collect the BALF, after lethal dose of anesthesia, trachea of mice were cannulated, and lungs were lavaged with two 0.7-ml aliquots of ice-cold Dulbecco's PBS (Corning, Manassas, Va.). Mice were thoracotomized and the blood in pulmonary circulation flushed with saline, and the lungs were excised. Lungs were perfused and fixed with Zn fixative (BD Biosciences, San Jose, Calif.) for at least 24 hours and then embedded in paraffin, sectioned at a thickness of 4 um, and stained with hematoxilin/eosin (HE) for morphology or periodic acid-Schiff (PAS) for mucus staining.

### Total and Differential Counts in BALF

Total cell counts in BALF were determined from an aliquot of the cell suspension. For differential cell counting, the BALF cells were centrifuged using a cytospin and stained with modified Wright Giemsa stain. The number of eosinophils, neutrophils, lymphocytes, and macrophages was determined by performing a differential count on at least 500 cells/slide of a cytocentrifuge preparation (38, 39).

### Measurement of IL-5, IL-13, TSLP, and IL-33 in BALF

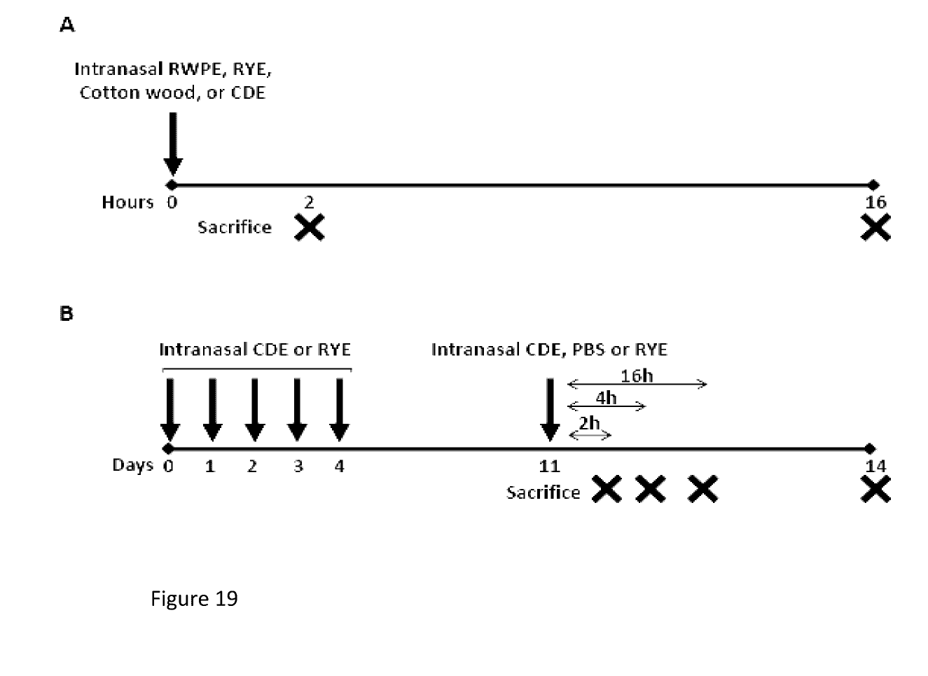

The BALF from the WT mice and Myd88KO mice repeatedly challenged with CD (FIG. 19A) and assayed for IL-5, IL-13, TSLP, and IL-33 using a DuoSet ELISA development kit (R&D Systems, Minneapolis, Minn.) according to the manufacturer's instructions.

### Epithelial Mucin Score

Two investigators who were blinded to the treatment groups assessed mucin production using a minor modification of a method reported (38) on a subjective scale of 0, 1, 2, 3, and 4 corresponding to none, mild, moderate, marked, or severe mucin deposition, respectively. The data were expressed as mean of score recorded by two blinded investigators.

### Measurement of Total IgE and Cat Dander-Specific IgE

96 wells plates were coated with 10 μg/ml of cat dander protein or rat anti-mouse IgE (BD Biosciences, San Jose, Calif.) for 2 hours at room temperature and blocked for 1 hour with sea block buffer. Plates were applied with serum overnight. After washing, biotin-conjugated rat IgE anti-mouse I IgE (BD) followed by avidin-alkaline phosphatase were added. Fluorescence intensity were analyzed using AttoPhos Substrate Solution (Promega, Madison, Wis.).

### PCR Array Analysis

Total RNA was extracted from mouse lung using an RNeasy mini kit according to the manufacturer's instructions (Qiagen, Valencia, Calif.). cDNA was synthesized with cDNA Synthesis Using the RT² First Strand Kit (Qiagen, Valencia, Calif.). Amplification by real-time PCR was performed on an ABI 7000 Sequence Detection System (Applied Biosystems, Foster City, Calif.) using SYBR Green PCR Master Mix Kit (Qiagen, Valencia, Calif.). RT² Profiler PCR Array for Mouse allergy and asthma Analysis or Mouse innate and adaptive immune responses Analysis were performed using SYBR Green master mix according to the manufacturer's protocol (catalog number PAMM-067Z and PAMM-052Z, respectively; SABiosciences, Valencia, Calif.). The results were analyzed by the PANTHER (Protein Analysis through Evolutionary Relationships, version 9.0) classification system (available on the World Wide Web at pantherdb.org) for signaling pathways and protein classes. The figure of hierarchical clusters was constructed with GENE-E software from Broad Institute (available on the World Wide Web at broadinstitute.org/cancer/software/GENE-E/). In the figure of hierarchical clustering analysis, red represents expression statistically significant increase in expression, whereas green shows significant reduction in expression.

### Statistical Analysis.

Results of the study are presented as means±SEM. Comparison of two groups were analyzed by unpaired t-test. Multiple comparisons were analyzed by ANOVA. The software package GraphPad Prism 6 (GraphPad Software, San Diego, Calif.) was used for all data analyses and preparation of graphs. All statistical analyses considered data significant at p<0.05.

### Results

### Cat Dander Extract Requires Myd88 to Recruit Neutrophil to the Airway

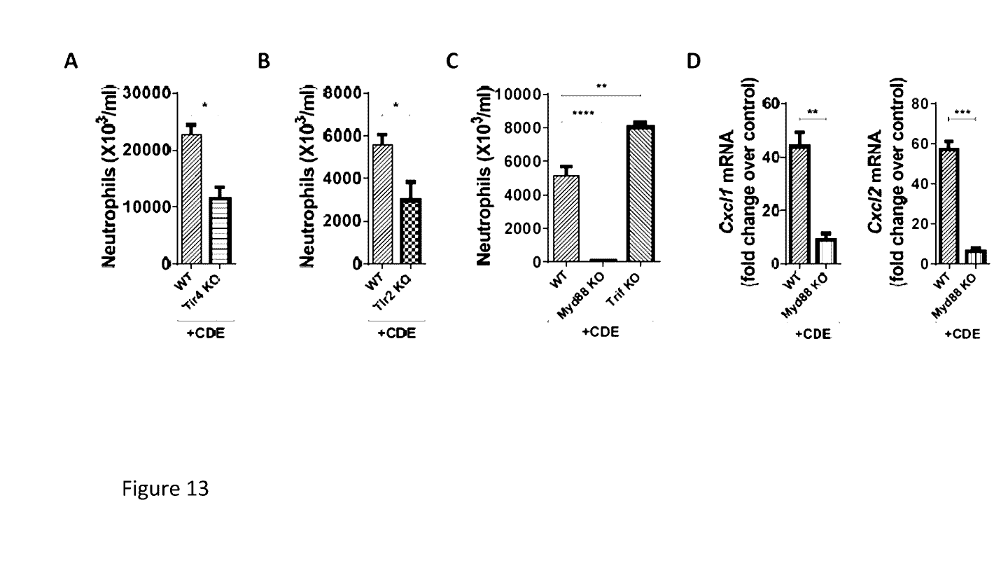

We have previously reported that RWPE induced TLR4 and CXCL1/2 secretion mediated neutrophilic recruitment (Example 1). We have recently reported that CDE utilizes MD2 to recruit neutrophils to the airways (Example 2). Since MD2 is a TLR4-coreceptor, these data strongly suggest a role of TLR4 in CDE-induced innate neutrophil recruitment. As predicted from our earlier data, a single intranasal challenge (FIG. 19A) with CDE recruited less neutrophils to the airway in Tlr4 KO mice than WT mice (FIG. 13A). Unexpectedly, in contrast to ragweed pollen extract-induced innate neutrophil recruitment that is totally dependent on TLR4 (Example 1), disruption of TLR4 only reduced CDE-induced innate neutrophil recruitment by about 50% (FIG. 13A) compared to WT mice. These data implied that a second TLR also participates in CDE-induced innate neutrophil recruitment. We hypothesized that TLR2 may be that second receptor. Consistent with our hypothesis, a single intranasal challenge with CDE recruited 50% less neutrophils to the airway in Tlr2 KO mice than WT mice (FIG. 13B). Together these data indicate that CDE-mediated innate neutrophil recruitment is dependent on both TLR2 and TLR4.

Since the common adaptor shared by TLR2 and TLR4 is Myd88, we hypothesized that Myd88 might comprehensively regulate CDE-induced innate neutrophil recruitment. Consistent with our hypothesis, neutrophil recruitment by a single challenge with CDE was abolished in Myd88 KO mice (FIG. 13C). By contrast, cat dander extract-induced innate neutrophil recruitment in TrifKO mice was significantly higher than WT mice (FIG. 13C). As expected from our earlier report (Example 2), challenge of naïve WT mice with CDE stimulated an innate response that increased lung mRNA expression of neutrophil-recruiting CXC chemokines Cxcl1 and Cxcl2 (FIG. 13D). Disruption of Myd88 abrogated this increase in mRNA expression of Cxcl1 and Cxcl2 (FIG. 13D). Taken together with our earlier reports (Examples 1 and 2), these results suggest that Myd88, and not TRIF, is the critical adaptor that innately increases CXC chemokines in the lungs and recruits neutrophils.

### Identification of CDE-Induced Innate Immune Response Genes in the Lungs

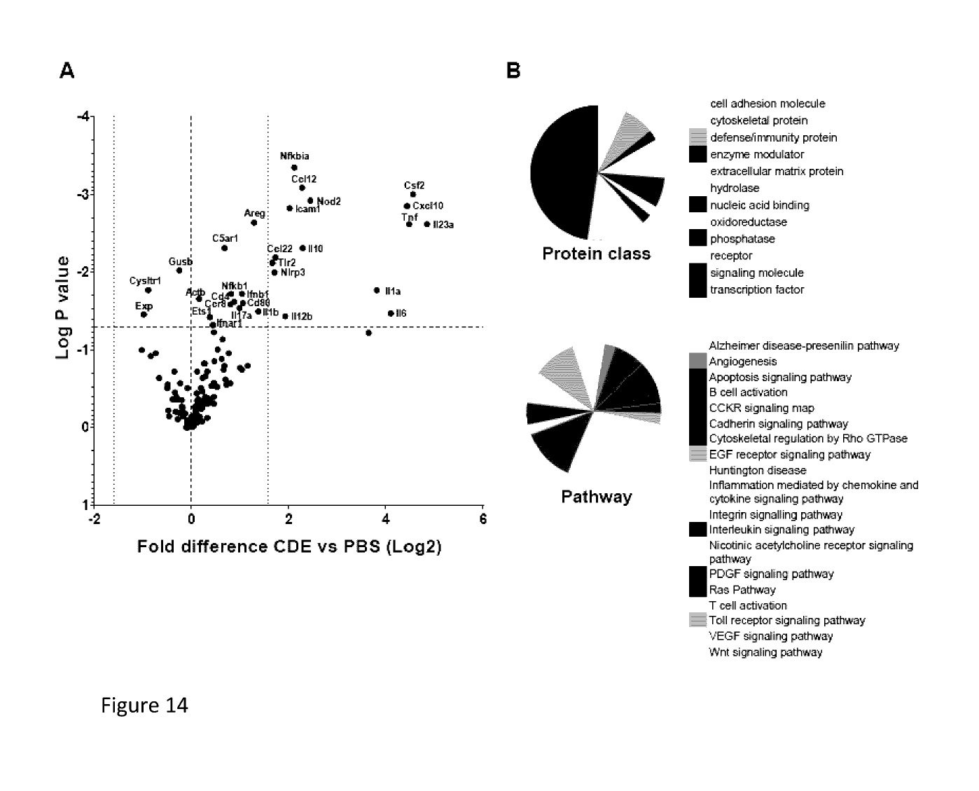

Building on our observation that MyD88 regulates CDE-mediated cxcl1 and cxcl2 mRNA expression associated neutrophil recruitment, we hypothesized that Myd88 also stimulates other innate and allergic associated gene. To test this hypothesis, we performed PCR arrays of 155 innate and allergic-inflammation associated genes in the lungs to examine the CDE challenge-induced expression. A single CDE challenge in WT mice significantly increased lung mRNA expression of 27 genes: Actb, Areg, C5ar1, Ccl12, Ccl22, Ccr8, Cd4, Cd80, Csf2, Cxcl10, Ets1, Icam1, Ifnar1, Ifnb1, Il10, Il12b, Il17a, Il1a, Il1b, Il23a, Il6, Nfkb1, Nfkbia, Nlrp3, Nod2, Tlr2, and Tnf and decreased lung mRNA expression of three genes: Cys1tr1, Epx, and Gusb (FIG. 14A). None of the other 125 genes were increased or decreased.

The PANTHER (Protein ANalysis THrough Evolutionary Relationships) Classification System is open access system supported by National Institutes if General Medicine to classify genes and proteins and facilitate high-throughput analysis. Panther analysis revealed that there were 12 protein class and 19 signaling pathway involved with these mRNA changes in single challenge model (FIG. 14B). Protein classes included cell adhesion molecule, cytoskeletal protein, defense/immunity protein, enzyme modulator, extracellular matrix protein, hydrolase, nucleic acid binding, oxidoreductase, phosphatase, receptor, signaling molecule, and transcription factor (FIG. 14B). Pathway included Alzheimer diseases-presenilin pathway, Angiogenesis, Apoptosis signaling pathway, B cell activation, CCKR signaling map, Cadherin signaling pathway, Cytoskeletal regulation by Rho GTPase, EGF receptor signaling pathway, Huntington disease, Inflammation mediated by chemokine and cytokine signaling pathway, Interleukin signaling pathway, Nicotinic acetylcholine receptor signaling pathway, PDGF signaling pathway, Ras Pathway, T cell activation, Toll receptor signaling pathway, VEGF signaling pathway, and Wnt signaling pathway (FIG. 14B).

### Identification of Myd88-Dependent CDE-Induced Innate Immune Response Genes in the Lungs

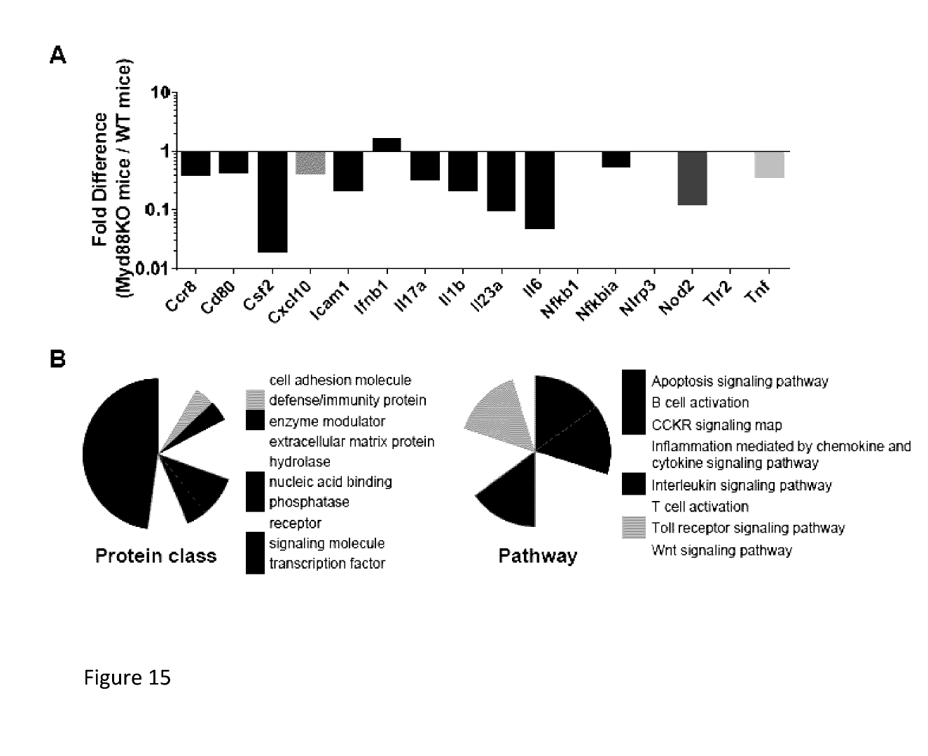

To define the role of Myd88 in innate immune response, we compared gene expression after 2 hours-post single CDE challenge in WT mice to that induced in Myd88 KO mice. Disruption of MyD88 decreased the lung mRNA expression of Ccr8, Cd80, Csf2, Cxcl10, Icam1, Il17a, Il1b, Il23a, Il6, Nfkb1, Nfkbia, Nlrp3, Nod2, Tlr2, and Tnf (FIG. 15A), and increased the lung mRNA expression of Ifnb1 (FIG. 15A).

Panther analysis revealed that there were 10 protein class and 8 signaling pathway involved with Myd88-mediated mRNA changes in the lungs (FIG. 15B): Protein classes included cell adhesion molecule, defense/immunity protein, enzyme modulator, extracellular matrix protein, hydrolase, nucleic acid binding, phosphatase, receptor, signaling molecule, and transcription factor (FIG. 15B). This result implied that protein class change of cytoskeletal protein and oxidoreductase is independent of CDE-mediated Myd88 signaling. Pathway included Apoptosis signaling pathway, B cell activation, CCKR signaling map, Inflammation mediated by chemokine and cytokine signaling pathway, Interleukin signaling pathway, T cell activation, Toll receptor signaling pathway, and Wnt signaling pathway (FIG. 15B). This result indicated that Myd88 signaling did not utilize Alzheimer diseases-presenilin pathway, Angiogenesis, Cadherin signaling pathway, Cytoskeletal regulation by Rho GTPase, EGF receptor signaling pathway, Huntington disease, Nicotinic acetylcholine receptor signaling pathway, PDGF signaling pathway, Ras Pathway, and VEGF signaling pathway.

### Cat Dander Extract Require Myd88 to Induce Allergic Airway Inflammation

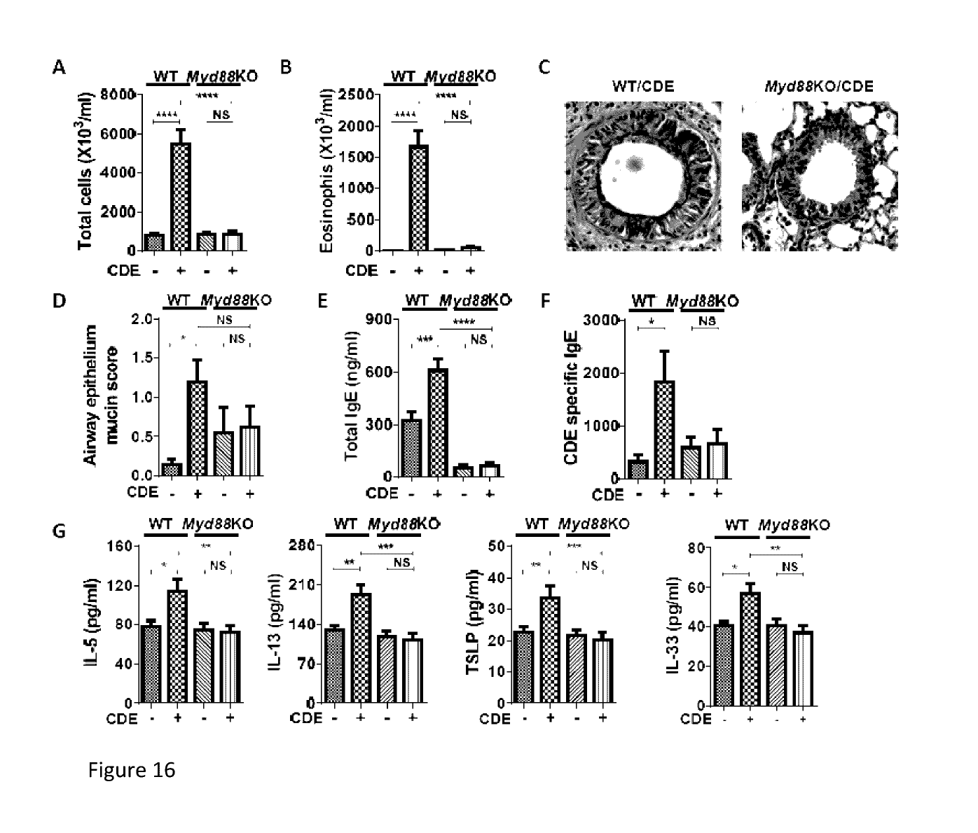

Building on our observation that CDE-induced innate neutrophil recruitment was MyD88 dependent, taken together with earlier reports that innate neutrophil recruitment facilitates allergic inflammation response (Example 1, 28), we hypothesized that MyD88 should modulate CDE-induced allergic inflammation. To test this hypothesis, we utilized repeated challenge model (FIG. 19B). As expected, repeated-challenge with CDE in WT mice induced allergic inflammation characterized by an increase in total cells and eosinophils in BALF (FIG. 16A, B) and stimulation of mucin in airway epithelial cells (FIG. 16C, D). Repeated challenge with CDE in WT mice also increased the levels of total IgE and cat dander specific IgE in serum, and the BALF levels of IL-5, IL-13, IL-33, and TSLP. Consistent with our hypothesis, disruption of MyD88 abolished these measures of allergic inflammation (FIG. 16A-G). These observations indicate that Myd88 is essential for induction of CDE-induced allergic inflammation.

### Identification of Myd88-Dependent CDE-Induced Allergic Immune Response Genes in the Lungs

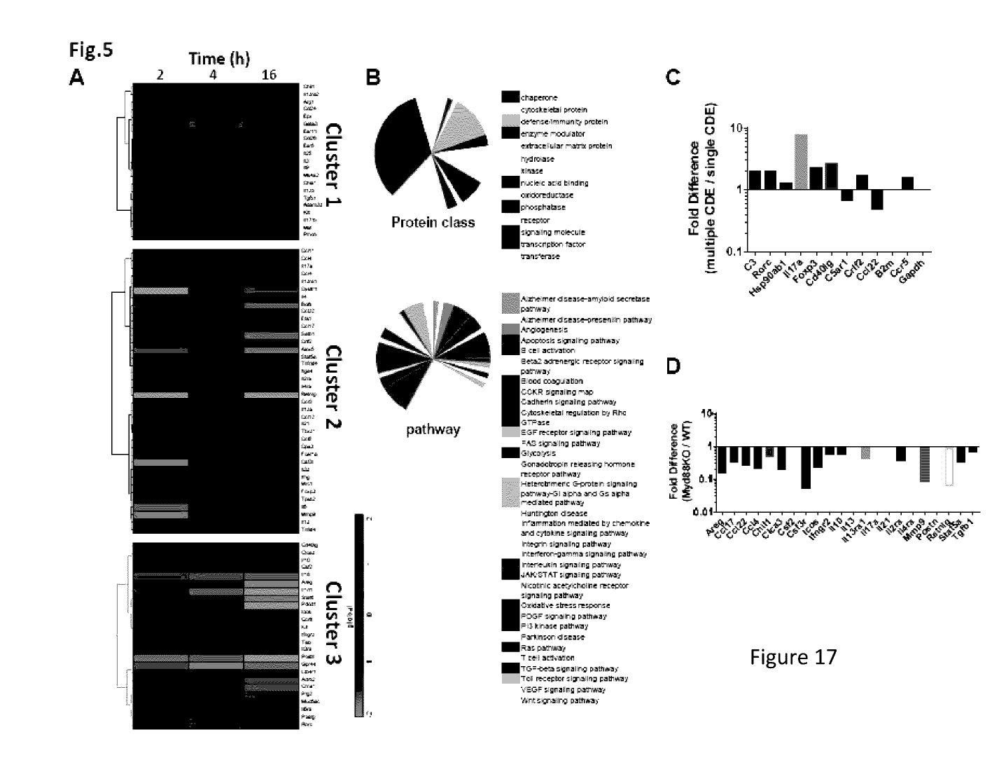

In multiple challenge model, the change of 89 allergic-inflammation associated genes were investigated after 2, 4, and 16 hours-post last challenge. Compared to PBS challenge in WT mice, multiple CDE challenges significantly increased lung mRNA expression. Hierarchical clustering analysis from 2, 4, and 16 hours rendered a dendrogram including three major clusters of transcripts (FIG. 17A). Cluster 1 contained 21 transcripts that were mostly upregulated (FIG. 17A). Cluster 2 contained 31 transcripts that demonstrated a biphasic pattern of an immediate decrease followed by a later increase.

There was no difference in expression of Cxcl1 and Cxcl2 between single challenge and multiple challenges (data not shown). These results indicating that multiple challenge model using CDE is enough to induce Th2 polarization in WT mice.

Next, we performed a PCR array of 155 innate and allergic-inflammation associated genes in the lungs after 2 hours-post last challenge in multiple model to compare the gene expression between single challenge model and multiple challenge model. Panther analysis with revealed that there were 14 protein class and 32 signaling pathway involved with these mRNA changes in multiple challenge model (FIG. 17B). Protein classes included chaperone, cytoskeletal protein, defense/immunity protein, enzyme modulator, extracellular matrix protein, hydrolase, kinase, nucleic acid binding, oxidoreductase, phosphatase, receptor, signaling molecule, transcription factor, and transferase (FIG. 17B). There were the protein classes of chaperone and kinase in multiple challenge model, but not single challenge model. By contrast, there was cell adhesion molecule in single challenge model, but not multiple challenge model. Pathway included Alzheimer diseases-amyloid secretase pathway, Alzheimer diseases-presenilin pathway, Angiogenesis, Apoptosis signaling pathway, B cell activation, Beta2 adrenergic receptor signaling pathway, Blood coagulation, CCKR signaling map, Cadherin signaling pathway, Cytoskeletal regulation by Rho GTPase, EGF receptor signaling pathway, FAS signaling pathway, Glycolysis, Gonadotropin releasing hormone receptor pathway, Heterotrimeric G-protein signaling pathway-Gi alpha and Gs alpha mediated pathway, Huntington disease, Inflammation mediated by chemokine and cytokine signaling pathway, Integrin signaling pathway, Interferon-gamma signaling pathway, Interleukin signaling pathway, JAK/STAT signaling pathway, Nicotinic acetylcholine receptor signaling pathway, Oxidative stress response, PDGF signaling pathway, PI3 kinase pathway, Parkinson disease, Ras pathway, T cell activation, TGF-beta signaling pathway, Toll receptor signaling pathway, VEGF signaling pathway, and Wnt signaling pathway (FIG. 17B). There were Alzheimer diseases-amyloid secretase pathway, Beta2 adrenergic receptor signaling pathway, Blood coagulation, FAS signaling pathway, Glycolysis, Gonadotropin releasing hormone receptor pathway, Heterotrimeric G-protein signaling pathway-Gi alpha and Gs alpha mediated pathway, Integrin signaling pathway, Interferon-gamma signaling pathway, JAK/STAT signaling pathway, Oxidative stress response, PI3 kinase pathway, Parkinson disease, and TGF-beta signaling pathway in multiple challenge model, but not in single challenge model.

Next, we examined differences mRNA expression between single challenge with CDE and multiple challenges with CDE after 2 hours-post last challenge. Compared to single challenge with CDE, multiple challenges with CDE significantly increased lung mRNA expression of C3, Rorc, Hsp90ab1, Il17a, Foxp3, Cd40lg, Crlf2, and Ccr5 in 2 hours after challenge (FIG. 17C). By contrast, lung mRNA expression of C5ar1, Ccl22, B2m, and Gapdh were decreased in multiple challenges model compared to single challenge model (FIG. 17C).

Next, we elucidated the differences in gene expression in allergic immune responses between WT mice and Myd88KO mice in multiple model. We selected 4 hours-post last challenge because 4 hours-post last challenge showed largest difference with WT mice in multiple challenge model. Among the mRNA which was significantly changed by multiple CDE challenge in WT mice, disruption of MyD88 reduced the lung mRNA expression of Areg, Ccl17, Ccl22, Ccl4, Chil1, Clca3, Csf2, Csf3r, Icos, Ifngr2, Il10, Il13ra1, Il17a, Il21, Il2ra, Il4ra, Mmp9, Retn1g, Stat5a, and Tgfb1 (FIG. 16D), and increased lung mRNA expression of Il13 and Postn (FIG. 16D) 4 hours-post challenge.

### Rye Grass Pollen Extract Requires Myd88 to Innately Recruit Neutrophils and Induce Allergic Airway Inflammation

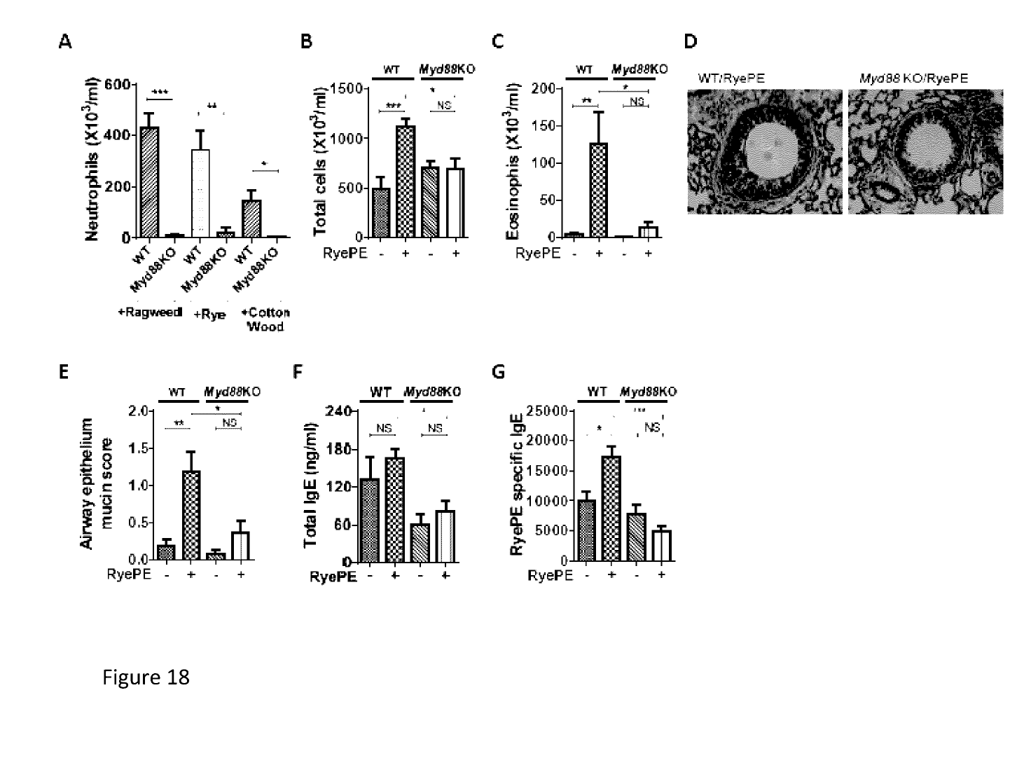

Next we examined the broader role of Myd88 in allergen-induced innate neutrophil recruitment. Pollen allergenic extracts belonging to diverse families—grasses (ragweed and rye), wood (cotton wood) also induced Myd88-dependent innate neutrophil recruitment to the airway (FIG. 18A). Next, we tried to confirm the role of Myd88 in allergic inflammation by repeated challenge model with rye grass pollen extract (RyePE) (FIG. 19B). As expected, repeated-challenge with RyePE in WT mice induced allergic inflammation characterized by an increase in total cells and eosinophils in BALF (FIG. 18B, C) and stimulation of mucin in airway epithelial cells (FIG. 18D, E) and increased the levels of total IgE and RYE-specific IgE in serum (FIG. 18B-E). Consistent with our hypothesis, disruption of MyD88 abolished these measures of allergic inflammation (FIG. 18B-E) Likewise, repeated challenge with CDE in WT mice increased the levels of total IgE and RYE-specific IgE in serum. Disruption of MyD88 abrogated these increases (FIG. 18F, G).

### Discussion

Myd88 is essential adaptor molecule for TLRs signaling to activate innate immune responses such stimulating neutrophil recruitment and secretion of proinflammatory cytokines (31, 32, 40). We have previously reported that RWPE requires TLR4 and MD2 to stimulate an innate neutrophil recruitment that in turn stimulates allergic sensitization and allergic airway inflammation (Example 1). In the present study, we showed that CDE requires Myd88 to stimulate innate recruitment of neutrophil to the lungs and stimulation of allergic sensitization and allergic airway inflammation.

Since gene variants of Myd88 is associated with the incident of atopic dermatitis (41), Myd88 is considered to be associated with the pathogenesis of allergic disease. Because gene expression of TLR2 in the lung was increased by CDE challenge and innate neutrophil recruitment that facilitate allergic inflammation and sensitization by allergen challenge is dependent of TLR2, TLR2/Myd88 might contribute to allergic inflammation by its increase of gene expression with time and repeated exposure. Likewise, Myd88 contributes to airway remodeling since stimulation of TLR2/Myd88 signaling with HDM activates airway smooth muscle cells (42). TLR2/MyD88 signaling modify the property of mast cell (43). TLR4/Myd88 pathway contributes to the pathogenesis of steroid-resistant airway hyperresponsiveness in mice model (44). The activation of TLR4/Myd88 signaling by lambda-carrageenan suppressed allergic immune response in mice (45). Like our study, Myd88 regulates TLR4-dependent TSLP release in allergic conjunctivitis (29). Intranasal OVA challenge-induced allergic inflammation and Th2 cytokine responses are MyD88-dependent through DC activation (46). Taken together, Myd88 is closely involved in the pathogenesis of allergy. To support this, our result of PCR array demonstrated that disruption of MyD88 reduced the allergy-associated lung mRNA expression after repeated CDE challenge.

We have shown that both total IgE and cat dander specific IgE production is lower in Myd88 KO mice compared to WT mice, indicating that allergic sensitization is regulated by Myd88. Since our previous report showed that TLR4-mediated neutrophil recruitment to the airway facilitates antigen-specific IgE production in serum (Example 1), it is likely that Myd88 also regulates IgE production. Furthermore, since neutralization of CXC chemokines decreases the level of IgE in plasma in a mouse model of allergic asthma (47), Myd88-mediated CXC chemokines secretion might directly regulate IgE production. After sensitization by repeated intranasal instillations of OVA, the higher level of serum OVA specific IgE are seen in WT mouse than in MyD88 deficient mouse (46). Our results are consistent with recent study showing that Myd88 signaling in B cell is crucial to produce IgE/IgG1 in exposure to pollen (48). However, the role of neutrophils in class switch of B cells needs to be investigated in the future.

In the present study, the results of PCR array for innate immune response revealed that CDE initiate broad innate inflammatory response through Myd88. The MyD88 adaptor molecule as responsible for the majority of cytokine amplification observed following influenza virus challenge (49). Likewise, our study showed that there was less mRNA expression in Myd88KO mice compared to WT mice generally. It is likely that Myd88 amplify generally cytokine secretion. The difference of gene expressions between single challenge and multiple challenges might give us a clue to understanding how chronic exposure of allergens induces Th2 skewing. In our study, the lung mRNA expression of C3, Rorc, Hsp90ab1, Il17a, Foxp3, Cd40lg, Crlf2, and Ccr5 were higher in multiple challenges than single challenge. Among these gene, only IL17a might be able to initiate allergic inflammation (50), then Th2 polarization might occur characterized with the increase of the expression of Cd40lg, Ccr5, Foxp3, and Crlf2 (TSLP receptor). To support this, the lung mRNA expression of Il17a in Myd88 mice multiple-challenged with CDE was lower than WT mice multiple-challenged with CDE.

Fel d 1 is the most common cat dander protein causing of cat allergy (51). Fel d 1T-cell peptides improve clinical airway symptoms of subjects with cat allergy (52). However, these treatments were not satisfactory for the patients with cat allergy. Mannose receptor (53), or TLR2 and TLR4 (37) might be the new target for the subjects with allergy sensitized with Fel d 1, however, the mechanism of these receptors need to be elucidated furthermore. We suggest that inhibiting recruitment of activated neutrophils by administration of Myd88 inhibitor may be a novel strategy for preventing allergic sensitization and pollen-induced allergic disorders. In addition, inhibition of Myd88 signaling might be effective against neutrophil-dominant forms of asthma, such as severe asthma and sudden-onset asthma (54, 55).

Among outdoor allergens, the NHANES studies identified rye glass as the most common allergen associated with allergic sensitization and allergic diseases (1, 3, 56). NHANES studies showed that the most common allergen in positive reaction of skin test were rye grass (13.2%) in any age groups (56), 44.2% of subjects with hay fever or allergy were sensitized to rye grass (1), 19.5% of subjects they investigated showed positive IgE test to rye grass (3). Rye grass is closely associated with thunderstorm asthma which can cause acute asthma attack (57), by releasing allergen-bearing cytoplasm (58). Ninety-two % of the patients with acute asthma attack had high level of ryegrass-specific IgE antibody (59). Rye grass pollination period comprises only 17% of days of the year (15 October-30 November) in New South Wales, however, this periods includes 53% of the peak asthma count days identified (60).

In summary, we demonstrated that CDE and pollens utilize a novel mechanism that Myd88-dependent neutrophil recruitment facilitates allergic sensitization and allergic airway inflammation. Our insight might be a novel target in the treatment or prevention of allergic sensitization and allergic inflammation.

### REFERENCES FOR EXAMPLE 3

1. Salo et al., J Allergy Clin Immunol 2011; 127:1226-35 e7.
2. Sporik et al., Thorax 1999; 54:675-80.
3. Salo et al., J Allergy Clin Immunol 2014.
4. Roost et al., J Allergy Clin Immunol 1999; 104:941-7.
5. Freidhoff et al., J Allergy Clin Immunol 1984; 73:490-9.
6. Plaschke et al., Allergy 1996; 51:461-72.
7. Linneberg et al., Allergy 2003; 58:21-6.
8. Chew et al., J Allergy Clin Immunol 2008; 121:240-5.
9. Ritz et al., Allergy 2002; 57:357-61.
10. Chen et al., J Allergy Clin Immunol 2007; 119:1148-55.
11. Ronmark et al., J Allergy Clin Immunol 2009; 124:357-63, 63 e1-15.
12. Perzanowski et al., Am J Respir Crit Care Med 2002; 166:696-702.
13. Lau et al., Allergy 2005; 60:766-73.
14. Sarpong et al., Ann Allergy Asthma Immunol 1998; 80:303-8.
15. Sarpong et al., Ann Allergy Asthma Immunol 1997; 79:455-9.
16. Noertjojo et al., J Allergy Clin Immunol 1999; 103:60-5.
17. Chinn et al., Am J Respir Crit Care Med 2007; 176:20-6.
18. Zeidler et al., J Allergy Clin Immunol 2006; 118:1075-81.
19. Wood et al., Am J Respir Crit Care Med 1998; 158:115-20.
20. Corren et al., J Allergy Clin Immunol 2011; 127:398-405.
21. Boldogh et al., J Clin Invest 2005; 115:2169-79.
22. Lommatzsch et al., J Allergy Clin Immunol 2006; 118:91-7.
23. Hoskins et al., Am J Respir Crit Care Med 2013; 187:34-41.
24. Metzger et al., Chest 1986; 89:477-83.
25. Hosoki et al., Am J Respir Cell Mol Biol 2016; 54:81-90.
26. Hosoki et al., J Allergy Clin Immunol 2015.
27. Lammermann et al., Nature 2013; 498:371-5.
28. Oyoshi et al., Immunity 2012; 37:747-58.
29. Li et al., J Allergy Clin Immunol 2011; 128:1318-25 e2.
30. Shalaby et al., J Immunol 2013; 191:922-33.
31. Adachi et al., Immunity 1998; 9:143-50.
32. Kawai et al., Immunity 1999; 11:115-22.
33. Bortolatto et al., Clin Exp Allergy 2008; 38:1668-79.
34. Eisenbarth et al., J Immunol 2004; 172:4527-34.
35. Florsheim et al., J Immunol 2015; 194:4621-30.
36. Brandt et al., J Immunol 2013; 191:3519-25.
37. Herre et al., J Immunol 2013; 191:1529-35.
38. Wild et al., J Immunol 2000; 164:2701-10.
39. Sur et al., J Immunol 1996; 157:4173-80.
40. Nance et al., J Leukoc Biol 2008; 83:1207-17.
41. An et al., Int J Mol Med 2011; 27:795-801.
42. Chiou et al., J Cell Physiol 2009; 220:311-8.
43. Selander et al., J Immunol 2009; 182:4208-16.
44. Yang et al., J Immunol 2009; 182:5107-15.
45. Tsuji et al., Clin Exp Allergy 2003; 33:249-58.
46. Piggott et al., J Clin Invest 2005; 115:459-67.
47. McKinley et al., Cytokine 2005; 32:178-85.
48. Matsushita et al., J Immunol 2014; 193:5791-800.
49. Teijaro et al., Proc Natl Acad Sci USA 2014; 111:3799-804.
50. Song et al., J Immunol 2008; 181:6117-24.
51. Lowenstein et al., Allergy 1985; 40:430-41.
52. Alexander et al., Allergy 2005; 60:1269-74.
53. Emara et al., J Biol Chem 2011; 286:13033-40.
54. Jatakanon et al., Am J Respir Crit Care Med 1999; 160:1532-9.
55. Sur et al., Am Rev Respir Dis 1993; 148:713-9.
56. Gergen et al., J Allergy Clin Immunol 1992; 90:579-88.
57. Suphioglu, Int Arch Allergy Immunol 1998; 116:253-60.
58. Grote et al., J Allergy Clin Immunol 2001; 108:109-15.
59. Pollart et al., J Allergy Clin Immunol 1988; 82:224-30.
60. Hayden et al., N S W Public Health Bull 2011; 22:154-8.

### Example 4

### Abstract

We sought to identify cells and cytokines in bronchoalveolar lavage (BAL) fluids that distinguish asthma from healthy control subjects and those that distinguish controlled asthma from uncontrolled asthma. Following informed consent, 36 human subjects were recruited for this study. These included 11 healthy control subjects, 15 subjects with controlled asthma with FEV₁≧80% predicted and 10 subjects with uncontrolled asthma with FEV₁<80% predicted. BAL fluid was obtained from all subjects. The numbers of different cell types and the levels of 48 cytokines were measured in these fluids. Compared to healthy control subjects, patients with asthma had significantly more percentages of eosinophils and neutrophils, IL-1RA, IL-1α, IL-1β, IL-2Rα, IL-5, IL-6, IL-7, IL-8, G-CSF, GROα (CXCL1), MIP-1β (CCL4), MIG (CXCL9), RANTES (CCL5) and TRAIL in their BAL fluids. The only inflammatory markers that distinguished controlled asthma from uncontrolled asthma were neutrophil percentage and IL-8 levels, and both were inversely correlated with FEV₁. We examined whether grouping asthma subjects on the basis of BAL eosinophil % or neutrophil % could identify specific cytokine profiles. The only differences between neutrophil-normal asthma (neutrophil ≦2.4%) and neutrophil-high asthma (neutrophils %>2.4%) were a higher BAL fluid IL-8 levels, and a lower FEV₁ in the latter group. By contrast, compared to eosinophil-normal asthma (eosinophils ≦0.3%), eosinophil-high asthma (eosinophils >0.3%) had higher levels of IL-5, IL-13, IL-16, and PDGF-bb, but same neutrophil percentage, IL-8, and FEV₁. Our results identify neutrophils and IL-8 are the only inflammatory components in BAL fluids that distinguish controlled asthma from uncontrolled asthma, and both correlate inversely with FEV₁.

### Introduction

Asthma is a complex chronic inflammatory disorder of the airways with a high prevalence rate of approximately 300 million people worldwide (1). Severe asthma represents approximately 5 to 10% of all subjects with asthma (2), but accounts for 40% of the total cost for asthma care (2) and 30-50% of asthma morbidity (3). The National Heart, Lung, and Blood Institute's Severe Asthma Research Program (SARP) demonstrated that reduced FEV₁ (forced expiratory volume in 1 second), history of pneumonia, and fewer positive skin tests for environmental allergens were critical independent risk factors for severe asthma (4). However, the SARP study also reported that the well-established biomarkers of asthma, such as blood eosinophils, serum IgE, and exhaled nitric oxide levels, do not differentiate asthma severity or correlate with FEV₁ or asthma severity (4). The RET/ATS guidelines have been changed for defining asthma severity to controlled and uncontrolled asthma. It is important to identify specific cytokines that distinguish uncontrolled asthma from controlled asthma in the new guideline to develop novel therapeutic targets for severe asthma.

Increasing evidence suggests that inflammatory cells in the airways can distinguish severe asthma from mild asthma (5-12). Because sputum samples are collected non-invasively, several studies have evaluated sputum samples, and reported higher percentages of neutrophils in the sputum in severe compared to mild asthma (7, 8). However, because sputum neutrophil numbers do not correlate with the cell numbers in bronchoalveolar lavage (BAL) fluids from the same subjects (13), it is important to validate the observations of neutrophilia in the sputum by sampling other compartments of the airways. A study of tracheal aspirates from patients intubated for acute severe asthma reported a higher percentage of neutrophils compared to a control group of patients undergoing nonpulmonary surgical procedures (9). In another study, patients intubated for status asthmaticus exhibited a higher mean percentage of neutrophils in their BAL fluid compared to that from patients with stable mild asthma (10). We have reported that unlike classic slow-onset progressive fatal asthma, peribronchial lung tissues in sudden-onset fatal asthma had considerably more neutrophils than eosinophils (6). Thus an increasing body of literature supports the idea that there is an abundance of neutrophils in severe asthma.

Many cytokines and chemokines could theoretically be associated with “neutrophil-rich” and “eosinophil-rich” endotypes of asthma (14). However, most studies have utilized a candidate cytokine approach to quantify specific cytokines in asthma (9-12). One such candidate cytokine study evaluated sputum concentrations of IL-8, and reported higher levels in severe vs. mild asthma (7). Another study evaluated IL-8 in tracheal aspirates, and reported higher levels in patients intubated for acute severe asthma compared to a control group of patients undergoing surgical procedures unrelated to the lung (9). Likewise, the concentration of IL-8 in BAL fluid from patients intubated for status asthmaticus was elevated compared to mild asthma (10). To our knowledge, only two study evaluated an array of over 20 cytokines and chemokines in BAL fluid to identify cytokines that distinguish severe asthma from mild or moderate asthma (15, 16). One of these studies reported identically level of IL-8 in moderate and severe asthma in children compared to adult controls (15), whereas the other reported no difference in BAL fluid levels of IL-8 between mild asthma and severe asthma (16). To address this difference in the observations reported in candidate-cytokine studies (9-12) vs. panel-cytokine study (16), we examined a panel of 48 cytokines and chemokines in BAL fluids from healthy control subjects and subjects with controlled and uncontrolled asthma.

### Materials and Methods

### Subjects

Subjects were recruited in the Department of Asthma, Allergy and Lung Biology, King's College London School of Medicine, U.K. The study was approved by the Ethics Committee of King's College Hospital, and each participant provided written informed consent. Subjects with asthma were included on the basis of history and a demonstrated reversible airflow limitation (20% variability in forced expiratory volume in one second [FEV₁] or peak expiratory flow rate), increased airway responsiveness to methacholine (concentration producing a decrease of 20% from base line in FEV₁ [PC₂₀], <8 mg per millilitre), or both. None had ever smoked, and there was no history of other respiratory disease. Atopy was defined as the presence of one or more positive skin prick tests to a range of common aeroallergens. The normal controls had no history of allergic disease, had normal FEV₁, and a PC₂₀ of more than 32 mg per millilitre. Of the controls, 5 of 11 were atopic. The subjects' characteristics are shown in Table 1. These included 11 healthy control subjects (FEV₁=102%, 89-110), 15 subjects with controlled asthma (Mean FEV₁ 98%, 81-113) and 10 with uncontrolled asthma (Mean FEV₁ 64%, 48-74, <80%). For the purpose of this study, we defined asthma severity based on FEV₁ while on treatment, according to international ERS/ATS guidelines (17).

TABLE 1
Patient characteristics.

| | | Controlled | Uncontrolled |
|---|---|---|---|
| Characteristic | Healthy | Asthma | Asthma |
| n | 11 | 15 | 10 |
| Age (yr) | 24.0 (19-38) | 27.1 (19-41) | 45.8 (29-63)*⁺ |
| Sex (% male) | 45.5 | 40 | 60 |
| Use of ICS (%) | none | 11.7 | 100 |
| Mean dose of ICS | none | 27 | 710 |
| Use of LABA | none | none | 90 |
| (%) | | | |
| Duration of | NA | 4.7 (3-13) | 10.0 (6-20) |
| asthma, (yr) | | | |
| Atopy, % | 54.5 | 66.7 | 70 |
| FEV1, | 102.0 (89-118) | 98.1 (83-113) | 64.1 (48-74)*⁺ |
| % predicted | | | |
| Total IgE (IU/ml) | 49.6 (17-232) | 86.7 (19-623)* | 123.2 (18-721)* |
| Blood eosinophils | 0.6 (0.2-2.6) | 2.1 (0.6-3.2)* | 2.9 (0.4-4.1)* |
| (% leukocytes) | | | |

Results expressed in means and range.
*Statistical significant compared to healthy group
⁺Statistical significant compared to controlled asthma

### Fiberoptic Bronchoscopy and Collection of BAL Fluid

Fiberoptic bronchoscopy was performed, and BAL fluid obtained and processed as previously described (18). Briefly, bronchoscopy was performed by the same operator in both the asthmatics and the controls after they had received 2.5 mg of albuterol by nebulizer, 0.6 mg of atropine, midazolam for sedation, and 2% or 4% of lidocaine for local anaesthesia. BAL was performed by instilling four 60-ml aliquots of warmed, pH-adjusted, normal saline into either the right middle lobe or the lingula. After collection, BAL cells were centrifuged at 300×g for 7 min, washed once, and resuspended in 1.5 mL of PBS; BAL fluid supernatants were distributed into 10 ml each tube and stored at −80° C. for further analysis (up to 3 years). The mean total amount of BAL fluid was 92 ml.

### Cell Counts in BAL Fluid

Cytospin slides of BAL cells were made with a Shandon 2 cytospin device (Shandon Southern Instruments, Runcorn, UK). For cell differentiation, slides were stained with May-Grunwald Giemsa. Cell counts were performed and the absolute numbers and percentages of eosinophils, neutrophils, lymphocytes and monocytes/macrophages were quantified.

### Cytokines and Chemokines in BAL Fluid

Cytokines in BAL fluid were quantified using a Bio-Plex array for 48 cytokines (Bio-Rad, Hercules, Calif.) according to the manufacturer's instructions: Interleukin (IL)-1α, IL-1β, IL-1 receptor antagonist (IL-1RA), IL-2, IL-2Rα, IL-3, IL-4, IL-5, IL-6, IL-7, IL-8, IL-9, IL-10, IL-12 (p40), IL-12 (p70), IL-13, IL-15, IL-16, IL-17, IL-18, fibroblast growth factor (FGF), eotaxin (CCL11), granulocyte colony-stimulating factor (G-CSF), granulocyte-macrophage colony-stimulating factor (GM-CSF), interferon (IFN)-γ, interferon gamma-induced protein (IP)-10/CXCL10, monocyte chemotactic protein (MCP)-1/CCL2, macrophage inflammatory protein (MIP)-1α/CCL3, MIP-1β/CCL4, platelet-derived growth factor (PDGF), regulated-on-activation normal T-cell expressed and secreted (RANTES)/CCL5, tumor necrosis factor (TNF)-α, vascular endothelial growth factor (VEGF), cutaneous T cell attracting chemokine (CTACK)/CCL27, growth regulated oncogene α (GROα)/CXCL1, hepatocyte growth factor (HGF), IFN-α2, leukemia inhibitory factor (LIF), MCP-3/CCL7, macrophage colony-stimulating factor (M-CSF), macrophage migration inhibitory factor (MIF), monokine induced by interferon-gamma (MIG)/CXCL9, nerve growth factor-β (NGF-β), stem cell factor (SCF), stem cell growth factor-β (SCGF-β), stromal cell-derived factor-1α (SDF-1α), TNF-β, and TNF-related-apoptosis-induced-ligand (TRAIL). The lower limits of detection of cytokines that were not detected (ND) or were at borderline limits of detection were: IL-2 (3 pg/ml), IL-4 (6 pg/ml), IL-13 (4 pg/ml), IL-17 (48 pg/ml), FGF (172.4 pg/ml), CCL3 (178 pg/ml), CCL11 (325 pg/ml), GM-CSF (22 pg/ml), and TNF-α (543 pg/ml).

### Statistical Analysis

The results of the study are presented as means±SEM. Group comparisons were analyzed by an unpaired Student's t-test or one-way ANOVA with Tukey's multiple comparisons test. The Holm procedure was used for multiple comparison adjustment. Linear regression analysis was performed to assess the relationship among parameters. For the logistic regression comparing asthmatic and healthy subjects, an elastic net regression was used with leave-half-out validation for model selection and error estimation. For the logistic regression comparing controlled to uncontrolled asthma, a least squares regression predicting FEV₁%, stepwise selection was used with the Bayesian Information Criterion as the model selection criterion. All calculations were performed in R (version 3.0.2). The software package GraphPad Prism 6 (GraphPad Software, San Diego, Calif.) was used for the preparation of graphs. Statistical significance was set at p<0.05.

### Results

Differences in BAL Fluid Cellular and Cytokine Profiles of Subjects with Asthma Vs. Healthy Controls

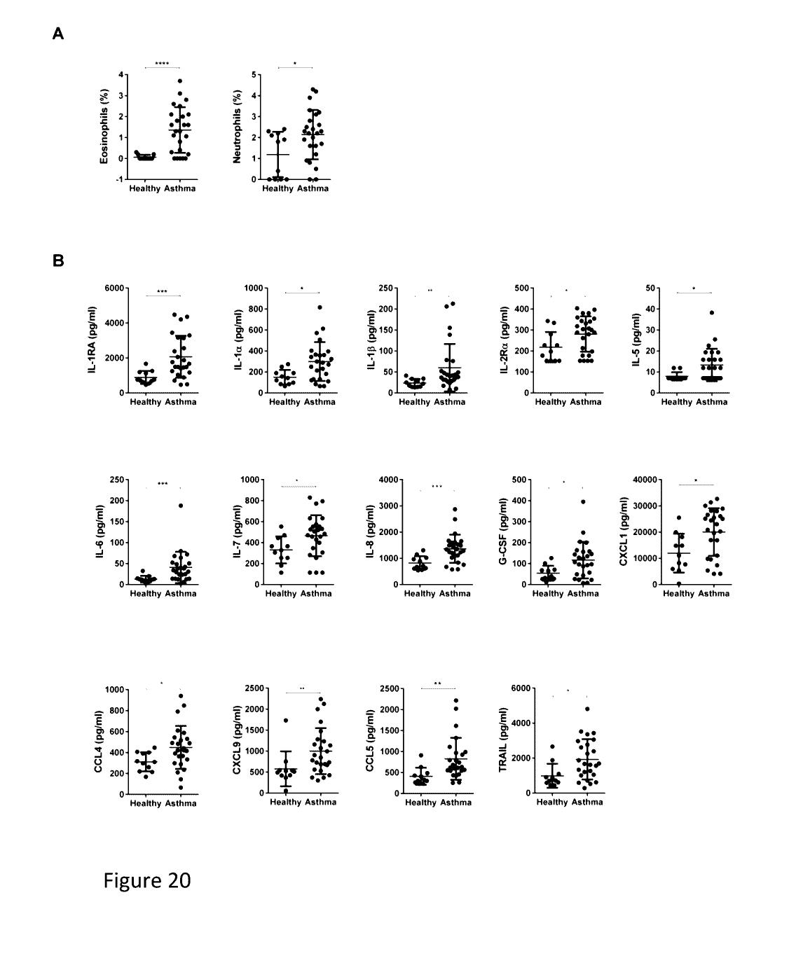

Compared to healthy control subjects (n=11), subjects with asthma (n=25) had higher % eosinophils (p<0.001) and % neutrophils (p<0.05) in their BAL fluids (FIG. 20A). Furthermore, subjects with asthma had 2.3-fold higher IL-1RA (p<0.001), 2.0-fold higher IL-1α (p<0.05), 2.5-fold higher IL-1β (p<0.01), 1.3-fold higher IL-2Rα (p<0.05), 1.7-fold higher IL-5 (p<0.05), 3.2-fold higher IL-6 (p<0.001), 1.4-fold higher IL-7 (p<0.05), 1.7-fold higher IL-8 (p<0.001), 2.2-fold higher G-CSF (p<0.05), 1.7-fold higher CXCL1 (p<0.05), 1.4-fold higher CCL4 (p<0.05), 1.7-fold higher CXCL9 (p<0.01), 2.0-fold higher CCL5 (p<0.01) and 1.9-fold higher TRAIL (p<0.05) concentrations in their BAL fluids (FIG. 20B). By contrast, subjects with asthma and healthy controls had similar mean concentrations of such other cytokines as IL-3, IL-9, IL-10, IL-12 (p40), IL-12 (p70), IL-13, IL-15, IL-16, IL-18, IFN-γ, IFN-α2, CXCL10, CCL2, CCL3, PDGF-bb, VEGF, CCL27, HGF, LIF, CCL7, M-CSF, MIF, NGF-β, SCF, SCGF-β, SDF-1α and TNF-β (Table 2). IL-2, IL-4, IL-17, CCL11, FGF, GM-CSF, and TNF-α were not detected in either group. Thus, 14 out of 48 cytokines were higher in subjects with asthma, compared to healthy control subjects.

TABLE 2
Cellular and cytokine profile in BAL fluid without statistical
difference between subjects with asthma and healthy controls.

| Cellular component | | |
|---|---|---|
| and cytokines | Healthy | Asthmatic |
| IL-2 (pg/ml) | ND | ND |
| IL-3 (pg/ml) | 90.4 ± 103.9 | 138.2 ± 110.2 |
| IL-4 (pg/ml) | ND | ND |
| IL-9 (pg/ml) | 25.0 ± 9.8 | 31.5 ± 10.9 |
| IL-10 (pg/ml) | 12.8 ± 12.5 | 14.3 ± 11.9 |
| IL-12 (p40) (pg/ml) | 693.5 ± 231.0 | 811.8 ± 240.4 |
| IL-12 (p70) (pg/ml) | 237.1 ± 82.0 | 233.1 ± 94.4 |
| IL-13 (pg/ml) | 12.0 ± 5.2 | 14.3 ± 6.3 |
| IL-15 (pg/ml) | 6.1 ± 4.5 | 5.8 ± 3.8 |
| IL-16 (pg/ml) | 1034.1 ± 935.3 | 1317.6 ± 749.7 |
| IL-17 (pg/ml) | ND | ND |
| IL-18 (pg/ml) | 282.0 ± 102.9 | 408.4 ± 262.2 |
| FGF (pg/ml) | ND | ND |
| CCL11 (pg/ml) | ND | ND |
| GM-CSF (pg/ml) | ND | ND |
| IFN-α2 (pg/ml) | 105.3 ± 57.1 | 126.0 ± 47.0 |
| IFN-γ (pg/ml) | 73.6 ± 70.2 | 88.5 ± 89.8 |
| CXCL10 (pg/ml) | 13362.5 ± 19043.1 | 14797.3 ± 10298.6 |
| CCL2 (pg/ml) | 349.5 ± 122.5 | 403.9 ± 132.9 |
| CCL3 (pg/ml) | ND | ND |
| PDGF-bb (pg/ml) | 95.1 ± 94.2 | 192.5 ± 147.9 |
| TNF-α (pg/ml) | ND | ND |
| VEGF (pg/ml) | 3304.9 ± 1427.3 | 3358.5 ± 1534.4 |
| CCL27 (pg/ml) | 365.0 ± 144.6 | 416.0 ± 134.5 |
| HGF (pg/ml) | 554.5 ± 286.6 | 748.4 ± 324.3 |
| LIF (pg/ml) | 150.3 ± 82.9 | 168.3 ± 88.0 |
| CCL7 (pg/ml) | 522.9 ± 301.4 | 588.2 ± 243.5 |
| M-CSF (pg/ml) | 274.7 ± 103.7 | 369.1 ± 157.2 |
| MIF (pg/ml) | 9627.4 ± 8797.7 | 14690.1 ± 8929.0 |
| NGF-β (pg/ml) | 340.8 ± 87.5 | 383.7 ± 89.5 |
| SCF (pg/ml) | 413.5 ± 222.8 | 450.1 ± 203.1 |
| SCGF-β (pg/ml) | 775.1 ± 546.1 | 647.5 ± 498.0 |
| SDF-1α (pg/ml) | 1118.5 ± 477.5 | 1427.2 ± 478.7 |
| TNF-β (pg/ml) | 582.6 ± 187.4 | 630.5 ± 208.7 |

Results expressed in means and range.
ND; Not detected.

BAL Cytokine Profile Analysis Identify Only IL-8 Levels and % Neutrophils as Biomarker that Distinguish Controlled Asthma from Uncontrolled Asthma, and Both Correlate Inversely with FEV₁

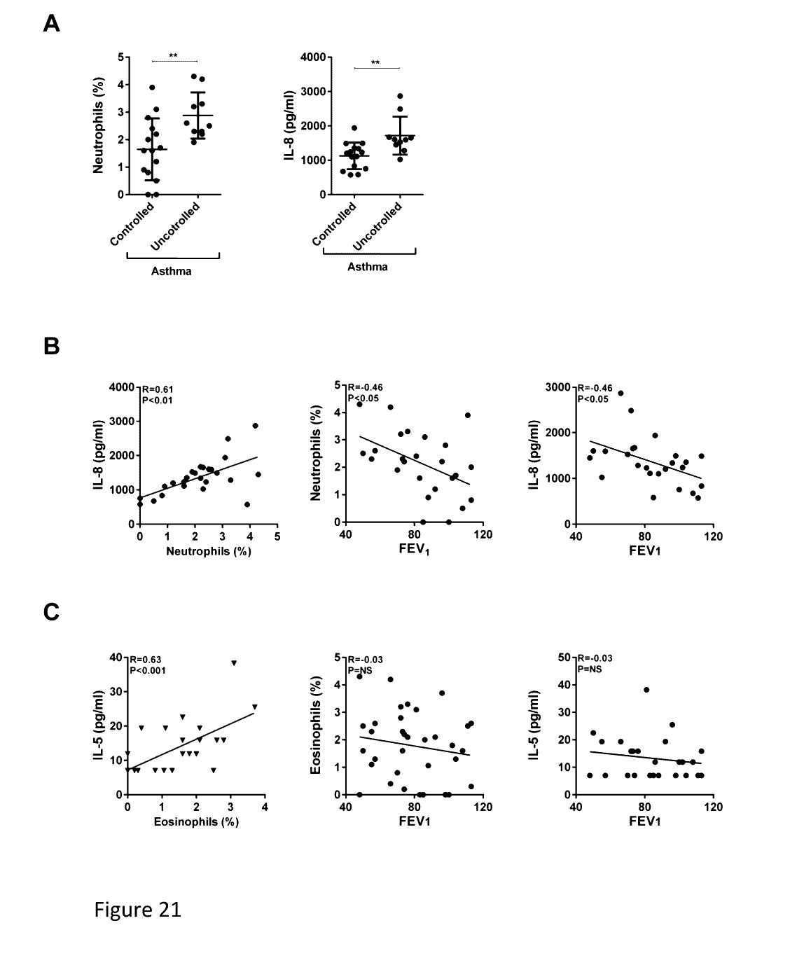

Next, we determined which of these cells and 14 cytokines (FIG. 20) elevated in asthma distinguished controlled from uncontrolled asthma. Unexpectedly, there were only two differences between these two groups. Subjects with uncontrolled asthma had a mean 1.7-fold higher percentage of neutrophils in the BAL fluid compared to those with controlled asthma (controlled asthma=1.6±1.1%, uncontrolled asthma=2.9±0.8%, p<0.01, FIG. 21A). The mean concentration of IL-8 in the BAL fluid from subjects with uncontrolled asthma was 1.5-fold higher than that in subjects with controlled asthma (controlled asthma=1128±386 pg/ml, uncontrolled asthma=1716±551 pg/ml, p<0.01, FIG. 21A). Furthermore, only IL-8 concentrations in all subjects with asthma (controlled and uncontrolled) significantly correlated with the percentages of neutrophils in the BAL fluid (R=0.61, p<0.01, FIG. 21B). In addition, the percentages of neutrophils and the concentrations of IL-8 in the BAL fluid were both inversely correlated with the % predicted FEV₁ (R=−0.46, p<0.05 for both neutrophil % and IL-8 levels, FIG. 21B). Even though BAL eosinophil % in all subjects with asthma correlated with BAL fluid IL-5 levels (FIG. 21C), neither eosinophil % nor IL-5 levels correlated with % predicted FEV₁ (FIG. 21C). Some cytokines elevated in subjects with asthma significantly correlated with the level of IL-8 in BAL fluids: IL1-RA (R=0.59, p<0.01), IL-1α (R=0.40, p<0.05), IL-6 (R=0.68, p<0.001), IL-7 (R=0.47, p<0.05), G-CSF (R=0.74, p<0.0001), CCL4 (R=0.45, p<0.05), CXCL1 (R=0.64, p<0.01), and CXCL9 (R=0.48, p<0.05). However, these cytokines did not correlate with the % neutrophils or % predicted FEV₁ in BAL fluids.

Next we statistically examined whether inhaled corticosteroid (ICS) could have contributed to some of the observations in the present study by separating all subjects with asthma into those that received ICS vs. those that did not. Subjects with asthma that were being treated with ICS had higher % neutrophils (p<0.05), higher IL-8 levels (p<0.05) and lower % predicted FEV₁ (p<0.0001). However, the dose of ICS did not correlate the level of % neutrophils and IL-8 levels in BAL fluids (data not shown).

Eosinophil-High and Neutrophil-High Asthma have Different Cytokine Profiles and FEV₁

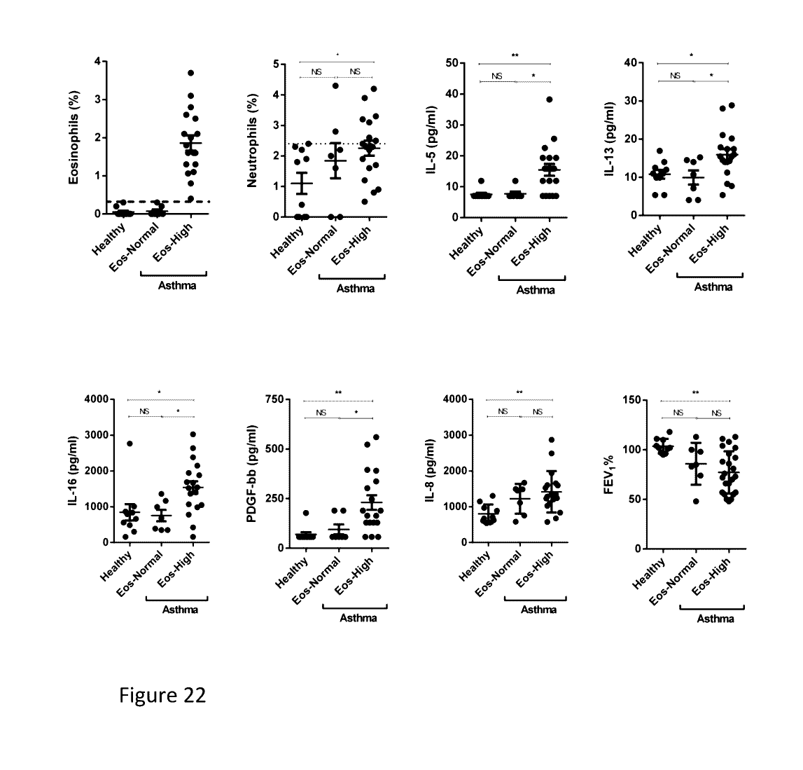

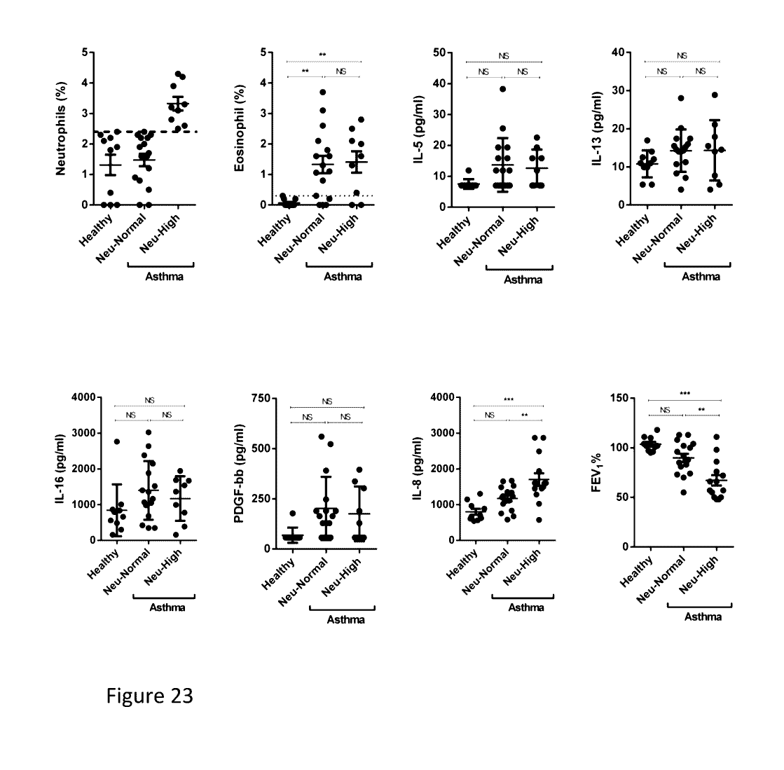

Building on the unexpected observation that % neutrophil but not % eosinophils correlated inversely with % predicted FEV₁ in asthma, we examined whether grouping asthma subjects on the basis of BAL eosinophil % or neutrophil % could identify specific cytokine profiles. In our study, the upper limit of percent of eosinophils and neutrophils in the BAL fluid of healthy subjects was 0.3% and 2.4%, respectively (FIGS. 22 and 23). For the purpose of this study, we separated all subjects with asthma into either eosinophil-high (eosinophils >0.3%, Eos-High) and eosinophil-normal (eosinophils ≦0.3%, Eos-Normal) groups (FIG. 22), or neutrophil-high (neutrophils %>2.4%, Neu-High), and neutrophil-normal (neutrophil ≦2.4%, Neu-Normal) groups (FIG. 23). Compared to Eos-Normal asthma, Eos-High asthma had higher levels of IL-5 (p<0.05), IL-13 (p<0.05), IL-16 (p<0.05), and PDGF-bb (p<0.05), but same % neutrophils, IL-8, other cytokines (data not shown), and FEV₁ (FIG. 22). By contrast, compared to Neu-Normal asthma, Neu-High asthma had higher IL-8 levels (p<0.01) and lower % predicted FEV₁ (p<0.01), but similar levels of eosinophil %, IL-5, IL-13, IL-16, and PDGF-bb (FIG. 23) and other cytokines and chemokines (data not shown). These results also indicate an association of Neu-High asthma with IL-8 and % FEV₁.

### Multiple Regression Analysis Models

The estimated predictive equation for the presence of asthma using logistic regression was: Log it (Present (asthma))=−3.85+0.0033 (IL-8)+2.77 (% eosinophils) (p=0.05 and 0.09, respectively). The accuracy of this model was 84%, with 89% sensitivity and 75% specificity. The predictive equation for FEV₁% predicted in asthma was 103-0.023 (IL-8)+0.040 (IL-1α). The R² for this model was 0.34 (p=0.0037 and 0.06, respectively). Atopy had no significant effect.

### Discussion

Prior studies have mostly measured candidate cytokines, and reported increased levels of IL-8 and neutrophils in the sputum in severe asthma (7). Our study of the BAL fluid provides this specific information by demonstrating that IL-8 is the only cytokine among 48 measured that is significantly elevated in uncontrolled asthma. The higher BAL fluid IL-8 levels in uncontrolled asthma seen in our study could reflect persistent stimulation of IL-8 secretion by chronic stimulation of the nuclear factor-KB signaling pathway following exposure to environmental factor (19), or intrinsic differences in the ability of uncontrolled asthma patients' airway epithelium to produce high amounts of IL-8 (20). In addition to its ability to stimulate neutrophil recruitment, IL-8 may contribute to the pathogenesis of severe asthma by directly facilitating airway remodeling by increasing bronchial smooth muscle cell migration and proliferation (21), inducing airway hyperresponsiveness (AHR) (22), and stimulating epithelial-mesenchymal transition (EMT) (23) in the airways.

In our study, neutrophil-high asthma had lower FEV₁, and the neutrophil percentage in asthma was inversely correlated with FEV₁ and directly correlated with IL-8 levels. The mechanistic contribution of neutrophils to asthma severity is not well understood, and our study was not designed to address this issue. A variety of factors produced by neutrophils could theoretically contribute to the pathogenesis of severe asthma. Depletion of neutrophils in a mouse model of allergic asthma has been reported to reduce AHR and airway remodeling (24). Matrix metalloproteinase 9 (MMP-9) from neutrophils has been shown to be associated with asthma severity (24). Neutrophil elastase can induce AHR (25), and promote the EMT (26). After interacting with allergens, neutrophils release α-defensins (27), which can stimulate IL-8 secretion from human bronchial epithelial cells (28). Neutrophils from subjects with asthma produce higher TGF-β1 (29), a strong inducer of the EMT. Neutrophils are a major source of reactive oxygen species (ROS) generated by gp91phox NADPH oxidase (30), and promote allergic airway inflammation (31).

In our study, 12% of the subjects with controlled asthma and all subjects with uncontrolled asthma used ICS. Because steroids can inhibit apoptosis of neutrophils (32) and suppress eosinophil survival (33), use of ICS could have impacted the results of our study by skewing cell counts to higher neutrophilia in uncontrolled asthma. However, in our study the dose of ICS did not correlated the level of neutrophils in BAL fluids, suggesting that this is most likely not the explanation for higher % neutrophil. As in our study, others have also reported elevated neutrophils in severe asthma, independent of steroids. For example, the European Network for Understanding Mechanisms of Severe Asthma study also reported more neutrophils in the sputum from subjects with severe asthma, independent of corticosteroid use (34). Likewise, use of inhaled corticosteroids did not impact BAL fluid IL-8 levels in a study of the molecular phenotyping of severe asthma (16). Further studies are needed to clarify the effect of ICS on neutrophils and eosinophils in the airways (35).

Consistent with prior studies (11, 12, 36-43), our results also demonstrate that subjects with asthma have higher concentrations of IL-5 and the numbers of eosinophils in BAL fluid compared to control subjects. This is not surprising because eosinophilic inflammation is a significant feature of the pathology of asthma (9-12, 36, 37, 44). In our study, eosinophils and IL-5 did not correlate with percent predicted FEV₁. The lack of association between eosinophils and FEV₁ in asthma is surprising because eosinophils have been shown to contribute to AHR in murine, guinea pig and mammal studies (45-48). However, several human studies have shown that eosinophils do not correlate with AHR or airflow obstruction (49-51).

It is somewhat surprising some Th2 cytokines and chemokines, especially IL-4, IL-13, and CCL11 were not elevated in the present study, even though prior reports indicated the increase of these cytokines and chemokines (38-43, 52-58). Two studies performed in the 1990s reported elevated IL-4 levels in concentrated BAL fluids in asthma (40, 41). Since concentrating BAL fluid may induce a processing artifact, more recent studies have been performed on unconcentrated BAL fluids (15, 59). Like our study that was also performed on unconcentrated BAL fluids using multiplex beads, these studies reported that IL-4 and IL-13 were undetectable in unconcentrated BAL fluids in asthma (15, 59). Like our study, a previous study reported that there is no elevation of TNFα or GM-CSF in BAL fluids from the subjects of asthma (15). Prior studies have reported an increase in CCL11 levels in BAL fluids in subjects with asthma after allergen challenge (56), and CCL11 positive cells or CCL11 mRNA expression in bronchial biopsy specimens in asthma (57, 58). However, other studies that were similar to ours, and sampled the BAL compartment in asthma without allergen challenge, also failed to detect CCL11 (60), or detected CCL11 at a level that would be too low (7-41 pg/ml) to be detectable by our kit (lower limit of detection 325 pg/ml) (15, 59).

We unexpectedly did not detect IL-17 in our study. A recent study reported that IL-17 is present in BAL fluids from the subjects with asthma at mean levels of about 60 pg/ml (25-150 pg/ml) (61). Since the lower limit of detection level of IL-17 in our study 48 pg/ml, this could account for failure to detect IL-17 in our study.

Recent studies have suggested that asthma is a heterogeneous disease complex that should be classified into distinct endotypes based on their cytokine profiles (15, 16, 62-65). In the present study we also show there are quantitative differences in cytokine pattern between neutrophil-high asthma and eosinophil-high asthma. However, our data suggest that uncontrolled and controlled asthma have a fairly uniform cytokine profile and may have a common pathogenesis instead of being a collection of fundamentally distinct diseases. Our observations question the importance cytokine-based endotypes classification of asthma in predicting asthma severity.

The specific association of only IL-8 in 48 cytokines quantified in BAL fluids with neutrophil-high and uncontrolled asthma in the present study provides specificity to earlier candidate-cytokine studies reporting elevated IL-8 and neutrophils in severe asthma (7-10). Together, these studies indicate that the mechanistic role of IL-8 and recruited neutrophils should be carefully evaluated in uncontrolled asthma. CXCR2 is one of the receptors for IL-8 (66). A recent study demonstrated that CXCR2 inhibitor reduced sputum neutrophilia and asthma exacerbations, and improved Asthma Control Questionnaire (ACQ) score in patients with severe asthma (67). If the results of our study are confirmed in mechanistic and large-scale BAL fluid studies, inhibition of neutrophil recruitment by CXCR2 inhibitors and others agents should be explored as alternate therapeutic strategies in uncontrolled asthma with elevated neutrophils.

### REFERENCES FOR EXAMPLE 3

1. Braman, Chest. 2006; 130(1 Suppl):4S-12S.
2. Godard et al., Eur Respir J. 2002; 19(1):61-7.
3. Anagnostou et al., Prim Care Respir J. 2012; 21(1):71-7.
4. Moore et al., J Allergy Clin Immunol. 2007; 119(2):405-13.
5. Wenzel et al., Am J Respir Crit Care Med. 1999; 160(3):1001-8.
6. Sur et al., Am Rev Respir Dis. 1993; 148(3):713-9.
7. Jatakanon et al., Am J Respir Crit Care Med. 1999; 160(5 Pt 1):1532-9.
8. Moore et al., J Allergy Clin Immunol. 2013.
9. Ordonez et al., Am J Respir Crit Care Med. 2000; 161(4 Pt 1):1185-90.
10. Lamblin et al., Am J Respir Crit Care Med. 1998; 157(2):394-402.
11. Sur et al., J Allergy Clin Immunol. 1995; 96(5 Pt 1):661-8.
12. Sur et al., J Allergy Clin Immunol. 1996; 97(6):1272-8.
13. Arron et al., Eur Respir J. 2013. Epub 2013/11/02
14. Wenzel, Nat Med. 2012; 18(5):716-25.
15. Fitzpatrick et al., J Allergy Clin Immunol. 2010; 125(4):851-7 e18.
16. Brasier et al., J Allergy Clin Immunol. 2008; 121(1):30-7 e6.
17. Chung et al., Eur Respir J. 2014; 43(2):343-73.
18. Ying et al., J Immunol. 2008; 181(4):2790-8.
19. Gagliardo et al., Am J Respir Crit Care Med 2003; 168(10):1190-8.
20. Gras et al., J Allergy Clin Immunol. 2012; 129(5):1259-66 e1.
21. Kuo et al., Toxicol Sci. 2011; 121(1):177-90.
22. Xiu et al., Clin Exp Allergy. 1995; 25(1):51-9.
23. Fernando et al., Cancer Res. 2011; 71(15):5296-306.
24. Park et al., J Immunol. 2006; 176(4):2538-45.
25. Suzuki et al., Am J Respir Crit Care Med. 1996; 153(4 Pt 1):1405-11.
26. Grosse-Steffen et al., Clin Dev Immunol. 2012; 2012:720768.
27. Vega et al., J Investig Allergol Clin Immunol. 2011; 21(1):38-43.
28. Sakamoto et al., Am J Physiol Lung Cell Mol Physiol. 2005; 288(3):L508-13.
29. Chu et al., J Allergy Clin Immunol. 2000; 106(6):1115-23.
30. Hampton et al., Blood. 1998; 92(9):3007-17.
31. Sevin et al., Am J Respir Cell Mol Biol. 2013; 49(3):396-402.
32. Cox, J Immunol. 1995; 154(9):4719-25.
33. Cox et al., Am J Respir Cell Mol Biol. 1991; 4(6):525-31.
34. The ENFUMOSA cross-sectional European multicentre study of the clinical phenotype of chronic severe asthma. European Network for Understanding Mechanisms of Severe Asthma. Eur Respir J. 2003; 22(3):470-7.
35. Green et al., Thorax. 2002; 57(10):875-9.
36. Virchow et al., Am J Respir Crit Care Med. 1995; 151(4):960-8.
37. Tillie-Leblond et al., Am J Respir Crit Care Med. 2000; 162(2 Pt 1):586-92.
38. Ying et al., J Immunol. 1997; 158(7):3539-44.
39. Humbert et al., Am J Respir Crit Care Med. 1996; 154(5):1497-504.
40. Walker et al., Am J Respir Crit Care Med. 1994; 150(4):1038-48.
41. Walker et al., Am Rev Respir Dis. 1992; 146(1):109-15.
42. Leung et al., J Exp Med. 1995; 181(1):33-40.
43. Robinson et al., N Engl J Med. 1992; 326(5):298-304.
44. Wardlaw et al., Am Rev Respir Dis. 1988; 137(1):62-9.
45. Lee et al., Science. 2004; 305(5691):1773-6.
46. Justice et al., Am J Physiol Lung Cell Mol Physiol. 2003; 284(1):L169-78.
47. Verbout et al., Am J Physiol Lung Cell Mol Physiol. 2009; 297(2):L228-37.
48. Gundel et al., J Clin Invest. 1991; 87(4):1470-3.
49. Brightling et al., Thorax. 2003; 58(6):528-32.
50. Flood-Page et al., Am J Respir Crit Care Med. 2003; 167(2):199-204.
51. Leckie et al., Lancet. 2000; 356(9248):2144-8.
52. Huang et al., J Immunol. 1995; 155(5):2688-94.
53. Kotsimbos et al., Proc Assoc Am Physicians. 1996; 108(5):368-73.
54. Kroegel et al., Eur Respir J. 1996; 9(5):899-904.
55. Moller et al., Am J Respir Cell Mol Biol. 1996; 14(5):439-43.
56. Lilly et al., Am J Respir Crit Care Med. 2001; 163(7):1669-75.
57. Lamkhioued et al., J Immunol. 1997; 159(9):4593-601.
58. Ying et al., Eur J Immunol. 1997; 27(12):3507-16.
59. Bossley et al., J Allergy Clin Immunol. 2012; 129(4):974-82 e13.
60. Woodman et al., Chest. 2006; 130(2):371-8.
61. Irvin et al., J Allergy Clin Immunol. 2014; 134(5):1175-86 e7.
62. Anderson, Lancet. 2008; 372(9643):1107-19.
63. Moore et al., Am J Respir Crit Care Med. 2010; 181 (4):315-23.
64. Haldar et al., Am J Respir Crit Care Med. 2008; 178(3):218-24.
65. Woodruff et al., Am J Respir Crit Care Med. 2009; 180(5):388-95.
66. Emadi et al., Blood. 2005; 105(2):464-73.
67. Nair et al., Clin Exp Allergy. 2012; 42(7):1097-103.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
administering to a subject a composition comprising a CXCR2 inhibitor, wherein the subject has an allergic airway inflammation or is at risk of an allergic airway inflammation, and wherein at least one sign of the allergic airway inflammation or the risk of having the allergic airway inflammation is reduced.

2. The method of claim 1 wherein the allergic airway inflammation comprises an allergic asthma.

3. The method of claim 1 wherein the allergic airway inflammation comprises a clinical sign selected from allergic conjunctivitis, allergic rhinitis, allergic cutaneous inflammation, and a combination thereof.

4. The method of claim 3 wherein the allergic cutaneous inflammation comprises atopic dermatitis.

5. The method of claim 2 wherein the subject has uncontrolled asthma.

6. The method of claim 2 wherein the subject has controlled asthma.

7. The method of claim 1 wherein the subject does not have asthma.

8. The method of claim 1 wherein the allergic airway inflammation comprises an allergic rhinitis.

9. The method of claim 1 wherein the method further comprises determining (i) the level of IL-8 in bronchoalveolar (BAL) fluid of the subject, (ii) the neutrophil percentage in BAL fluid of the subject, or the combination thereof.

10. The method of claim 1 wherein the CXCR2 inhibitor is a CXCR2 antagonist.

11. The method of claim 10 wherein the CXCR2 antagonist is an anti-CXCR2 antibody.

12. The method of claim 1 wherein the composition is administered by inhalation.

13. The method of claim 1 wherein the composition is administered orally.

14. The method of claim 1 wherein the allergic airway inflammation comprises an inflammatory response to an allergen, wherein the allergen is an aeroallergen.

15. The method of claim 14 wherein the aeroallergen comprises a pollen, an arachnid antigen, a fungal cell, an animal antigen, an insect antigens, or a combination thereof.

16. The method of claim 15 wherein the aeroallergen comprises a pollen, an animal antigen, or a combination thereof.

17. The method of claim 12 wherein the animal antigen comprises an animal dander.

18. The method of claim 12 wherein the aeroallergen comprises a pollen.

19. A method comprising:
administering to a subject a composition comprising an MD2 inhibitor, wherein the subject has an allergic airway inflammation or is at risk of an allergic airway inflammation, and wherein at least one sign of the allergic airway inflammation or the risk of having the allergic airway inflammation is reduced.

20. A method comprising:
administering to a subject a composition comprising an MyD88 inhibitor, wherein the subject has an allergic airway inflammation or is at risk of an allergic airway inflammation, and wherein at least one sign of the allergic airway inflammation or the risk of having the allergic airway inflammation is reduced.
